SEQUENCE LISTING (101 sequences)

SEQ ID NO: 1 - DNA, 195 bp, M. tuberculosis
atggcgcaagagcagaccaagcgtggcggtggcggcggcgatgatgacgacatcgccggcagcaccgccgcgggccaggagcgtcgcgaaaagctgaccgaggagaccgacgatctgctcgacgaaatcgacgacgtcctcgaggagaacgccgaggacttcgtccgcgcatacgtccaaaagggcggacagtga

SEQ ID NO: 2 - protein, 64 aa, M. tuberculosis
MAQEQTKRGGGGGDDDDIAGSTAAGQERREKLTEETDDLLDEIDDVLEENAEDFVRAYVQKGGQ

SEQ ID NO: 3 - DNA, 29 bp, Artificial Sequence
  Synthetic Oligonucleotide
gctctagacatggcgcaagagcagaccaa

SEQ ID NO: 4 - DNA, 28 bp, Artificial Sequence
  Synthetic Oligonucleotide
ggggtacctcactgtccgcccttttgga

SEQ ID NO: 5 - DNA, 34 bp, Artificial Sequence
  Synthetic Oligonucleotide
ggaattccatatgggtgagtcagagcgttctgag

SEQ ID NO: 6 - DNA, 34 bp, Artificial Sequence
  Synthetic Oligonucleotide
ctcgagtgcggccgcgccctacaggtactggccg

SEQ ID NO: 7 - DNA, 31 bp, Artificial Sequence
  Synthetic Oligonucleotide
gctctagagatgggtgagtcagagcgttctg

SEQ ID NO: 8 - DNA, 28 bp, Artificial Sequence
  Synthetic Oligonucleotide
ggggtacctcgctggtgaccgcctgagc

SEQ ID NO: 9 - DNA, 28 bp, Artificial Sequence
  Synthetic Oligonucleotide
ccgagctcatggcgcaagagcagaccaa

SEQ ID NO: 10 - DNA, 28 bp, Artificial Sequence
  Synthetic Oligonucleotide
ggggtacctcactgtccgcccttttgga

SEQ ID NO: 11 - DNA, 34 bp, Artificial Sequence
  Synthetic Oligonucleotide
gggaattccatatgagaggatcgcatcaccatca

SEQ ID NO: 12 - DNA, 20 bp, Artificial Sequence
  Synthetic Oligonucleotide
atcaacaggagtccaagctc

SEQ ID NO: 13 - DNA, 31 bp, Artificial Sequence
  Synthetic Oligonucleotide
ggaattccatatgattgcgttgctcgcaccc

SEQ ID NO: 14 - DNA, 37 bp, Artificial Sequence
  Synthetic Oligonucleotide
aaaactgcagttataggtttgccagctcgtccaggtc

SEQ ID NO: 15 - DNA, 58 bp, Artificial Sequence
  Synthetic Oligonucleotide
ggaattccatatggattacaaggatgacgacgataagatcattgcgttgctcgcaccc

SEQ ID NO: 16 - DNA, 61 bp, Artificial Sequence
  Synthetic Oligonucleotide
cccaagcttactgcagtcagtggtggtggtggtggtgtaggtttgccagctcgtccaggtc

SEQ ID NO: 17 - DNA, 31 bp, Artificial Sequence
  Synthetic Oligonucleotide
ccccaattgttatggcgcaagagcagaccaa

SEQ ID NO: 18 - DNA, 25 bp, Artificial Sequence
  Synthetic Oligonucleotide
ccatcgatctgtccgcccttttgga

SEQ ID NO: 19 - DNA, 32 bp, Artificial Sequence
  Synthetic Oligonucleotide
ccccaattgttatgattgcgttgctcgcaccc

SEQ ID NO: 20 - DNA, 32 bp, Artificial Sequence
  Synthetic Oligonucleotide
ccatcgattaggtttgccagctcgtccaggtc

SEQ ID NO: 21 - DNA, 28 bp, Artificial Sequence
  Synthetic Oligonucleotide
aaaactgcagagcgcaagagcagaccaa

SEQ ID NO: 22 - DNA, 29 bp, Artificial Sequence
  Synthetic Oligonucleotide
cccaagctttcactgtccgcccttttgga

SEQ ID NO: 23 - DNA, 29 bp, Artificial Sequence
  Synthetic Oligonucleotide
aaaactgcagaattgcgttgctcgcaccc

SEQ ID NO: 24 - DNA, 36 bp, Artificial Sequence
  Synthetic Oligonucleotide
cccaagcttttataggtttgccagctcgtccaggtc

SEQ ID NO: 25 - DNA, 26 bp, Artificial Sequence
  Synthetic Oligonucleotide
ccccaattggcgcaagagcagaccaa

SEQ ID NO: 26 - DNA, 28 bp, Artificial Sequence
  Synthetic Oligonucleotide
ccatcgattcactgtccgcccttttgga

SEQ ID NO: 27 - DNA, 27 bp, Artificial Sequence
  Synthetic Oligonucleotide
ccccaattgattgcgttgctcgcaccc

SEQ ID NO: 28 - DNA, 35 bp, Artificial Sequence
  Synthetic Oligonucleotide
ccatccatttataggtttgccagctcgtccaggtc

SEQ ID NO: 29 - protein, 33 aa, Mycobacterium tuberculosis
REIVEHSYLVAQAHQIVEQAHKVALEASSSGRA

SEQ ID NO: 30 - protein, 20 aa, Mycobacterium tuberculosis
KLGAAMGADVTVLSQSLKKM

SEQ ID NO: 31 - protein, 25 aa, Mycobacterium tuberculosis
RAHPELAEQVAKELDVHVTSQDARE

SEQ ID NO: 32 - protein, 25 aa, Mycobacterium tuberculosis
KLTKEQAEYLGVDVEGPYKPDHYRY

SEQ ID NO: 33 - protein, 20 aa, Mycobacterium tuberculosis
RTKALVENMSDQDIWNLKRG

SEQ ID NO: 34 - protein, 24 aa, Mycobacterium tuberculosis
RADNSPHVVAVGFTFDPKTHIARV

SEQ ID NO: 35 - protein, 20 aa, Mycobacterium tuberculosis
KAAEAWSGGYFAKSVVPVRD

SEQ ID NO: 36 - protein, 19 aa, Mycobacterium tuberculosis
RAKVEKDPENPTVVLTVRG

SEQ ID NO: 37 - protein, 26 aa, Mycobacterium tuberculosis
RGVEKGYVLGPQAEDNVWDLTPEERR

SEQ ID NO: 38 - protein, 19 aa, Mycobacterium tuberculosis
RNVVVLGPGTIPKTPSGKL

SEQ ID NO: 39 - protein, 20 aa, Mycobacterium tuberculosis
RRGFNIESLAVGATECKDRS

SEQ ID NO: 40 - protein, 25 aa, Mycobacterium tuberculosis
RVGSGPFPTELFDEHGEYLSKTGRE

SEQ ID NO: 41 - protein, 28 aa, Mycobacterium tuberculosis
RALSAAGNGDAVVQPSGAGIHEVDVKIT

SEQ ID NO: 42 - protein, 21 aa, Mycobacterium tuberculosis
RTVSLPVGADEDDIKATYDKG

SEQ ID NO: 43 - protein, 19 aa, Mycobacterium tuberculosis
RAELPGVDPDKDVDIMVRD

SEQ ID NO: 44 - protein, 19 aa, Mycobacterium tuberculosis
KGILTVSVAVSEGKPTEKH

SEQ ID NO: 45 - protein, 11 aa, Mycobacterium tuberculosis
RTEQKDFDGRS

SEQ ID NO: 46 - protein, 12 aa, Mycobacterium tuberculosis
DPDKDVDIMVRD

SEQ ID NO: 47 - protein, 16 aa, Mycobacterium tuberculosis
RLTALEKLAEDPPAKA

SEQ ID NO: 48 - protein, 14 aa, Mycobacterium tuberculosis
RALIKDLSFSLPRN

SEQ ID NO: 49 - protein, 19 aa, Mycobacterium tuberculosis
RVGAGEKLGFAQNDIELRG

SEQ ID NO: 50 - protein, 25 aa, Mycobacterium tuberculosis
KAAPVGLFVETFGTETEDPVKIEKA

SEQ ID NO: 51 - protein, 25 aa, Mycobacterium tuberculosis
KSGAIKFDEVDITTLQGAELANYRR

SEQ ID NO: 52 - protein, 13 aa, Mycobacterium tuberculosis
KFVAAFDVDAKKV

SEQ ID NO: 53 - protein, 17 aa, Mycobacterium tuberculosis
RLFKALVDAASGYAGRQ

SEQ ID NO: 54 - protein, 18 aa, Mycobacterium tuberculosis
RVIGATLSAEQAKWGQKA

SEQ ID NO: 55 - protein, 18 aa, Mycobacterium tuberculosis
RWAEITDLSGAKIAGFKL

SEQ ID NO: 56 - protein, 14 aa, Mycobacterium tuberculosis
RTLVTGKSSSASRA

SEQ ID NO: 57 - protein, 16 aa, Mycobacterium tuberculosis
RGFSEDPKALEPAVRK

SEQ ID NO: 58 - protein, 25 aa, Mycobacterium tuberculosis
RNSGDKPEVVYFEPDKLSTGSANRC

SEQ ID NO: 59 - protein, 19 aa, Mycobacterium tuberculosis
RITAPDEPKYDADFAAVRQ

SEQ ID NO: 60 - protein, 16 aa, Mycobacterium tuberculosis
KLTEEAADKLTEVIKN

SEQ ID NO: 61 - protein, 22 aa, Mycobacterium tuberculosis
RIITAATVVLPFKIDDHSAWRL

SEQ ID NO: 62 - protein, 18 aa, Mycobacterium tuberculosis
RMIGAGELVSAAGKALRD

SEQ ID NO: 63 - protein, 13 aa, Mycobacterium tuberculosis
KFQKELAAMGFKF

SEQ ID NO: 64 - protein, 18 aa, Mycobacterium tuberculosis
KATTFFDDPDVLAKVSRG

SEQ ID NO: 65 - protein, 19 aa, Mycobacterium tuberculosis
RDAIIDDASTFFKQQEERG

SEQ ID NO: 66 - protein, 21 aa, Mycobacterium tuberculosis
RKMAADFPTISLVNSVNPVRI

SEQ ID NO: 67 - protein, 12 aa, Mycobacterium tuberculosis
KVTETLLKGAKE

SEQ ID NO: 68 - protein, 18 aa, Mycobacterium tuberculosis
RNFLVENADVADEIEKKI

SEQ ID NO: 69 - protein, 18 aa, Mycobacterium tuberculosis
RNSIDLYHKQAADTGARI

SEQ ID NO: 70 - protein, 11 aa, Mycobacterium tuberculosis
RMTTAVDKARV

SEQ ID NO: 71 - protein, 11 aa, Mycobacterium tuberculosis
RGWSLIKSVRL

SEQ ID NO: 72 - protein, 16 aa, Mycobacterium tuberculosis
RSVEQAKALHEMLERR

SEQ ID NO: 73 - protein, 22 aa, Mycobacterium tuberculosis
KDSYSFDIDAAGLKAAYHAHRE

SEQ ID NO: 74 - protein, 15 aa, Mycobacterium tuberculosis
RTQAVAAAEVLGKRQ

SEQ ID NO: 75 - protein, 16 aa, Mycobacterium tuberculosis
RKATTDEPFAALAFKI

SEQ ID NO: 76 - protein, 28 aa, Mycobacterium tuberculosis
RLTPGDKAPAFTLPDADGNNVSLADYRG

SEQ ID NO: 77 - protein, 11 aa, Mycobacterium tuberculosis
KATGHVAKLRR

SEQ ID NO: 78 - protein, 15 aa, Mycobacterium tuberculosis
RYAAATSQTKGLIFG

SEQ ID NO: 79 - protein, 11 aa, Mycobacterium tuberculosis
KEQADKILAKL

SEQ ID NO: 80 - protein, 13 aa, Mycobacterium tuberculosis
RLEVDAKEIIGRY

SEQ ID NO: 81 - protein, 12 aa, Mycobacterium tuberculosis
KVAKVTWDQVRE

SEQ ID NO: 82 - protein, 12 aa, Mycobacterium tuberculosis
KLVKIETDIPRG

SEQ ID NO: 83 - protein, 11 aa, Mycobacterium tuberculosis
RAEVLGKSLRH

SEQ ID NO: 84 - protein, 13 aa, Mycobacterium tuberculosis
RASMPKFLSTARG

SEQ ID NO: 85 - protein, 14 aa, Mycobacterium tuberculosis
RAEKYTQVETLVYM

SEQ ID NO: 86 - protein, 34 aa, Mycobacterium tuberculosis
KAGMIGITQALAPGLAAKGITINAVAPGFIETQM

SEQ ID NO: 87 - protein, 27 aa, Mycobacterium tuberculosis
RGATTALVYLSPDAKPAATGLESTMRF

SEQ ID NO: 88 - protein, 14 aa, Mycobacterium tuberculosis
GADGKLGAFVEVKN

SEQ ID NO: 89 - protein, 9 aa, Mycobacterium tuberculosis
RDVLAVVSK

SEQ ID NO: 90 - protein, 22 aa, Mycobacterium tuberculosis
LGSAGEGGDGGVGGNGGGGAKG

SEQ ID NO: 91 - protein, 15 aa, Mycobacterium tuberculosis
NAEDFVRAYVQKGGQ

SEQ ID NO: 92 - protein, 15 aa, C. jeikeium
NAEEFVRSYVQKGGQ

SEQ ID NO: 93 - protein, 15 aa, Homo sapiens
QKESTLHLVLRLRGG

SEQ ID NO: 94 - protein, 14 aa, Mycobacterium tuberculosis
LTALEKLAEDPPAK

SEQ ID NO: 95 - protein, 15 aa, Streptomyces coelicoloris
NAEDFVRSFVQKGGE

SEQ ID NO: 96 - protein, 15 aa, Nocardioides
NAEDFVKSFIQKGGE

SEQ ID NO: 97 - protein, 15 aa, Corynebacterium glutamicum
NAEEFVRSYVQKGGE

SEQ ID NO: 98 - protein, 15 aa, Acidothermus cellulolyticus
NAEEFVRSYIQKGGE

SEQ ID NO: 99 - protein, 15 aa, Frankia alni
NAEEFVKGYVQKGGE

SEQ ID NO: 100 - DNA, 9 bp, Artificial Sequence
  Synthetic Oligonucleotide
agaggatcg

SEQ ID NO: 101 - DNA, 15 bp, Artificial Sequence
  Synthetic Oligonucleotide
ggatccgcatgcgag

(19) United States
(12) Patent Application Publication
(10) Pub. No.: US 2010/0055715 A1
(43) Pub. Date: Mar. 4, 2010

(54) Nucleic and amino acid sequences of prokaryotic ubiquitin-like protein and methods of use thereof

(76) Inventors: Michael James Pearce, Brooklyn, NY (US); Katerina Heran Darwin, New York, NY (US)

Correspondence Address: KLAUBER & JACKSON, 411 HACKENSACK AVENUE, HACKENSACK, NJ, 07601, US

(21) Appl. No.: 12/455,500

(22) Filed: Jun. 2, 2009

Related U.S. Application Data
(60) Provisional application No. 61/130,759, filed on Jun. 2, 2008.

(51) Int. Cl. G01N 33/573; G01N 33/53; C12N 15/63; C07K 14/35; C07K 16/00; C07H 21/04

(52) U.S. Cl. 435/7.4; 435/7.1; 435/320.1; 530/323; 530/387.9; 530/388.1; 530/389.1; 536/23.7

(57) ABSTRACT

The present invention relates to prokaryotic ubiquitin-like protein (Pup) nucleic acid sequences and Pup amino acid sequences encoded therefrom. Also encompassed are antibodies that are immunologically specific for Pup. Methods directed to isolation and identification of pupylated substrates that utilize the conjugated Pup as an affinity tag are also included. Pupylated substrates that are identified using the method and reagents of the present invention provide tools useful for the identification of additional components of prokaryotic proteasomal machinery. Modulators of Pup activity and methods for identifying such modulators are also encompassed herein.

### CROSS REFERENCE TO RELATED APPLICATIONS

This application claims priority under 35 USC §119(e) from U.S. Provisional Application Ser. No. 61/130,759, filed Jun. 2, 2008, which application is herein specifically incorporated by reference in its entirety.

The research leading to the present invention was funded in part by NIH Grant No. 1R56AI065437-01A2 and 1R01HL092774-01 and NIH Training Grant No. 5T32 AI07189-25. The United States government has certain rights in the invention.

### FIELD OF THE INVENTION

The present invention pertains to the fields of molecular biology, regulated protein degradation, and diagnostic and therapeutic medicine. More specifically, the invention relates to prokaryotic ubiquitin-like protein (Pup) nucleic acid sequences and amino acid sequences encoded thereby, and antibodies immunologically specific for Pup. Methods of making and using Pup nucleic and amino acid sequences and Pup antibodies are also encompassed by the present invention.

### BACKGROUND OF THE INVENTION

Several publications and patent documents are referenced in this application in order to more fully describe the state of the art to which this invention pertains. The disclosure of each of these publications and documents is incorporated by reference herein.

Mycobacterium tuberculosis (Mtb) is one of the world's deadliest pathogens, claiming about 1.5 million lives annually (Dye et al., Lancet Infect Dis 8, 233 (April, 2008). The occurrence of approximately 9 million new cases of Mtb a year and the increased emergence of antibiotic resistant strains necessitates the development of new anti-mycobacterial drugs. The Mtb proteasome and proposed cofactors, Mycobacterium proteasomal ATPase (Mpa) and proteasome accessory factor A (PafA), are essential for the pathogenicity of Mtb (Darwin et al., Science 302, 1963 (2003); Darwin et al, Mol Microbiol 55, 561 (2005); Gandotra et al, Nat Med 13, 1515 (December, 2007)), qualifying components of the Mtb proteasome system as potential drug targets.

Similar to the eukaryotic 20S proteasome, the Mtb proteasome is a multi-subunit barrel-shaped protease composed of two rings of catalytic β-subunits sandwiched by rings of α-subunits (Benaroudj et al, Mol Cell 11, 69 (January, 2003); Groll et al., Nature 386, 463 (Apr. 3, 1997); Hu et al., Mol. Microbiol. 59, 1417 (2006); Lin et al., Mol. Microbiol. 59, 1405 (2006); Unno et al., Structure 10, 609 (May, 2002)). The eukaryotic 26S proteasome is composed of a 20S core particle and one or two 19S regulatory caps, which include ATPases that recognize, unfold, and translocate substrates into the core for degradation [reviewed in Baumeister et al., Cell 92, 367 (1998)]. In Mtb, Mpa shares homology with regulatory cap ATPases that translocate proteins into the core. The present inventors previously identified substrates of the Mtb proteasome (Pearce et al., EMBO J. 25, 5423 (2006)), however, the mechanism(s) whereby these substrates were targeted for degradation was not elucidated. Proteins delivered to the eukaryotic proteasome are usually conjugated with ubiquitin, which covalently attaches to substrate lysines (Lys) as well as onto ubiquitin itself [reviewed in Hershko et al, Annu Rev Biochem 67, 425 (1998)]. Ubiquitin-like genes have not been identified in the Mtb genome, suggesting that substrate targeting to the Mtb proteasome occurs via an ubiquitin-independent mechanism.

### SUMMARY OF INVENTION

The protein modifier ubiquitin is a signal for proteasome-mediated degradation in eukaryotes. Prior to the instant discovery, proteasome-bearing prokaryotes have been thought to degrade proteins via an ubiquitin-independent pathway. As described herein, the present inventors have identified a prokaryotic ubiquitin-like protein, Pup (Rv2111c), which specifically conjugates to proteasome substrates in the pathogen Mycobacterium tuberculosis (Mtb). Pupylation occurs on lysines and requires proteasome accessory factor A (PafA). In a pafA mutant, pupylated proteins are absent and substrates accumulate, thereby linking pupylation with degradation. Although analogous to ubiquitylation, pupylation appears to proceed by a different chemistry. The identification of this novel protein modifier in prokaryotes directly impacts the study of bacterial proteolysis, extends the field of ubiquitin-like protein research, and provides new insights regarding the origin of ubiquitin.

In accordance with the results presented herein, the present invention is directed to an isolated nucleic acid sequence encoding a polypeptide comprising SEQ ID NO: 2 or having sequence and/or structural homology to SEQ ID NO: 2, or a functional fragment thereof, wherein said polypeptide exhibits an activity of Pup, for example, is conjugated to proteasome substrates and conjugation thereof is associated with substrate degradation. In one embodiment, a polypeptide having sequence and/or structural homology to SEQ ID NO: 2 or a functional fragment thereof is a Pup homolog or ortholog that exhibits a Pup activity. Also included are expression vectors comprising an isolated nucleic acid sequence which encodes an amino acid sequence of the invention (e.g., SEQ ID NO: 2), wherein expression of the nucleic acid sequence is controlled by regulatory sequences in the expression vector. Cells comprising such expression vectors are also encompassed.

In another aspect of the invention, an isolated amino acid sequence comprising a polypeptide of SEQ ID NO: 2, or having sequence and/or structural homology to either SEQ ID NO: 2, or a functional fragment thereof, wherein said polypeptide is capable of exhibiting a Pup activity, is presented. As described herein, Pup activities include covalent conjugation to proteasome substrates, which is associated with proteasome-mediated substrate degradation. Also included are expression vectors encoding an amino acid sequence of the invention (e.g., SEQ ID NO: 2), wherein expression of the amino acid sequence is controlled by regulatory sequences in the expression vector, and cells comprising such expression vectors.

In another aspect of the invention, an isolated nucleic acid sequence comprising SEQ ID NO: 1 is presented, wherein the nucleic acid sequence encodes Pup or a functional fragment thereof capable of exhibiting an activity attributable to Pup as described herein. Also described is an expression vector comprising a nucleic acid sequence of SEQ ID NO: 1, wherein the nucleic acid sequence encodes Pup or functional fragment thereof capable of exhibiting a Pup activity, and SEQ ID NO: 1 is operably linked to a regulatory sequence. Moreover, a cell comprising such an expression vector is also within the scope of the invention.

The present invention also encompasses an antibody immunologically specific for an amino acid sequence comprising SEQ ID NO: 2. Such antibodies can be polyclonal or monoclonal antibodies and functional fragments thereof.

The present invention also includes a kit comprising an isolated nucleic acid sequence comprising SEQ ID NO: 1, wherein the nucleic acid sequence encodes Pup or functional fragment thereof; an isolated nucleic acid sequence encoding an amino acid sequence comprising SEQ ID NO: 2 or a functional fragment thereof; an isolated amino acid sequence comprising SEQ ID NO: 2, wherein the amino acid sequence is Pup or functional fragment thereof; a Pup activity compatible buffer; at least one antibody immunologically specific for Pup; and instructional materials.

Also described is a composition comprising at least one Pup or functional fragment thereof, a Pup encoding nucleic acid sequence, at least one antibody immunologically specific for Pup, and/or a Pup modulatory agent identified using the methods of the invention and a pharmaceutically acceptable buffer.

In another aspect of the present invention, a method for identifying a proteasome substrate in a bacterial cell is presented, the method comprising:

a) isolating polypeptides covalently conjugated to Pup from a bacterial cell; and
b) characterizing the polypeptides covalently conjugated to Pup to identify each of the polypeptides conjugated to Pup, wherein identifying each of the polypeptides conjugated to Pup identifies a proteasome substrate in the bacterial cell. In a particular embodiment the bacteria is a Mycobacterium species. As is understood in the art, Mycobacterium is a genus Actinobacteria, which include M. tuberculosis and M. leprae.

In accordance with this embodiment, polypeptides covalently conjugated to Pup may be isolated by affinity purification. In a particular embodiment, polypeptides covalently conjugated to Pup are affinity purified by virtue of binding the Pup moiety conjugated thereto. In a particular embodiment, polypeptides covalently conjugated to Pup are isolated by affinity purification using antibodies immunologically specific for Pup.

In certain embodiments, the bacterial cell expresses a tagged form of Pup comprising Pup and a tag (e.g., an epitope tag) and polypeptides covalently conjugated to the tagged form of Pup are isolated by affinity purification of the tag. In accordance with the invention, polypeptides covalently conjugated to Pup are characterized by sequencing at least part of each of the polypeptides conjugated to Pup.

Pup substrates so identified may be used as targets for the development of therapeutics. In addition, the identification of Pup substrates will provide insight into why their accumulation in a proteasome-defective strain attenuates Mtb in vivo.

In yet another aspect of the invention, a method for identifying modulators of Pup activity in a bacterial cell is presented, the method comprising:

(a) providing at least two bacterial cells, wherein each bacterial cell expresses Pup;
(b) incubating at least one bacterial cell expressing Pup in the presence of an agent and at least one cell expressing Pup in the absence of the agent;
c) isolating and characterizing polypeptides covalently conjugated to Pup from the at least one bacterial cell expressing Pup in the presence of the agent and the at least one cell expressing Pup in the absence of the agent; and
d) comparing the polypeptides covalently conjugated to Pup from the at least one bacterial cell expressing Pup in the presence of the agent to the polypeptides covalently conjugated to Pup from the at least one cell expressing Pup in the absence of the agent, wherein a change in the population of polypeptides covalently conjugated to Pup in the presence and absence of the agent identifies an agent that is a modulator of Pup activity in the bacterial cell.

In a particular embodiment, the change in the population of polypeptides covalently conjugated to Pup is a change in an amount of a particular polypeptide covalently conjugated to Pup or a change in types of polypeptides conjugated to Pup. With respect to types of polypeptides conjugated to Pup, the term types refers to polypeptides having different primary sequences or the spectrum of polypeptides categorized based on amino acid sequence.

In a particular embodiment, the bacteria is a Mycobacterium species, including, but not limited to, M. tuberculosis and M. leprae.

In a particular embodiment, the at least two bacterial cells express a tagged form of Pup comprising Pup and a tag and polypeptides covalently conjugated to the tagged form of Pup are isolated by affinity purification of the tag.

In yet another particular embodiment, polypeptides covalently conjugated to Pup are characterized by sequencing at least part of each of the polypeptides conjugated to Pup.

The present invention also encompasses a method for screening to identify Pup “mimetics” or “traps”. The invention further encompasses Pup “mimetics” or “traps” so identified. The term Pup “mimetics” or “traps” includes competitive inhibitors or dominant negative forms of Pup that would be capable of partially or fully inhibiting proteasome function, which is associated with pathogenicity of Mtb. Accordingly, the present invention further encompasses methods of using Pup traps identified using the method of the present invention as therapeutics for the treatment of patients (mammals and, more particularly, humans) with bacterial infections.

In yet another aspect of the invention, a method for identifying an enzyme that covalently attaches Pup to a proteasome substrate in a bacterial cell is presented, the method comprising:

(a) providing a population of bacterial cells;
(b) introducing an expression library of nucleic acid sequences isolated from a Pup expressing bacterial cell into the population of bacterial cells to generate a population of bacterial cells expressing exogenous nucleic acids;
(c) characterizing polypeptides covalently conjugated to Pup in the population of bacterial cells expressing exogenous nucleic acids to identify an exogenous nucleic acid that increases covalent conjugation of Pup polypeptides when expressed.

Enzymes involved in the covalent attachment of Pup to substrates may be referred to herein as activation and/or conjugation enzymes of Pup.

In an embodiment of the method, the population of bacterial cells comprises proteasomes. In yet another embodiment of the method, each bacterial cell of the population of bacterial cells expresses Pup. In a particular embodiment, the bacteria are a Mycobacterium species, including, but not limited to, M. tuberculosis and M. leprae.

In a further embodiment, each bacterial cell of the population of bacterial cells expresses a tagged form of Pup comprising Pup and a tag.

The present invention also includes methods for screening to identify de-pupylating enzymes. Pupylated substrates identified using the methods of the present invention may be used as tools for such screening methods, which may be performed using similar methods to those described above with respect to screening for activation and conjugation enzymes of Pup. Moreover, identifying modulators of de-pupylating enzymes is a promising approach for the development of therapeutic agents for the treatment of diseases associated with bacterial infections.

The present invention also encompasses a kit for expressing and purifying a fusion protein in a eukaryotic cell, the method comprising:

(a) expressing the fusion protein in the eukaryotic cell, wherein the fusion protein comprises a polypeptide sequence fused in frame to a Pup sequence; and
(b) purifying the fusion protein by affinity purification.

In one embodiment, the fusion protein further comprises an epitope tag and is purified by affinity purification of the epitope tag. Exemplary epitope tags useful for affinity purification, include, but are not limited to, histidine tags, FLAG tags, and streptavidin tags. Alternatively, the Pup sequence may be used for affinity purification.

In a particular embodiment, the polypeptide is fused to the Pup sequence via a linker. In a more particular embodiment, the linker comprises a cleavage site for a protease. Cleavage by the specific protease at the cleavage site thus enables release of the affinity purified polypeptide from the Pup sequence.

In an alternate embodiment, the polypeptide of the fusion protein is released from the Pup sequence by using a de-pupylating enzyme identified using the methods of the present invention.

Other features and advantages of the invention will be apparent from the following description of the preferred embodiments thereof, and from the claims.

### BRIEF DESCRIPTION OF THE DRAWINGS

FIG. 1A-D. Mtb Pup interacts with the ATPase Mpa and the proteasome substrate FabD. (A) Mpa interacts with Pup in an E. coli two-hybrid system. E. coli (cya) was transformed with combinations of plasmids encoding either of the two domains of B. pertussis Cya, T25 (“plasmid 1”) or T18 (“plasmid 2”), fused to Pup, Mpa, or no other protein (for plasmid details see FIG. 5 and Table 1). ‘Pup represents the 26 amino acid fragment identified from an Mtb genomic T25 library using T18C-Mpa as bait (i). Interactions that reconstituted functional Cya permitted growth on minimal lactose agar (right, “+”). All strains grew on minimal glucose agar (FIG. 5A). (B) Mpa interacts with Pup in vitro. Ni-NTA agarose was incubated with purified His₆-Pup, SigE-His₆ or E. coli “vector only” lysate, then each was incubated with recombinant Mpa (“input”). Input, flow-through, and elution fractions were separated by 15% SDS-PAGE and visualized with Coomassie Brilliant Blue (CBB). The same samples were analyzed by anti-Mpa immunoblot (IB, below). (C) Pup interacts with FabD in an Msm two-hybrid system. Msm was transformed with combinations of plasmids encoding either of the two domains of mDHFR, F(1,2) (“plasmid 1”) or F(3) (“plasmid 2”), fused to Pup, FabD, GCN4 (a Saccharomyces cerevisiae leucine zipper domain) or no other protein (for plasmid details see FIG. 5B and Table 1). Interactions that reconstituted functional mDHFR permitted growth on trimethoprim (Trim) (right, “+”). All strains grew on media lacking Trim (FIG. 5B). (D) Pup forms a stable complex with FabD in Msm. Equal amounts of lysates from Msm with plasmids encoding FLAG-FabD and either empty vector or His₆-Pup were incubated with anti-FLAG M2 affinity matrix for enrichment of FLAG-tagged proteins. Untagged FabD was the negative control. Samples were separated by 12% SDS-PAGE, and analyzed by anti-FLAG (left) or anti-His₅ (right) immunoblotting. FLAG-FabD migrates at the predicted size (arrow, left panel); the ˜45 kD anti-His₅-reactive protein (asterisk, right) is only seen in mycobacteria making both FLAG-FabD and His₆-Pup.

FIG. 2A-C. The C-terminus of Pup covalently attaches to Lys 173 of FabD. (A) Alignment of the C-terminus of Pup to that of Pup or ubiquitin from representative Actinomycetes or eukaryotes, respectively. Identical amino acids are shaded black. Sequences were compiled from the NCBI server and aligned using ClustalW (Chenna et al., Nucleic Acids Res 31, 3497 (Jul. 1, 2003)). The identical amino acid sequences shown for M. tuberculosis, M. smegmatis, M. leprae, and Rhodococcus are designated SEQ ID NO: 91; the amino acid sequence shown for C. jeikeium is designated SEQ ID NO: 92; and the identical amino acid sequences shown for yeast and human are designated SEQ ID NO: 93. (B) Purification of the FabD˜Pup complex. Msm was co-transformed with plasmids encoding FLAG-FabD and His₆-Pup. His₆-Pupylated proteins were purified with Ni-NTA agarose, then passed over an anti-FLAG M2 affinity matrix to enrich for FLAG-FabD˜His₆-Pup. Proteins from each purification step were analyzed by 12% SDS-PAGE and visualized with CBB. (C) Tandem mass (MS/MS) spectrum of a FabD tryptic peptide derived by collision-induced dissociation of the (M+2H)²⁺ precursor, m/z 869.963 (1.55 ppm). Singly-charged fragment ions marked in the spectrum represent peptide bond cleavage resulting in the sequence information recorded from both the N and C termini (b- and y-type ions, respectively). This spectrum, searched with the SEQUEST program, matched to the peptide shown with a mass shift corresponding to a de-amidation event, converting the Pup C-terminal Gln (Q*) to Glu. High mass accuracy MS/MS unambiguously confirms covalent modification of lysine in FLAG-FabD by His₆-Pup, with multiple matching b- and y-type ions. Additional detailed fragment ion information and additional spectra are presented in FIG. 9. The amino acid sequence of the peptide shown is designated SEQ ID NO: 94.

FIG. 3A-C. Pupylation is associated with Mtb proteasome substrates. (A) Aberrant pupylation levels correlate with proteasome-defective states. Equal amounts of soluble Mtb lysates from WT, mpa and pafA strains were incubated with Ni-NTA for enrichment of FLAG-FabD-His₆. Samples were deliberately over-loaded to detect pupylated protein and observe the relative amounts of unpupylated versus pupylated FabD. Anti-FLAG immunoblots (left) of Ni-NTA eluates detected both unpupylated (arrow) and pupylated (asterisk) FLAG-FabD-His₆. Anti-Pup immunoblots (right) of the same samples detected only Pup˜FLAG-FabD-His₆ (asterisk) in WT and mpa Mtb but not the pafA strain. (B) Pupylation of FabD is specific. FLAG-DlaT-His₆ was purified from WT Mtb. Anti-FLAG immunoblots (left) of eluates detected a protein at the predicted size of FLAG-DlaT-His₆, but no pupylated species was detected (right). This blot was part of the same membrane analyzed with anti-Pup in part (A). Ponceau S staining shows protein on this membrane (FIG. 10). (C) Multiple pupylated proteins are present in Mtb, but not in a pafA mutant. Anti-Pup immunoblots of Mtb lysates from WT, mpa, pafA, pafB, and pafC strains. Several proteins appeared to accumulate in the mpa mutant (arrows). Equivalent cell numbers were analyzed and the same blot was used for detection of endogenous DlaT (loading control). All samples were separated by 12% SDS-PAGE.

FIG. 4. Proposed model of substrate degradation by the Mtb proteasome. The C-terminus of Pup may be de-amidated either before or during its conjugation to substrate lysines. PafA may play a role in the conjugation of Pup. Mpa may then recognize the pupylated substrate through non-covalent interactions with Pup. The substrate could then be unfolded and threaded into the proteasome core where it is degraded. It is unknown if Pup is also degraded, or if it is removed and recycled.

FIG. 5A-B. BTH controls. (A) All E. coli BTH strains grew on minimal E-salts agar supplemented with 1% glucose. (B) All Msm BTH strains grew on 7H11 lacking Trim.

FIG. 6. Genomic orientation of pup in Mtb. pup (Rv2111c) is upstream of the proteasome core genes prcB (Rv2110c) and prcA (Rv2109c). The stop and start codons of pup/prcB overlap, suggesting that they are co-transcribed and translationally coupled. Sequences were compiled from the NCBI server.

FIG. 7. His₆-Pup˜FLAG-FabD complex is not formed in E. coli. Equal amounts of soluble E. coli lysates from WT strains co-expressing FLAG-FabD and either His₆-Pup or empty vector were incubated with anti-FLAG M2 affinity matrix for enrichment of FLAG-tagged proteins. Samples were analyzed by 12% SDS-PAGE. Anti-FLAG immunoblots (left) of eluates recognized a protein at the predicted size of FLAG-FabD (arrow). Anti-His₆ immunoblots (right) of the same samples failed to detect a His₆-Pup˜FLAG-FabD complex.

FIG. 8. Homologues of Pup that naturally terminate in glutamate. Alignment of the C-terminus of Pup to the C-termini of Pup from representative Actinomycetes. The conserved amino acids are shaded black. Sequences were compiled from the NCBI server and aligned using ClustalW (Chenna et al., Nucleic Acids Res 31, 3497 (Jul. 1, 2003)). The identical amino acid sequences shown for M. tuberculosis, M. avium, M. smegmatis, M. leprae, and Rhodococcus are designated SEQ ID NO: 91; the amino acid sequence shown for Streptomyces coelicoloris is designated SEQ ID NO: 95; the amino acid sequence shown for Nocardioides is designated SEQ ID NO: 96; the amino acid sequence shown for Corynebacterium glutamicum is designated SEQ ID NO: 97; the amino acid sequence shown for Acidothermus cellulolyticus is designated SEQ ID NO: 98; and the amino acid sequence shown for Frankia alni is designated SEQ ID NO: 99.

FIG. 9A-C. Evidence for isopeptide linkage between a FabD lysine and the C-terminus of Pup. The modified fragment ion nomenclature used here includes the charge (either + or 2+), subscript 1 or 2, referring to the FabD and Pup peptides respectively, and the sequential fragment number. The capitalized B/Y refers to fragment ions that include the isopeptide bond. (A) Detailed assignment of both singly- and doubly-charged b- and y-type ion fragments for the spectrum shown in FIG. 2C in the main text. Mass deviation information is included for ions matching within 10 ppm. The amino acid sequence of the peptide shown is designated SEQ ID NO: 94. (B) Tandem mass (MS/MS) spectrum of the same pupylated peptide as shown in FIG. 2C in the main text and derived by collision-induced dissociation of the (M+3H)³⁺ precursor, m/z 580.312 (0.60 ppm). (C) Tandem mass (MS/MS) spectrum of a FabD partially tryptic peptide derived from collision-induced dissociation of the (M+2H)²⁺ precursor, m/z 673.344 (2.13 ppm). The peptide arises from cleavage at the weak Asp-Pro peptide bond.

FIG. 10 shows Ponceau S staining of the nitrocellulose membrane analyzed by anti-Pup immunoblotting in FIG. 3(A, B). Each lane contained the respective purified His₆-tagged proteins. All samples were separated by 12% SDS-PAGE.

FIG. 11 shows an amino acid sequence of Pup (SEQ ID NO: 2) encoded by SEQ ID NO: 1.

FIG. 12 shows Table 1 which lists antibodies, plasmids, and primers used herein.

FIG. 13 shows an immunoblot probed with anti-Pup monoclonal antibody 92 (mAb 92). The mAb 92 immunoblot reveals the presence of multiple pupylated proteins in Mtb, but not in a pafA mutant. Equivalent cell numbers were analyzed in each lane. All samples were separated by 10% SDS-PAGE.

FIG. 14 shows Table 2 which presents a list of the peptides identified using the Pup affinity purification protocol described herein.

FIG. 15 shows Table 3 which presents a list of peptides to which Pup is covalently attached as detected by changes in tryptic fragment mass.

### DETAILED DESCRIPTION OF THE INVENTION

In order to more clearly set forth the parameters of the present invention, the following definitions are used:

As used in this specification and the appended claims, the singular forms “a”, “an”, and “the” include plural references unless the context clearly dictates otherwise. Thus for example, reference to “the method” includes one or more methods, and/or steps of the type described herein and/or which will become apparent to those persons skilled in the art upon reading this disclosure.

The term “complementary” refers to two DNA strands that exhibit substantial normal base pairing characteristics. Complementary DNA may, however, contain one or more mismatches.

The term “hybridization” refers to the hydrogen bonding that occurs between two complementary DNA strands.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it is generally associated in its natural state (i.e., in cells or tissues). An isolated nucleic acid (either DNA or RNA) may further represent a molecule produced directly by biological or synthetic means and separated from other components present during its production.

“Natural allelic variants”, “mutants” and “derivatives” of particular sequences of nucleic acids refer to nucleic acid sequences that are closely related to a particular sequence but which may possess, either naturally or by design, changes in sequence or structure. By closely related, it is meant that at least about 60%, but often, more than 85%, of the nucleotides of the sequence match over the defined length of the nucleic acid sequence referred to using a specific SEQ ID NO. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes, such as to change an amino acid codon or sequence in a regulatory region of the nucleic acid. Such specific changes may be made in vitro using a variety of mutagenesis techniques or produced in a host organism placed under particular selection conditions that induce or select for the changes. Such sequence variants generated specifically may be referred to as “mutants” or “derivatives” of the original sequence.

The terms “percent similarity”, “percent identity” and “percent homology” when referring to a particular sequence are used as set forth in the University of Wisconsin GCG software program and are known in the art.

The present invention also includes active portions, fragments, derivatives and functional or non-functional mimetics of a Pup of the invention. An “active portion” of a Pup means a peptide that is less than the full length Pup, but which retains measurable biological activity.

A “fragment” or “portion” of a Pup means a stretch of amino acid residues of at least about five to seven contiguous amino acids, often at least about seven to nine contiguous amino acids, typically at least about nine to thirteen contiguous amino acids and, most preferably, at least about twenty to thirty or more contiguous amino acids. A “derivative” of the Pup or a fragment thereof means a polypeptide modified by varying the amino acid sequence of the protein, e.g. by manipulation of the nucleic acid encoding the protein or by altering the protein itself. Such derivatives of the natural amino acid sequence may involve insertion, addition, deletion or substitution of one or more amino acids, and may or may not alter the essential activity of the original Pup.

Different “variants” of the Pup may exist in nature. These variants may be alleles characterized by differences in the nucleotide sequences of the gene coding for the protein, or may involve different RNA processing or post-translational modifications. The skilled person can produce variants having single or multiple amino acid substitutions, deletions, additions or replacements. These variants may include inter alia: (a) variants in which one or more amino acids residues are substituted with conservative or non-conservative amino acids, (b) variants in which one or more amino acids are added to the Pup, (c) variants in which one or more amino acids include a substituent group, and (d) variants in which the Pup is fused with another peptide or polypeptide such as a fusion partner, a protein tag or other chemical moiety, that may confer useful properties to Pup, such as, for example, an epitope for an antibody, a polyhistidine sequence, a biotin moiety and the like. Other Pups of the invention include variants in which amino acid residues from one species are substituted for the corresponding residue in another species, either at conserved or non-conserved positions. In another embodiment, amino acid residues at non-conserved positions are substituted with conservative or non-conservative residues. The techniques for obtaining these variants, including genetic (suppressions, deletions, mutations, etc.), chemical, and enzymatic techniques are known to a person having ordinary skill in the art.

To the extent such allelic variations, analogues, fragments, derivatives, mutants, and modifications, including alternative nucleic acid processing forms and alternative post-translational modification forms result in derivatives of Pup that retain any of the biological properties of Pup, they are included within the scope of this invention.

The term “ortholog” as used herein refers to polypeptides encoded by nucleic acid sequences of a different species whose polypeptide product has greater than 60% identity to a Pup encoding sequence and/or whose gene products have similar three dimensional structure and/or biochemical activities of Pup. The use of such orthologs in the methods of the invention is contemplated herein.

The term “homolog” as used herein refers to polypeptides encoded by nucleic acid sequences of the same species whose polypeptide product has greater than 60% identity to a Pup encoding sequence and/or whose gene products have similar three dimensional structure and biochemical activities of Pup.

The term “functional” as used herein implies that the nucleic or amino acid sequence is functional for the recited assay or purpose.

The term “functional fragment” as used herein implies that the nucleic or amino acid sequence is a portion or subdomain of a full length polypeptide and is functional for the recited assay or purpose.

The phrase “consisting essentially of” when referring to a particular nucleotide or amino acid means a sequence having the properties of a given SEQ ID No:. For example, when used in reference to an amino acid sequence, the phrase includes the sequence per se and molecular modifications that would not affect the basic and novel characteristics of the sequence.

A “replicon” is any genetic element, for example, a plasmid, cosmid, bacmid, phage or virus, that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression vector” or “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the term “operably linked” refers to a regulatory sequence capable of mediating the expression of a coding sequence and which are placed in a DNA molecule (e.g., an expression vector) in an appropriate position relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of predetermined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield an primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Primers may be labeled fluorescently with 6-carboxyfluorescein (6-FAM). Alternatively primers may be labeled with 4, 7, 2′, 7′-Tetrachloro-6-carboxyfluorescein (TET). Other alternative DNA labeling methods are known in the art and are contemplated to be within the scope of the invention.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like). “Mature protein” or “mature polypeptide” shall mean a polypeptide possessing the sequence of the polypeptide after any processing events that normally occur to the polypeptide during the course of its genesis, such as proteolytic processing from a polypeptide precursor. In designating the sequence or boundaries of a mature protein, the first amino acid of the mature protein sequence is designated as amino acid residue 1.

The term “tag”, “tag sequence” or “protein tag” refers to a chemical moiety, either a nucleotide, oligonucleotide, polynucleotide or an amino acid, peptide or protein or other chemical, that when added to another sequence, provides additional utility or confers useful properties to the sequence, particularly with regard to methods relating to the detection or isolation of the sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules [e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, streptavidin (“Strep” tag) and the like] may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by, the trained artisan, and are contemplated to be within the scope of this definition.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. In other applications, the introduced nucleic acid may exist in the recipient cell or host organism only transiently.

A “clone” or “clonal cell population” is a population of cells derived from a single cell or common ancestor by mitosis or fission (with respect to bacteria).

A “cell line” is a clone of a primary cell or cell population that is capable of stable growth in vitro for many generations.

The compositions containing the molecules or compounds of the invention can be administered for diagnostic and/or therapeutic treatments. In therapeutic applications, compositions are administered to a patient already suffering from a disease or condition caused by or associated with a bacterial infection (such as, e.g., tuberculosis or leprosy) in an amount sufficient to cure or at least partially arrest the symptoms of the disease and its complications. An amount adequate to accomplish this is defined as a “therapeutically effective amount or dose.” Amounts effective for this use will depend on the severity of the disease and the weight and general state of the patient.

An “immune response” signifies any reaction produced by an antigen, such as a protein antigen, in a host having a functioning immune system. Immune responses may be either humoral, involving production of immunoglobulins or antibodies, or cellular, involving various types of B and T lymphocytes, dendritic cells, macrophages, antigen presenting cells and the like, or both. Immune responses may also involve the production or elaboration of various effector molecules such as cytokines, lymphokines and the like. Immune responses may be measured both in in vitro and in various cellular or animal systems.

An “antibody” or “antibody molecule” is any immunoglobulin, including antibodies and fragments thereof, that binds to a specific antigen. The term includes polyclonal, monoclonal, chimeric, and bispecific antibodies. As used herein, antibody or antibody molecule contemplates both an intact immunoglobulin molecule and an immunologically active portion of an immunoglobulin molecule such as those portions known in the art as Fab, Fab′, F(ab′)2 and F(v).

As used herein, an “agent”, “candidate compound”, or “test compound” may be used to refer to, for example, nucleic acids (e.g., DNA and RNA), carbohydrates, lipids, proteins, peptides, peptidomimetics, small molecules and other drugs.

The term “control substance”, “control agent”, or “control compound” as used herein refers a molecule that is inert or has no activity relating to an ability to modulate a biological activity. With respect to the present invention, such control substances are inert with respect to an ability to modulate a Pup activity, and/or a signaling pathway that contributes to an activity of Pup, e.g., a proteasome degradation pathway. Exemplary controls include, but are not limited to, solutions comprising physiological salt concentrations.

The term “Pup modulatory agent” as used herein refers to an agent that is capable of modulating (e.g., increasing or decreasing) an activity attributable to Pup. Methods for screening/identifying such agents are presented herein below.

As used herein, the term “proteasome substrate” is used to refer to polypeptides that are recognized and targeted for degradation by the proteasome. Proteasomes are large protein complexes found in all eukaryotes and archaea, as well as some bacteria. The main function of the proteasome is to degrade unneeded or damaged proteins by proteolysis, a chemical reaction that breaks protein bonds. Proteasomes are major components of the cellular machinery that enables cells to regulate the concentration of particular proteins and degrade misfolded proteins.

As used herein, the phrase “conjugated to proteasome substrates” refers to the covalent attachment of Pup to substrates. As exemplified herein, the covalent attachment of Pup to substrates may occur via an N-terminal peptide or isopeptide attachment of Pup to substrates. As further shown herein, covalent conjugation or attachment of Pup to a substrate serves to target the Pup-conjugated substrate for proteasome-mediated degradation. Conjugation to Pup is, therefore, associated with substrate degradation. With regard to Pup˜Pup, Pup has lysines that could potentially be modified by other Pups in a manner analogous to ubiquitin.

Pup may also be involved in non-degradation associated modification, much like ubiquitin/SUMO have been shown to modulate protein localization and activity.

As used herein “a change in the population of polypeptides covalently conjugated to Pup” refers to an increase or decrease in the amount of a particular polypeptide covalently conjugated to Pup or a difference in the spectrum polypeptides conjugated to Pup. With regard to the spectrum of polypeptides conjugated to Pup, this refers to the collection of different polypeptides conjugated to Pup that differ with respect to their primary amino acid sequences.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and described the methods and/or materials in connection with which the publications are cited.

### ASPECTS OF THE INVENTION

Before the present discovery and methods of use thereof are described, it is to be understood that this invention is not limited to particular assay methods, or test compounds and experimental conditions described, as such methods and compounds may vary. It is also to be understood that the terminology used herein is for the purpose of describing particular embodiments only, and is not intended to be limiting, since the scope of the present invention will be limited only the appended claims.

The present inventors have discovered the first prokaryotic ubiquitin-like protein (Pup) to be identified. It is, moreover, noteworthy that proteasome-bearing prokaryotes were thought to degrade proteins via an ubiquitin-independent pathway prior to the instant discovery. Indeed, Pup is the first known post-translational protein modifier to be identified in bacteria. As described herein for the first time, Pup specifically conjugates to proteasome substrates in the pathogen Mycobacterium tuberculosis (Mtb). The identification of Pup facilitates the isolation and identification of prokaryotic proteasome substrates, which can be evaluated as suitable targets for therapeutics. The identification of Pup also provides reagents useful for screening assays directed to the identification of enzymes involved in the conjugation of Pup to substrates (pupylation) and enzymes required for de-pupylating (removal of conjugated Pup) for substrates. Moreover, methods and agents useful for modulating Pup activity and prokaryotic proteasome activity may be used to advantage as targeted therapeutics for the treatment of diseases associated with bacterial infections. More particularly, it is envisioned that agents identified using the method of the present invention may be useful for the treatment of patients with, for example, tuberculosis and leprosy.

Pupylated substrates identified using the method of the present invention may be used as components in screens to identify de-pupylating enzymes. In an embodiment of a method directed to a screen for identifying a de-pupylating enzyme, pupylated substrates are used in either cell-based or test-tube based screening assays.

A skilled practitioner would be aware that the literature provides details pertaining to numerous additional assays that may be used in conjunction with the present invention. One such reference is U.S. Pat. No. 7,282,491, the contents of which are incorporated herein in their entirety.

Accordingly, the invention is directed to an isolated nucleic acid sequence that encodes a polypeptide comprising SEQ ID NO: 2 or a functional fragment thereof. Also encompassed by the invention are expression vectors comprising an isolated nucleic acid sequence which encodes a polypeptide comprising SEQ ID NO: 2 or a functional fragment thereof. Cells comprising these expression vectors are also envisioned, as are transgenic animals comprising an isolated nucleic acid sequence of the invention, wherein a nucleic acid sequence is expressed in at least one cell of the transgenic animal.

In another aspect of the invention, an isolated amino acid sequence comprising a polypeptide of SEQ ID NO: 2 or a functional fragment thereof is presented. Also included are expression vectors encoding an amino acid sequence of the invention, wherein expression of the amino acid sequence is controlled by regulatory sequences in the expression vector, cells comprising such expression vectors, and transgenic animals comprising an amino acid sequence of the invention, wherein the amino acid sequence is expressed in at least one cell in the transgenic animal.

In another aspect of the invention, an isolated nucleic acid sequence comprising SEQ ID NO: 1, wherein the nucleic acid sequence encodes Pup or a functional fragment thereof, is presented. An expression vector comprising a nucleic acid sequence of SEQ ID NO: 1, wherein the nucleic acid sequence encodes Pup or a functional fragment thereof, and SEQ ID NO: 1 is operably linked to a regulatory sequence is also described. Moreover, a cell comprising such an expression vector comprising a nucleic acid sequence of SEQ ID NO: 1 is presented. In another aspect, a transgenic animal comprising a nucleic acid sequence comprising SEQ ID NO: 1, wherein the nucleic acid sequence encodes Pup or a functional fragment thereof, and wherein the nucleic acid sequence is expressed in at least one cell of the transgenic animal, is presented.

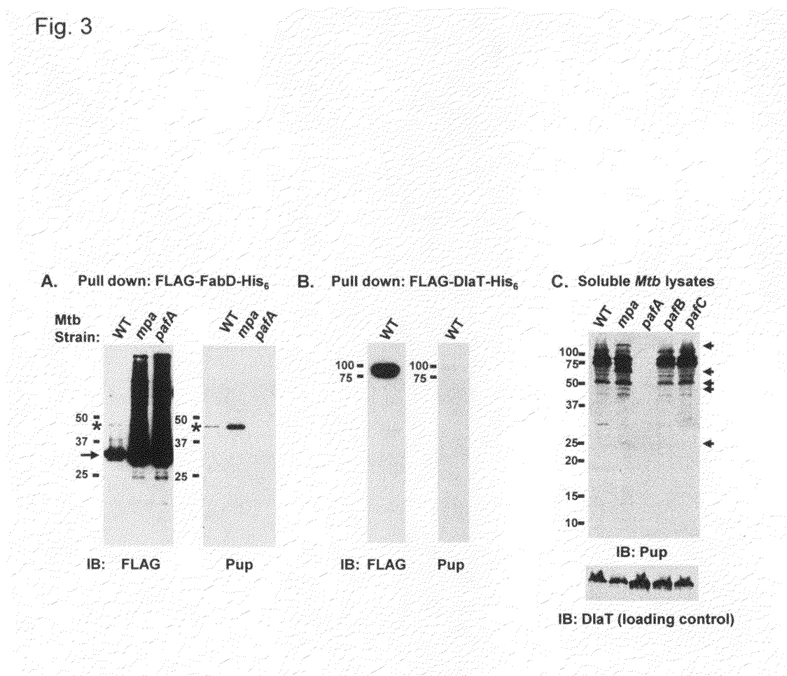

The present invention also describes antibodies that are immunologically specific for Pup. The Pup antibodies described herein are the first antibodies specific for Pup to be characterized. As described in detail herein below, polyclonal antibodies immunologically specific for Pup have been raised in rabbits in response to histidine tagged-full length Pup (see Example I and FIG. 3). As shown herein, affinity purified polyclonal rabbit antisera raised against the His₆-Pup fusion protein recognizes pupylated substrates on immunoblots of Mtb lysates (FIG. 3).

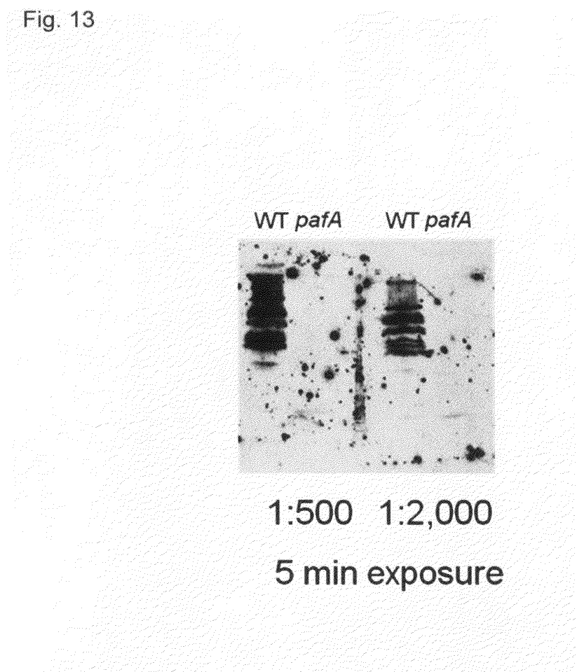

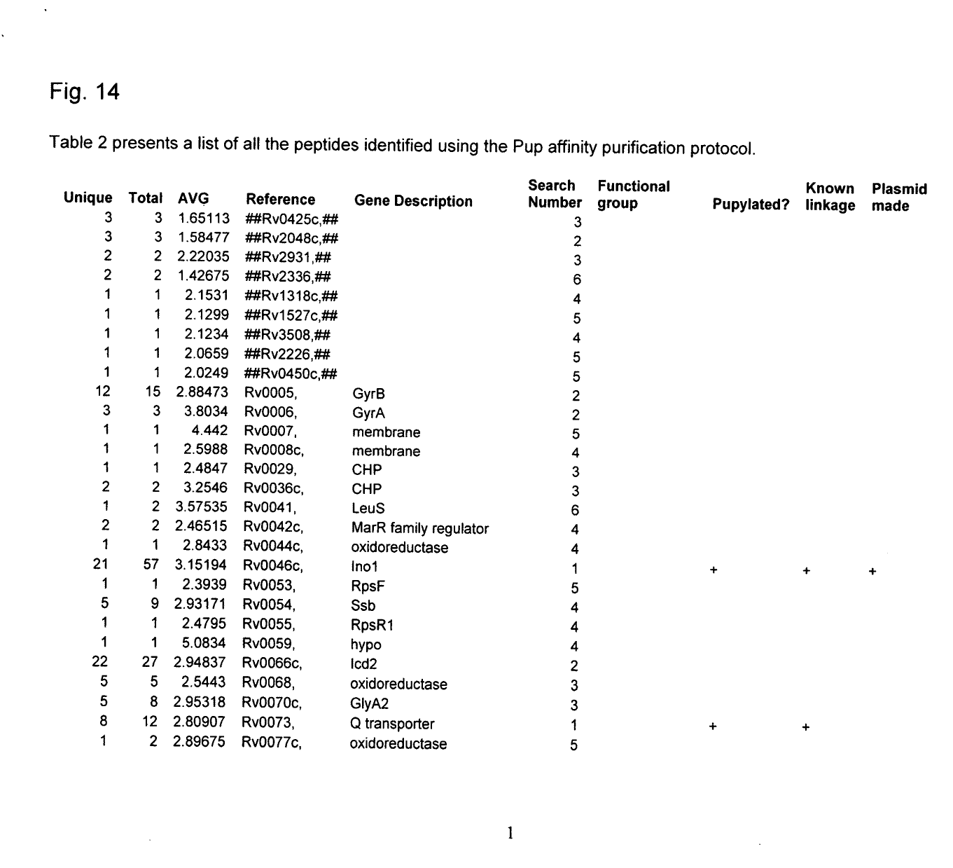

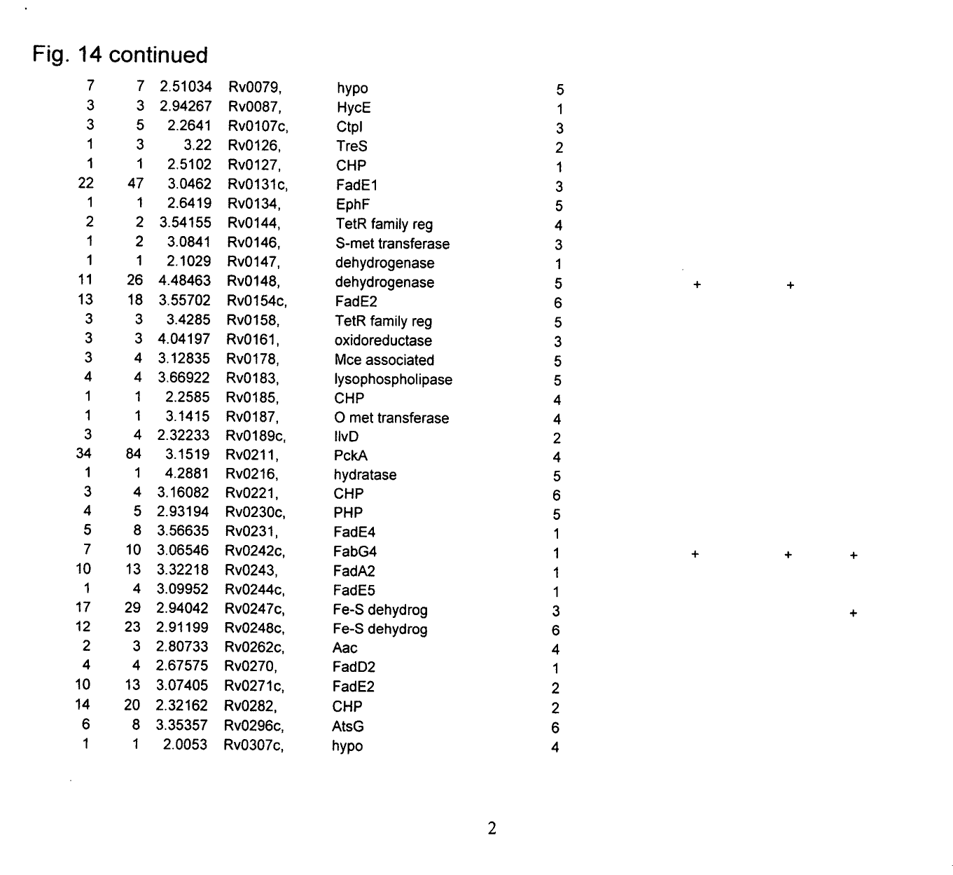

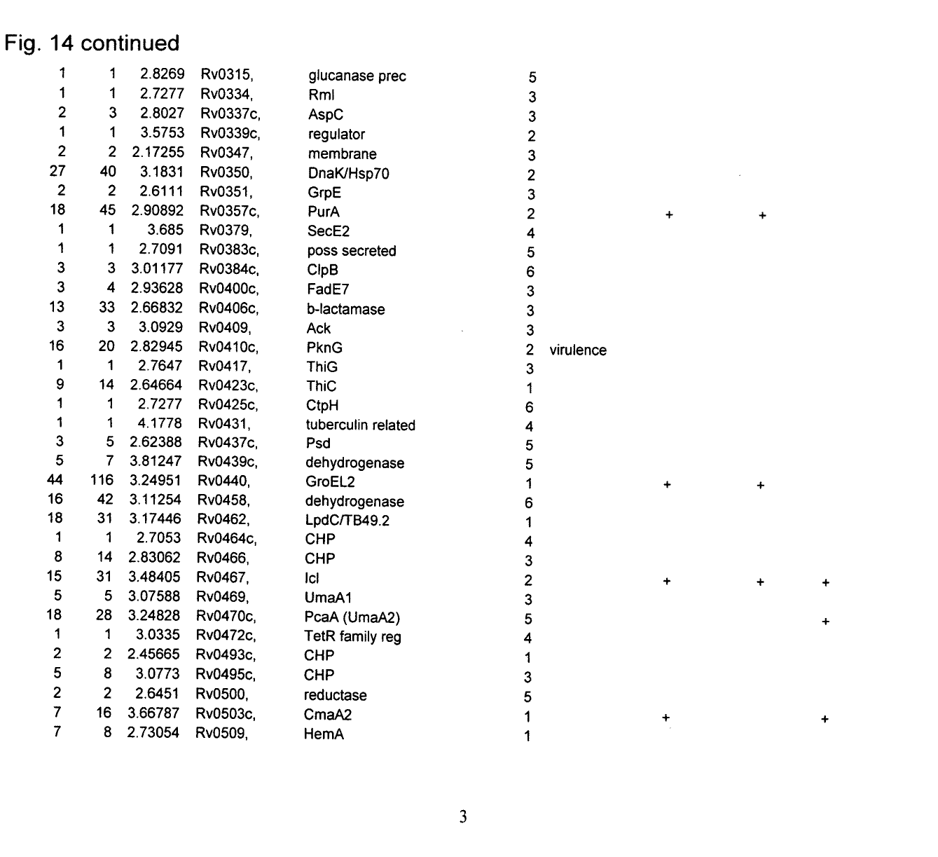

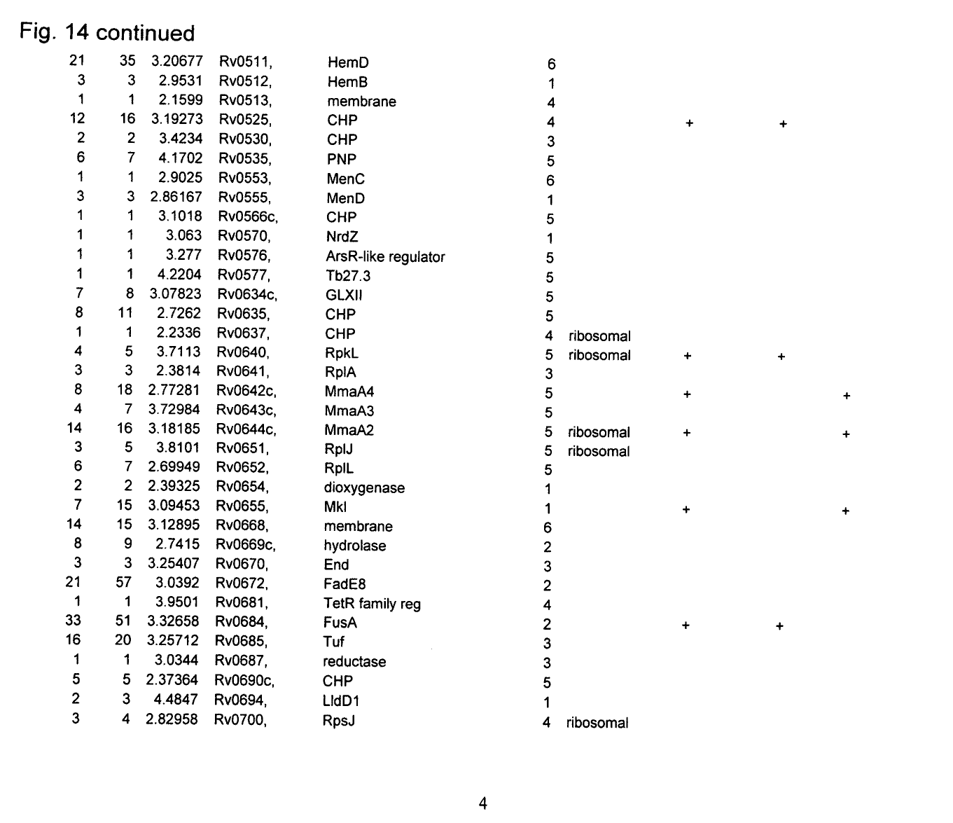

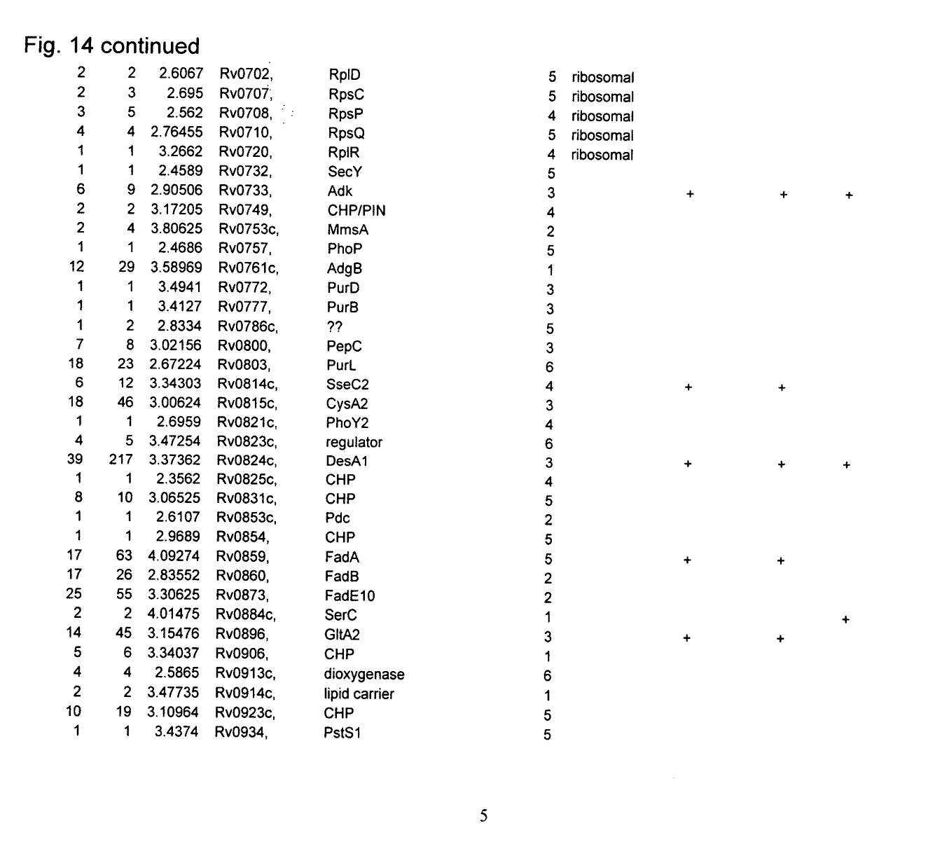

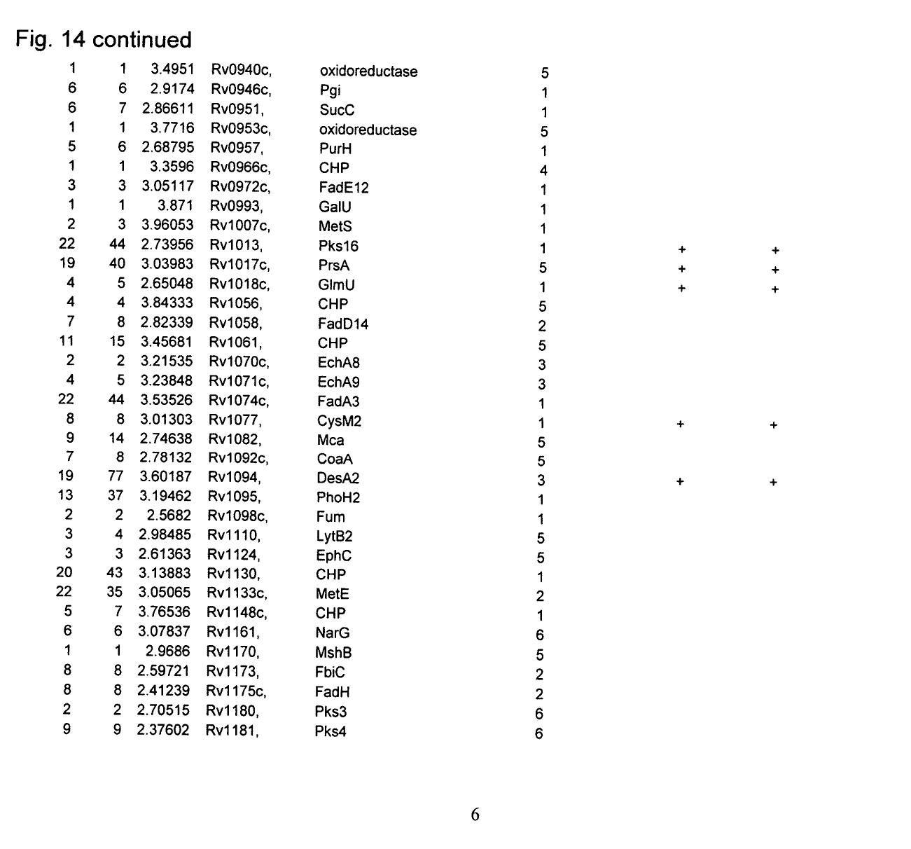

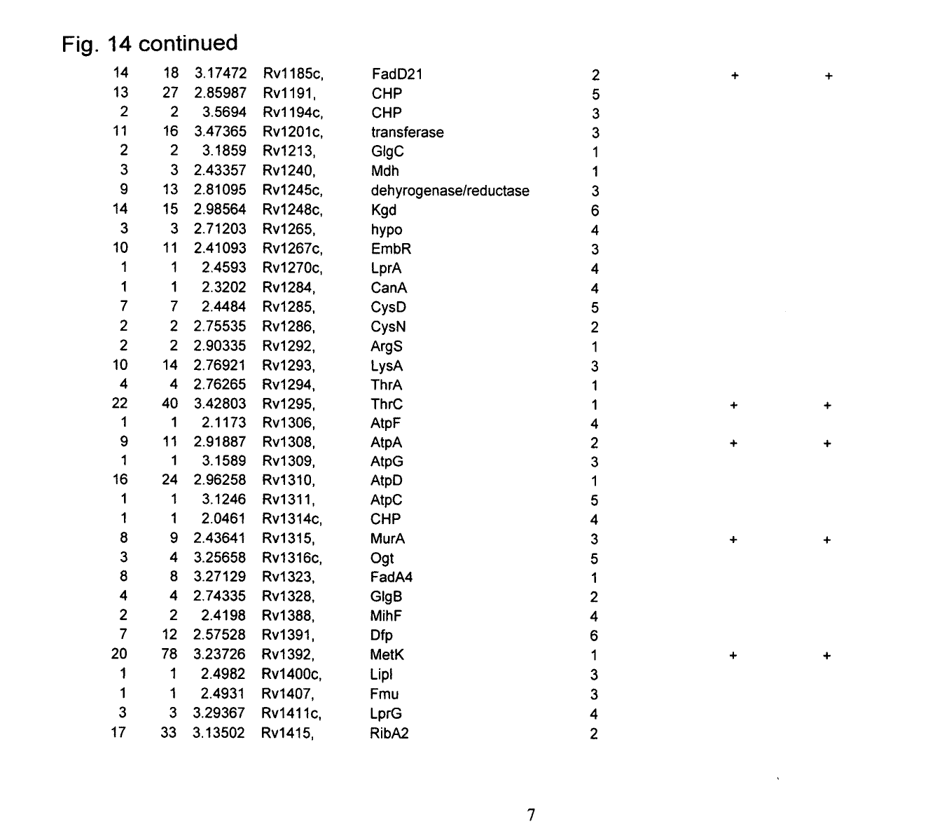

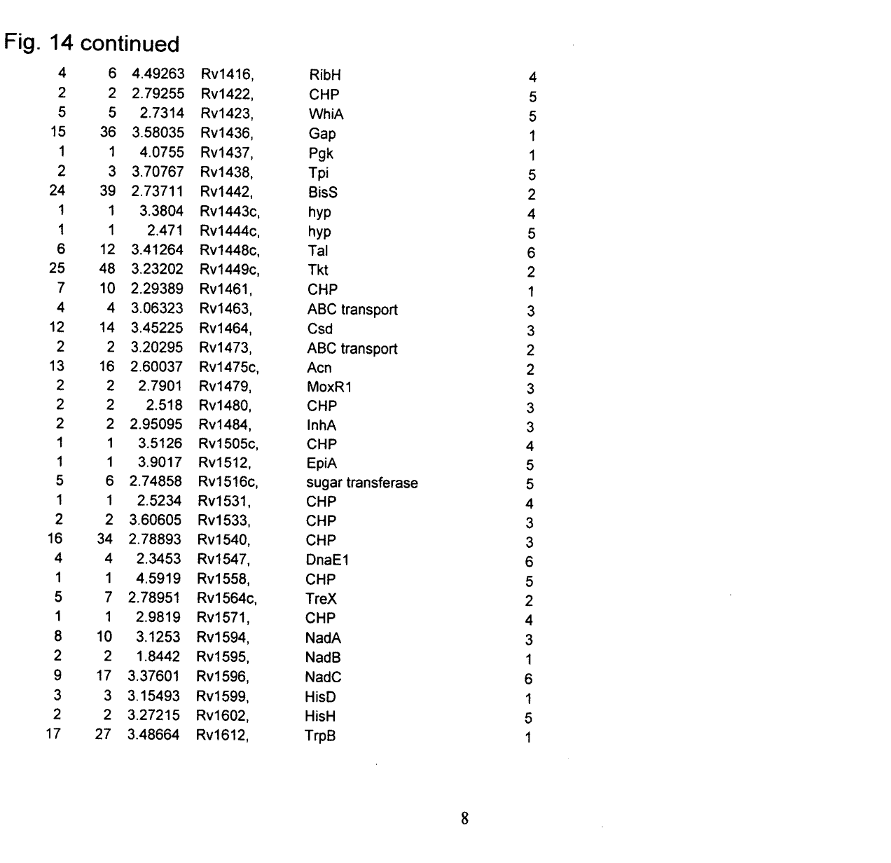

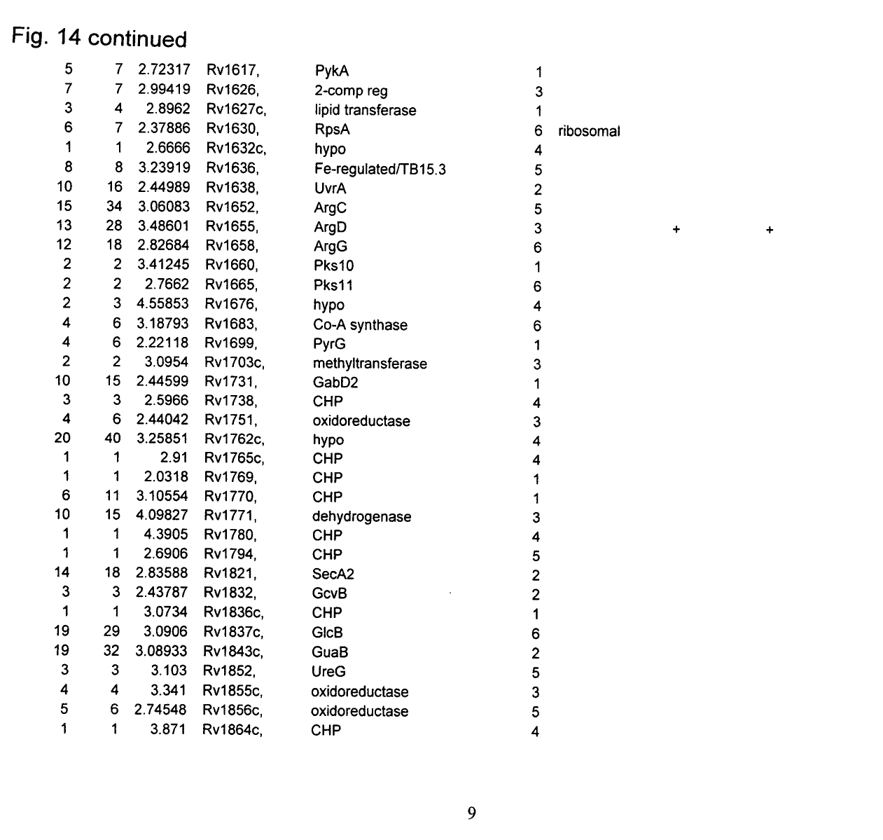

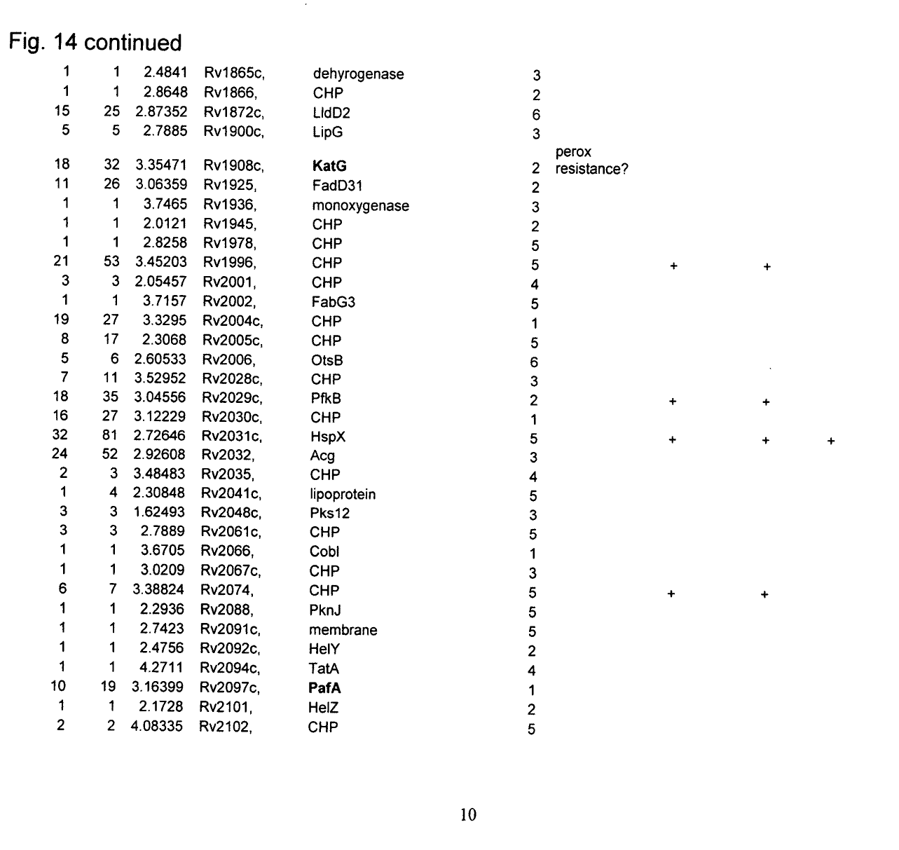

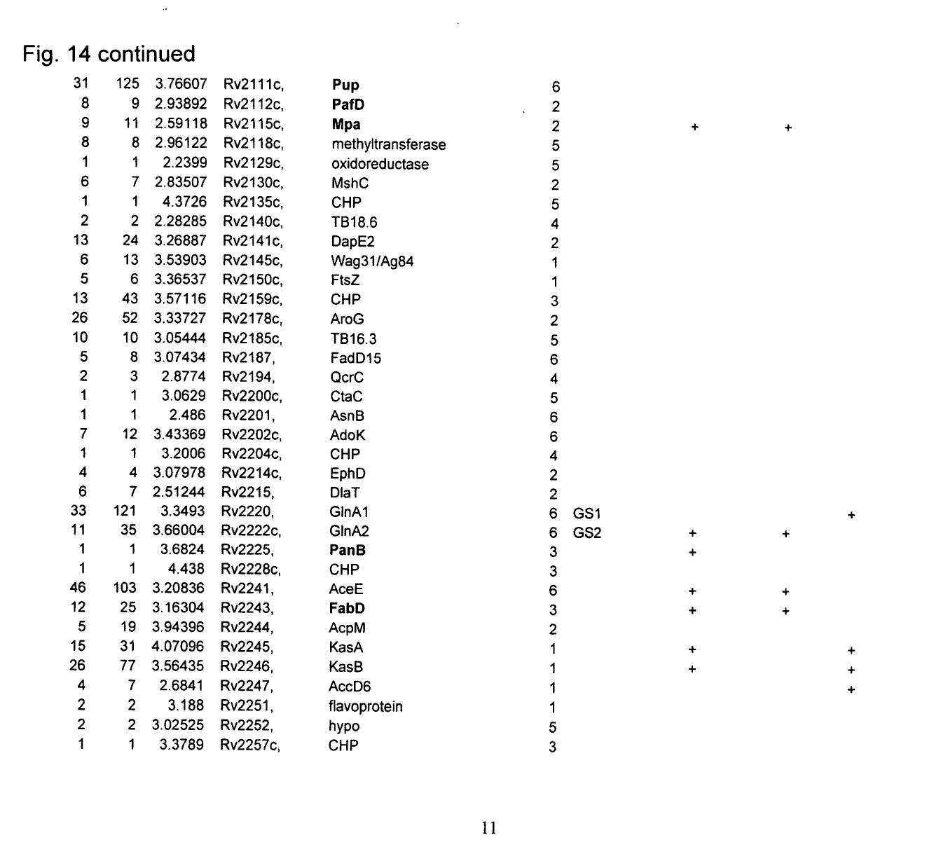

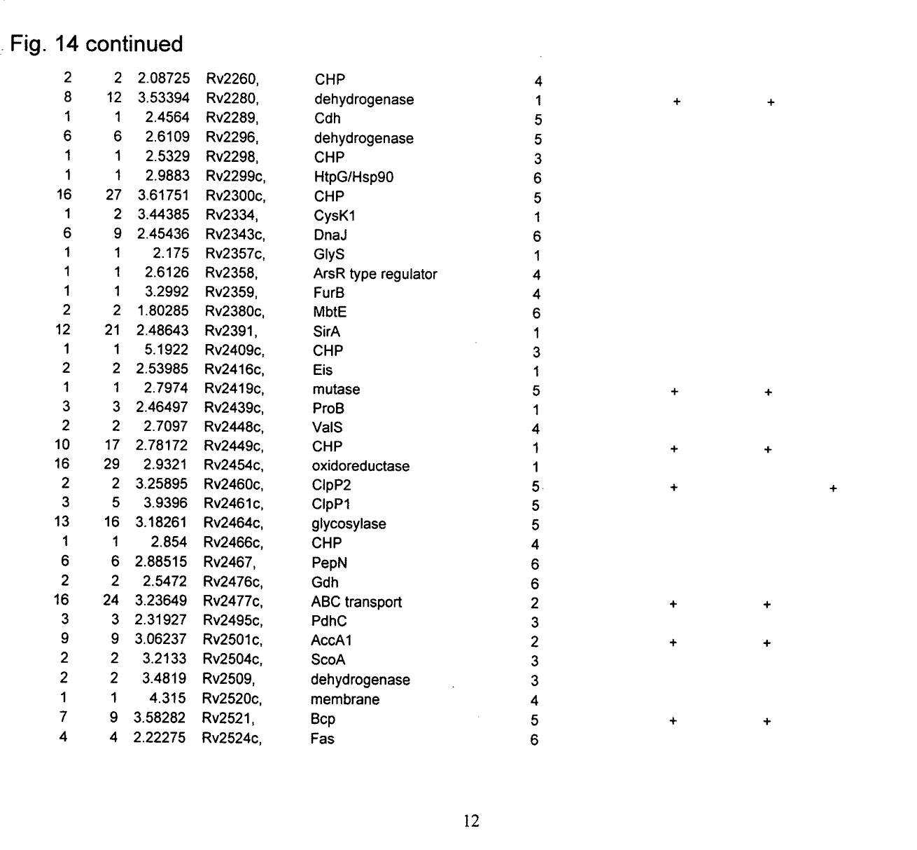

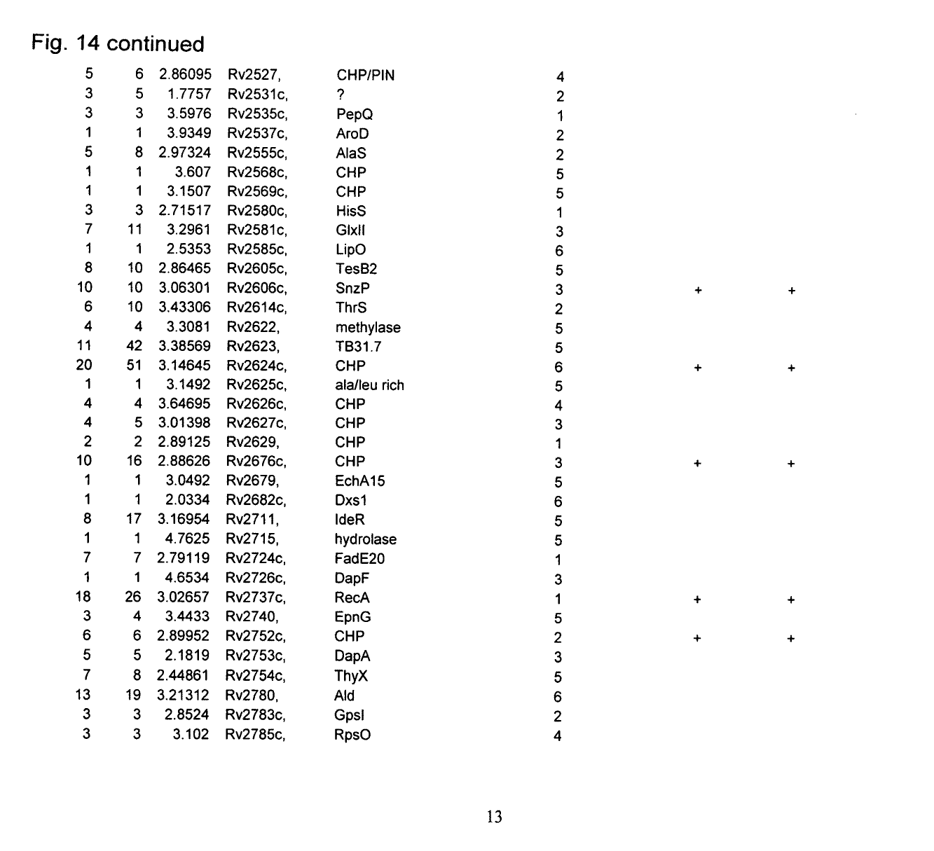

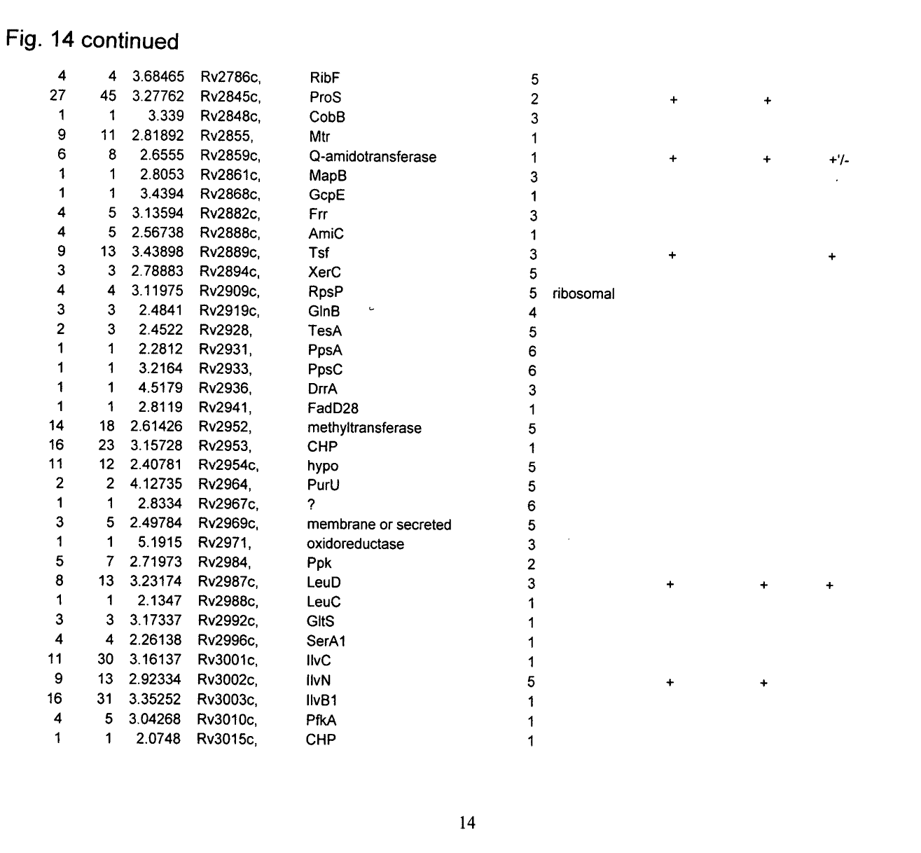

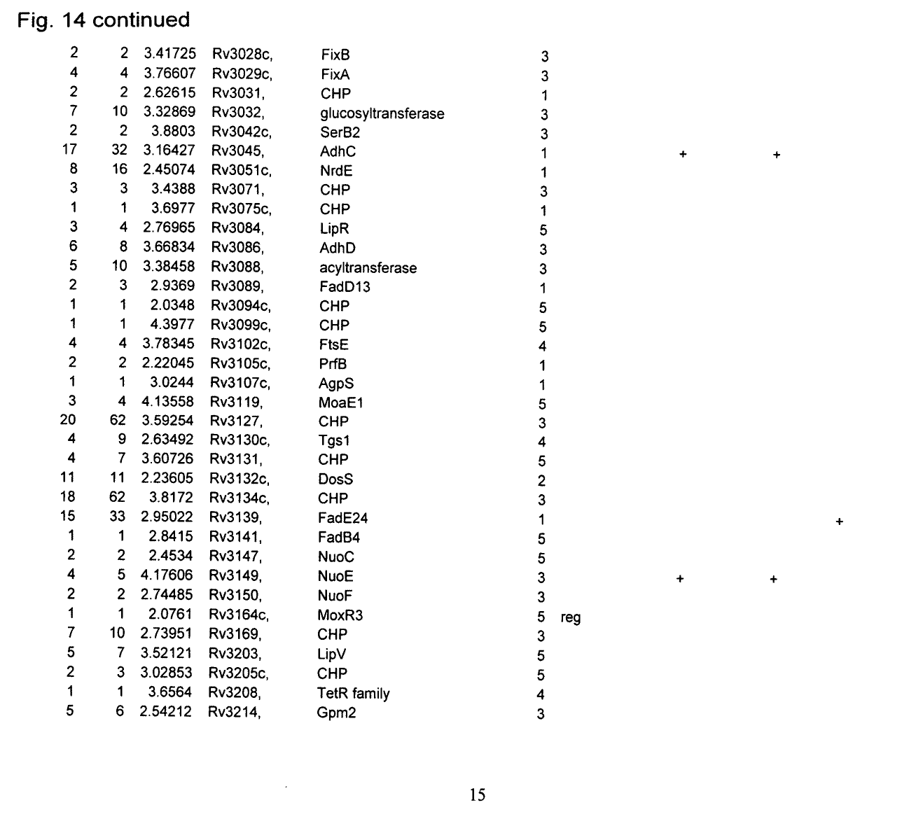

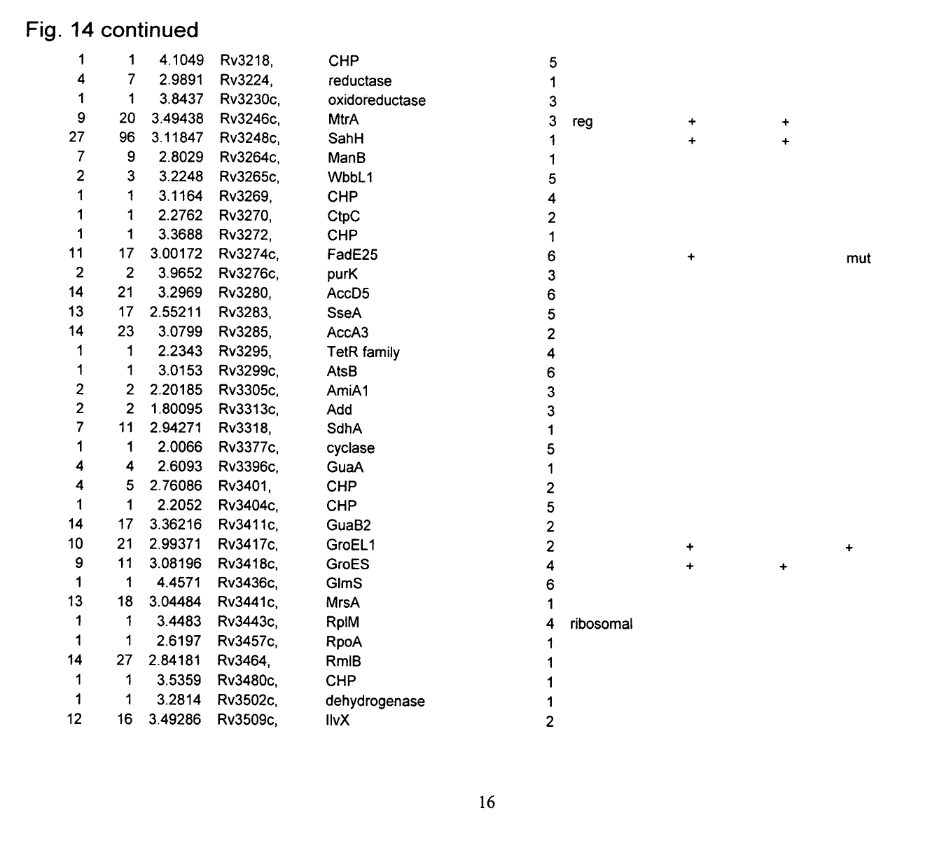

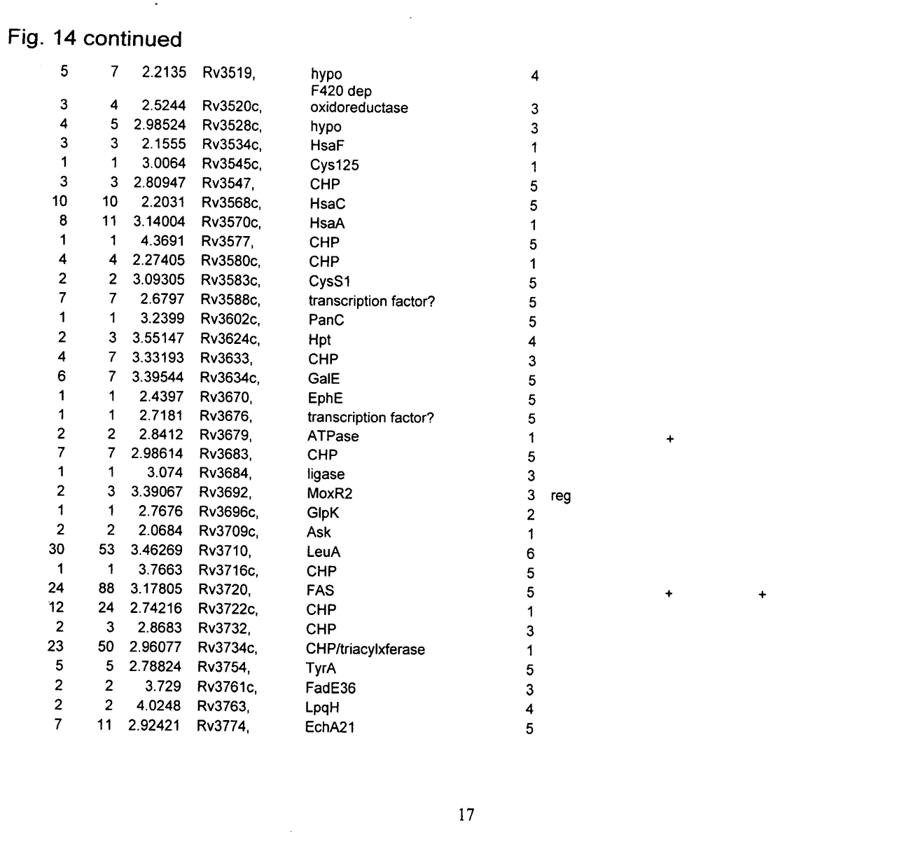

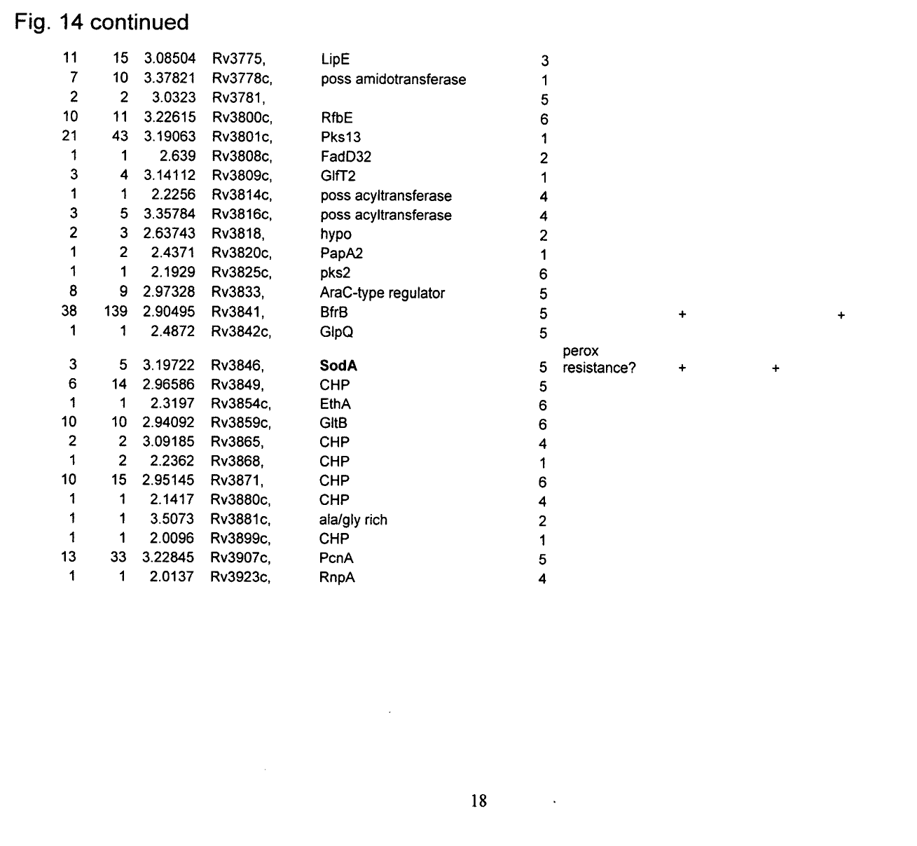

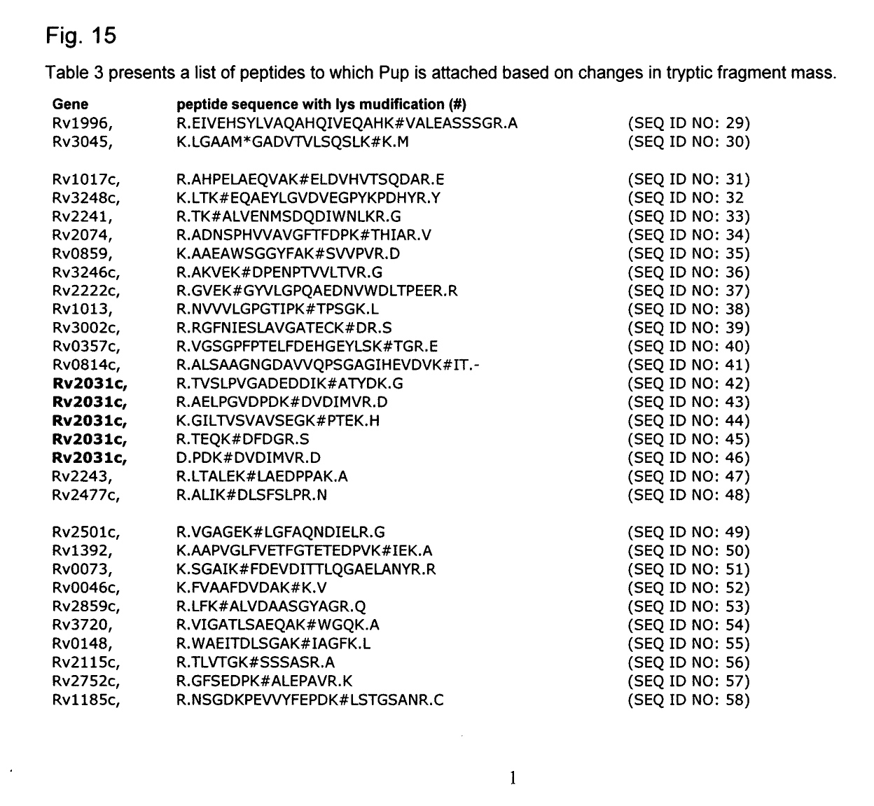

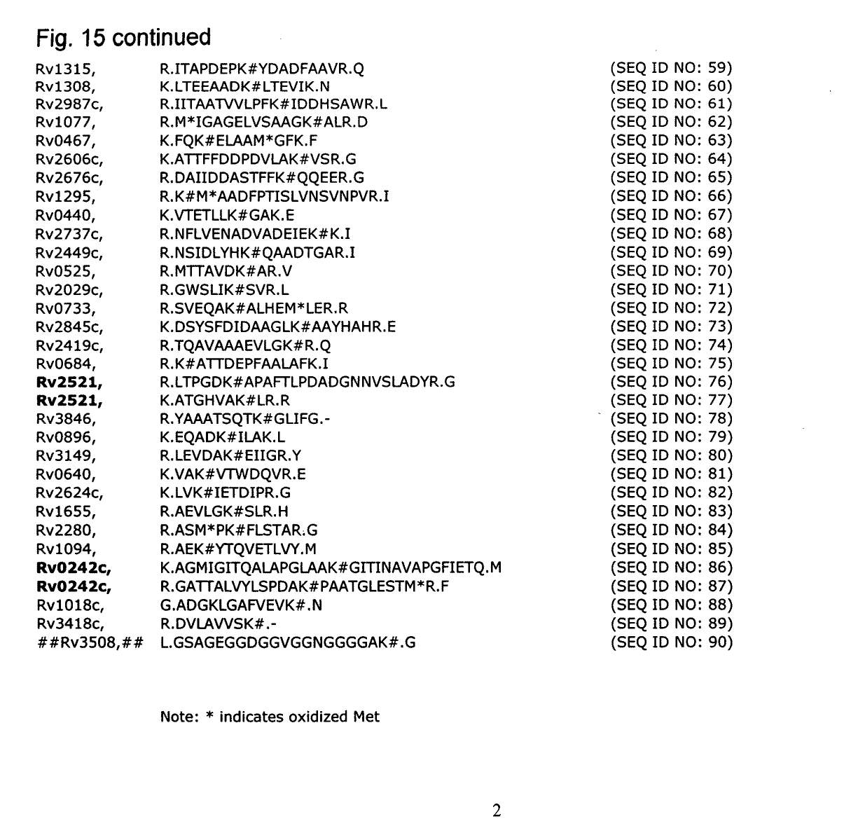

Monoclonal antibodies specific for Pup have also been generated as follows: about 1 mg of soluble His6-Pup was isolated and sent to Covance (Denver, Pa.) as an antigen against which to generate a bank of hybridomas. Hybridoma supernatants were cross checked for reactivity against Pup (lacking a His-tag), to reduce chances of selecting for antibodies reactive with the His6 tag. M. tuberculosis H37Rv and isogenic pafA mutant bacterial cultures were prepared for screening purposes. Total cell lysates of the above were harvested and used for western analysis using hybridoma supernatants in slot blot trays. Forty-eight (48) supernatants were tested at a 1:100 dilution on wild type Mtb lysates. Six (6) of the supernatants tested produced a robust signal. Westerns were repeated for these 6 supernatants and further tested against the pafA mutant, as a negative control sample. Separate hybridization containers were used to minimize potential for cross contamination and supernatants were used at dilutions of 1:500 and 1:2,000. SuperSignal West Pico reagent (available from Thermo Scientific, Pierce Protein Research Products) was used for detection following the manufacturer's guidelines. Two (2) hybridomas, designated 87 and 92, gave signal at both dilutions. Hybridoma 92 produced an exceptionally strong signal at both dilutions. See, for example, FIG. 13. Notably, neither hybridoma supernatant detected protein in the pafA mutant, demonstrating that the antibodies are highly specific for Pup. Moreover, monoclonal antibodies 87 and 92 demonstrate much greater sensitivity as compared to the Pup polyclonal antibodies used, for example, to detect pupylated substrates on immunoblots of Mtb lysates. See FIG. 3.

Methods of Tuberculosis (TB) Therapy

Mycobacteria are aerobic and nonmotile bacteria that are characteristically acid-alcohol fast. The exception to this rule is the species Mycobacterium marinum which has been shown to be motile within macrophages. All Mycobacterium species share a characteristic cell wall, thicker than in many other bacteria, which is hydrophobic, waxy, and rich in mycolic acids/mycolates. The cell wall thus presents challenges with respect to delivery of therapeutics that are capable of permeating the cell well.

It is well recognized that Mycobacterial infections are notoriously difficult to treat. The organisms are resistant to therapeutics at least in part due to their cell wall, which is neither truly Gram negative nor positive, and unique to the family. Moreover, Mycobacteria are naturally resistant to a number of antibiotics, such as penicillin, that destroy cell walls. The presence of the hydrophobic, waxy cell wall also enables Mycobacteria to survive prolonged exposure to acids, alkalis, detergents, oxidative bursts, lysis by complement and antibiotics, which naturally leads to antibiotic resistance. Most Mycobacteria are, however, susceptible to the antibiotics clarithromycin and rifamycin, but antibiotic-resistant strains are known to exist. The therapeutic agents identified using the methods of the present invention are envisioned as useful for both treatment of antibiotic-sensitive strains and antibiotic-resistant strains. Moreover, therapeutic agents identified using the methods of the present invention may be used in conjunction with known antibiotics, including clarithromycin and rifamycin. As described by Barrow et al. (Antimicrobial Agents and Chemotherapy 42:2682-2689, 1998), the entire contents of which is incorporated herein in its entirety, microsphere technology has been used to develop formulations of rifamycin to achieve targeted delivery to macrophages. A skilled practitioner would, therefore, appreciate that similar technology may be applied with respect to administration of agents identified using the methods of the present invention.

With respect to the complications associated with delivery, one of the objectives involved with the development of candidate therapeutics is the generation of small Pup mimetics or other small molecules that inhibit enzymes in the pupylation pathway. Transport of such small mimetics and molecules through the cell wall is more readily achieved than larger molecules.

Preparation of Pup-Encoding Nucleic Acid Molecules and Pup

Nucleic Acid Molecules: Nucleic acid molecules encoding Pup may be prepared by two general methods: (1) Synthesis from appropriate nucleotide triphosphates; or (2) Isolation from biological sources. Both methods utilize protocols well known in the art.

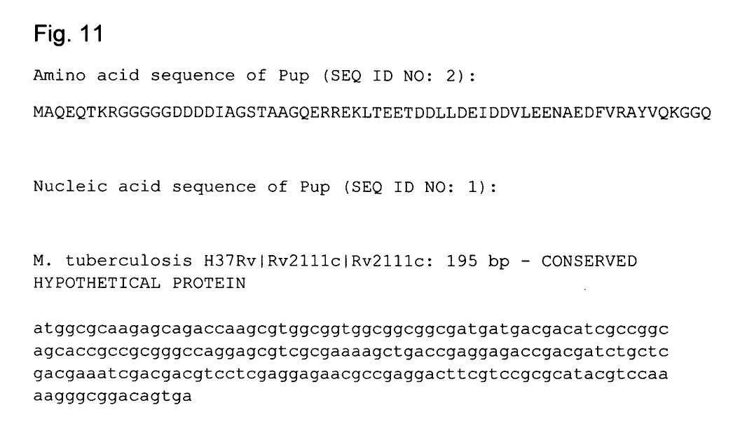

The availability of nucleotide sequence information, such as a full length DNA of SEQ ID NO: 1 (See FIG. 11), enables preparation of an isolated nucleic acid molecule of the invention by oligonucleotide synthesis. Synthetic oligonucleotides may be prepared by the phosphoramidite method employed in the Applied Biosystems 380A DNA Synthesizer or similar devices. The resultant construct may be purified according to methods known in the art, such as high performance liquid chromatography (HPLC). Double-stranded polynucleotides, such as a DNA molecule of the present invention, may be synthesized in stages, due to the size limitations inherent in current oligonucleotide synthetic methods. Synthetic DNA molecule constructed by such means may then be cloned and amplified in an appropriate vector. Nucleic acid sequences encoding Pup may be isolated from appropriate biological sources using methods known in the art. In that most bacterial genomes of interest have been completely sequenced, or will be in the near future, regular PCR off genomic DNA is typically used to isolate full length sequences. In an alternative embodiment, utilizing the sequence information provided by the full length DNA sequence, genomic clones encoding Pup may be isolated. Alternatively, full length DNA or genomic clones having homology to Pup may be isolated from other species, using oligonucleotide probes corresponding to predetermined sequences within the Pup gene. Genomic clones may isolated in accordance with the present invention may include regulatory elements such as, for example, promoter and enhancer elements.

In accordance with the present invention, nucleic acids having the appropriate level of sequence homology with the protein coding region of SEQ ID NO: 1 may be identified by using hybridization and washing conditions of appropriate stringency. For example, hybridizations may be performed using a hybridization solution comprising: 5×SSC, 5×Denhardt's reagent, 0.5-1.0% SDS, 100 micrograms/ml denatured, fragmented salmon sperm DNA, 0.05% sodium pyrophosphate and up to 50% formamide. Hybridization is generally performed at 37-42° C. for at least six hours. Following hybridization, filters are washed as follows: (1) 5 minutes at room temperature in 2×SSC and 0.5-1% SDS; (2) 15 minutes at room temperature in 2×SSC and 0.1% SDS; (3) 30 minutes-1 hour at 37° C. in 1×SSC and 1% SDS; (4) 2 hours at 42-65° C. in 1×SSC and 1% SDS, changing the solution every 30 minutes.

One common formula for calculating the stringency conditions required to achieve hybridization between nucleic acid molecules of a specified sequence homology is (Sambrook et al., 1989):

Tₘ=81.5° C. 16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5 VC with every 1% decrease in homology. Thus, targets with greater than about 75% sequence identity would be observed using a hybridization temperature of 42° C. Such a sequence would be considered substantially homologous to the nucleic acid sequence of the present invention.

As can be seen from the above, the stringency of the hybridization and wash depend primarily on the salt concentration and temperature of the solutions. In general, to maximize the rate of annealing of the two nucleic acid molecules, the hybridization is usually carried out at 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 micrograms/ml denatured salmon sperm DNA at 42° C. and wash in 2×SSC and 0.5% SDS at 55° C. for 15 minutes. A high stringency hybridization is defined as hybridization in 6×SSC, 5×Denhardt's solution, 0.5% SDS and 100 micrograms/ml denatured salmon sperm DNA at 42° C. and wash in 1×SSC and 0.5% SDS at 65° C. for 15 minutes. A very high stringency hybridization is defined as hybridization in 6×SSC, 5×Denhardt's solution, 0.5% SDS and 100 micrograms/ml denatured salmon sperm DNA at 42° C. and wash in 0.1×SSC and 0.5% SDS at 65° C. for 15 minutes.

Bioinformatics tools like BLAST searches of bacterial genomes (fully or partially sequenced) can also be used to identify homologues with potentially greater sensitivity than that achieved using hybridization based procedures.

Nucleic acids of the present invention may be maintained as DNA in any convenient cloning vector. In a preferred embodiment, clones are maintained in a plasmid cloning/expression vector, such as pBluescript (Stratagene, La Jolla, Calif.), which is propagated in a suitable E. coli host cell.

Pup-encoding nucleic acid molecules of the invention include DNA, genomic DNA, RNA, and fragments thereof which may be single- or double-stranded. Thus, this invention provides oligonucleotides (sense or antisense strands of DNA or RNA) having sequences capable of hybridizing with at least one sequence of a nucleic acid molecule of the present invention, such as selected segments of a DNA of SEQ ID NO: 1. Such oligonucleotides are useful as probes for detecting or isolating genes related to Pup.

It will be appreciated by persons skilled in the art that variants (e.g., allelic variants) of these sequences exist in bacterial populations and/or species, and must be taken into account when designing and/or utilizing oligonucleotides of the invention. Accordingly, it is within the scope of the present invention to encompass such variants, with respect to the Pup sequences disclosed herein or the oligonucleotides targeted to specific locations on the respective genes or RNA transcripts. With respect to the inclusion of such variants, the term “natural allelic variants” is used herein to refer to various specific nucleotide sequences and variants thereof that would occur in a given DNA population. Genetic polymorphisms giving rise to conservative or neutral amino acid substitutions in the encoded protein are examples of such variants. Additionally, the term “substantially complementary” refers to oligonucleotide sequences that may not be perfectly matched to a target sequence, but the mismatches do not materially affect the ability of the oligonucleotide to hybridize with its target sequence under the conditions described.

Thus, the coding sequence may be that shown in, for example, SEQ ID NO: 1, or it may be a mutant, variant, derivative or allele of either of these sequences. The sequence may differ from that shown by a change which is one or more of addition, insertion, deletion and substitution of one or more nucleotides of the sequence shown. Changes to a nucleotide sequence may result in an amino acid change at the protein level, or not, as determined by the genetic code.

Thus, nucleic acid according to the present invention may include a sequence different from the sequence shown in SEQ ID NO: 1, but which encodes a polypeptide with the same amino acid sequence.

On the other hand, the encoded polypeptide may comprise an amino acid sequence which differs by one or more amino acid residues from the amino acid sequence shown in SEQ ID NO: 2. See FIG. 11. Nucleic acid encoding a polypeptide which is an amino acid sequence mutant, variant, derivative or allele of the sequence shown in SEQ ID NO: 2 is further provided by the present invention. Nucleic acid encoding such a polypeptide may show greater than 60% identity with the coding sequence shown in SEQ ID NO: 1, greater than about 70% identity, greater than about 80% identity, greater than about 90% identity or greater than about 95% identity.

The present invention provides a method of obtaining a nucleic acid of interest, the method including hybridization of a probe having part or all of the sequence shown in SEQ ID NO: 1, or a complementary sequence thereto, to target nucleic acid. Successful hybridization leads to isolation of nucleic acid which has hybridized to the probe, which may involve one or more steps of polymerase chain reaction (PCR) amplification.

Such oligonucleotide probes or primers, as well as the full-length sequence (and mutants, alleles, variants, and derivatives) are useful in screening a test sample containing nucleic acid for the presence of alleles, mutants or variants of Pup, the probes hybridizing with a target sequence from a sample obtained from a cell, tissue, or organism being tested. The conditions of the hybridization can be controlled to minimize non-specific binding. Preferably stringent to moderately stringent hybridization conditions are used. The skilled person is readily able to design such probes, label them and devise suitable conditions for hybridization reactions, assisted by textbooks such as Sambrook et al (1989) and Ausubel et al (1992).

In some preferred embodiments, oligonucleotides according to the present invention that are fragments of the sequences shown in SEQ ID NO: 1, or any allele associated with endoribonuclease activity, are at least about 10 nucleotides in length, more preferably at least 15 nucleotides in length, more preferably at least about 20 nucleotides in length. Such fragments themselves individually represent aspects of the present invention. Fragments and other oligonucleotides may be used as primers or probes as discussed but may also be generated (e.g. by PCR) in methods concerned with determining the presence in a test sample of a sequence encoding a homolog or ortholog of Pup.

Polypeptides: Pup is the first prokaryotic ubiquitin-like protein to be identified. Indeed, Pup is the first known post-translational protein modifier to be identified in bacteria.

As described herein for the first time, Pup specifically conjugates to proteasome substrates in the pathogen Mycobacterium tuberculosis (Mtb). The identification of Pup facilitates the identification of prokaryotic proteasome substrates, which can be evaluated as suitable targets for therapeutics. The identification of Pup also provides for the establishment of screening assays directed to the identification of enzymes involved in the conjugation of Pup to substrates and enzymes required for de-pupylating (removal of conjugated Pup) for substrates. Moreover, methods and agents useful for modulating Pup activity and prokaryotic proteasome activity may be used to advantage as targeted therapeutics for the treatment of diseases associated with bacterial infections. More particularly, it is envisioned that agents identified using the method of the present invention may be useful for the treatment of patients with, for example, tuberculosis and leprosy.

A full-length Pup of the present invention may be prepared in a variety of ways, according to known methods. The protein may be purified from appropriate sources. This is not, however, a preferred method due to the low amount of protein likely to be present in a given cell type at any time. The availability of nucleic acid molecules encoding Pup enables production of this protein using in vitro expression methods known in the art. For example, a DNA or gene may be cloned into an appropriate in vitro transcription vector, such as pSP64 or pSP65 for in vitro transcription, followed by cell-free translation in a suitable cell-free translation system, such as wheat germ or rabbit reticulocyte lysates. In vitro transcription and translation systems are commercially available, e.g., from Promega Biotech, Madison, Wis. or BRL, Rockville, Md.

As indicated herein below, however, Pup expresses well in E. coli using pET24(b)+ (Novagen).

Alternatively, according to a preferred embodiment, larger quantities of Pup may be produced by expression in a suitable prokaryotic or eukaryotic system. For example, part or all of a DNA molecule, such as the sequence of SEQ ID NO: 1, may be inserted into a plasmid vector adapted for expression in a bacterial cell, such as E. coli. Such vectors comprise regulatory elements necessary for expression of the DNA in a host cell (e.g. E. coli) positioned in such a manner as to permit expression of the DNA in the host cell. Such regulatory elements required for expression include promoter sequences, transcription initiation sequences and, optionally, enhancer sequences.

Pup produced by gene expression in a recombinant prokaryotic or eukaryotic system may be purified according to methods known in the art. In a preferred embodiment, a commercially available expression/secretion system can be used, whereby the recombinant protein is expressed and thereafter secreted from the host cell, to be easily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope or the hemagglutinin epitope. Such methods are commonly used by skilled practitioners.

Pup of the invention, prepared by the aforementioned methods, may be analyzed according to standard procedures. For example, such proteins may be subjected to amino acid sequence analysis, according to known methods.

Polypeptides which are amino acid sequence variants, alleles, derivatives or mutants are also provided by the present invention. A polypeptide which is a variant, allele, derivative, or mutant may have an amino acid sequence that differs from that given in SEQ ID NO: 2 by one or more of addition, substitution, deletion and insertion of one or more amino acids. Preferred such polypeptides have Pup function, that is to say have one or more of the following properties: the ability to be conjugated to proteasome substrates, whereby conjugation serves to target the substrate for proteasome-mediated degradation; immunological cross-reactivity with an antibody reactive with the polypeptide for which the sequence is given in SEQ ID NO: 2; sharing an epitope with the polypeptide for which the sequence is given in SEQ ID NO: 2 (as determined for example by immunological cross-reactivity between the two polypeptides.

A polypeptide which is an amino acid sequence variant, allele, derivative or mutant of the amino acid sequence shown in SEQ ID NO: 2 may comprise an amino acid sequence which shares greater than about 35% sequence identity with the sequence shown, greater than about 40%, greater than about 50%, greater than about 60%, greater than about 70%, greater than about 80%, greater than about 90% or greater than about 95%. Particular amino acid sequence variants may differ from that shown in SEQ ID NO: 2 by insertion, addition, substitution or deletion of 1 amino acid, 2, 3, 4, 5-10, 10-20, 20-30, 30-40, 40-50, 50-100, 100-150, or more than 150 amino acids. For amino acid “homology”, this may be understood to be identity or similarity (according to the established principles of amino acid similarity, e.g., as determined using the algorithm GAP (Genetics Computer Group, Madison, Wis.). GAP uses the Needleman and Wunsch algorithm to align two complete sequences that maximizes the number of matches and minimizes the number of gaps. Generally, the default parameters are used, with a gap creation penalty=12 and gap extension penalty=4. Use of GAP may be preferred but other algorithms may be used including without limitation, BLAST (Altschul et al. (1990 J. Mol. Biol. 215:405-410); FASTA (Pearson and Lipman (1998) PNAS USA 85:2444-2448) or the Smith Waterman algorithm (Smith and Waterman (1981) J. Mol. Biol. 147:195-197) generally employing default parameters. Use of either of the terms “homology” and “homologous” herein does not imply any necessary evolutionary relationship between the compared sequences. The terms are used similarly to the phrase “homologous recombination”, i.e., the terms merely require that the two nucleotide sequences are sufficiently similar to recombine under appropriate conditions.

A polypeptide according to the present invention may be used in screening assays for molecules which affect or modulate Pup activity or function. Such molecules may be useful for research purposes.

Methods for Making Antibodies Immunologically Specific for Pup: The methods of the present invention encompass the use of antibodies or fragments thereof capable of specifically or selectively recognizing one or more Pup epitopes. Such antibodies may include, but are not limited to, polyclonal antibodies, monoclonal antibodies (mAbs), humanized or chimeric antibodies, single chain antibodies, Fab fragments, F(ab′)₂ fragments, Fv fragments, fragments produced by a Fab expression library, anti-idiotypic (anti-Id) antibodies, and epitope-binding fragments of any of the above.

Such antibodies may be used, for example, in the detection of Pup in a biological sample and may, therefore, be utilized as part of a diagnostic or prognostic technique whereby cells isolated from a patient may be tested to determine if bacteria are present. Suitable techniques are known in the art. As indicated herein for the first time, the ability of Pup to conjugate to proteasome substrates makes it possible to identify such substrates (using the attached Pup, e.g., as a tag for isolation) and the identification and characterization of proteasome substrates enables further screening assays to identify enzymes involved in pupylation and de-pupylation of these substrates. Antibodies immunologically specific for Pup are, therefore, useful for visualizing Pup-conjugated substrates and, by extension, are also useful tools for isolating pupylated substrates and complexes comprising pupylated substrates. Accordingly, such antibodies are included in a kit whereby polypeptides may be isolated by virtue of their conjugation to Pup. Such antibodies are also included as reagents in a kit for use in a diagnostic or prognostic technique. Such antibodies may also be utilized in conjunction with, for example, compound screening methods, such as those described herein below, for the evaluation of the effect of test compounds on Pup gene product levels and/or activity.

Described herein are methods for the production of antibodies or fragments thereof. Any of such antibodies or fragments thereof may be produced by standard immunological methods or by recombinant expression of nucleic acid molecules encoding the antibody or fragments thereof in an appropriate host organism.

For the production of antibodies against Pup, various host animals may be immunized by injection with Pup, or a portion thereof, or a fusion protein comprising part or all of Pup. Such host animals may include, but are not limited to rabbits, mice, and rats. Various adjuvants may be used to increase the immunological response, depending on the host species, including but not limited to Freund's (incomplete), mineral gels such as aluminum hydroxide, surface active substances such as lysolecithin, pluronic polyols, polyanions, peptides, oil emulsions, keyhole limpet hemocyanin (KLH), and dinitrophenol. The present inventors have found that, because Pup is so small, it is desirable to conjugate Pup to KLH so as to boost the immunological response to Pup.

Polyclonal antibodies are heterogeneous populations of antibody molecules derived from the sera of animals immunized with an antigen, such as Pup, or an antigenic functional derivative thereof. For the production of polyclonal antibodies, host animals such as those described above, may be immunized by injection with Pup or a fragment thereof supplemented with adjuvants as described above.

Monoclonal antibodies (mAbs), which are homogeneous populations of antibodies to a particular antigen or epitope thereof, may be obtained by any technique that provides for the production of antibody molecules by continuous cell lines in culture. These include, but are not limited to, the hybridoma technique of Kohler and Milstein, (1975, Nature 256:495; and U.S. Pat. No. 4,376,110), the human B-cell hybridoma technique (Kosbor et al., 1983, Immunology Today 4:72; Cole et al., 1983, Proc. Natl. Acad. Sci. USA 80:2026), and the EBV-hybridoma technique (Cole et al., 1985, Monoclonal Antibodies And Cancer Therapy, Alan R. Liss, Inc., pp. 77). Such antibodies may be of any immunoglobulin class including IgG, IgM, IgE, IgA, IgD and any subclass thereof. A hybridoma producing a mAb of this invention may be cultivated in vitro or in vivo. Production of high titers of mAbs in vivo renders this method a particularly preferred method of production of Pup antibodies.

Techniques developed for the production of “chimeric antibodies” (Morrison et al., 1984, Proc. Natl. Acad. Sci., 81, 6851-6855; Neuberger et al., 1984, Nature 312, 604-608; Takeda et al., 1985, Nature 314, 452-454), whereby the genes from a mouse antibody molecule of appropriate antigen specificity are spliced to genes from a human antibody molecule of appropriate biological activity, are also encompassed by the present invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 5,816,397). The invention thus contemplates chimeric antibodies that are specific or selective for Pup.

Examples of techniques that have been developed for the production of humanized antibodies are known in the art. See, e.g., Queen, U.S. Pat. No. 5,585,089 and Winter, U.S. Pat. No. 5,225,539. An immunoglobulin light or heavy chain variable region consists of a “framework” region interrupted by three hypervariable regions, referred to as complementarity-determining regions (CDRs). The extent of the framework region and CDRs have been precisely defined. See, “Sequences of Proteins of Immunological Interest”, Kabat, E. et al., U.S. Department of Health and Human Services (1983). Briefly, humanized antibodies are antibody molecules from non-human species having one or more CDRs from the non-human species and framework regions from a human immunoglobulin molecule. The invention includes the use of humanized antibodies that are specific or selective for Pup in the methods of the invention.

The methods of the invention encompass the use of an antibody or derivative thereof comprising a heavy or light chain variable domain, said variable domain comprising (a) a set of three complementarity-determining regions (CDRs), in which said set of CDRs are from a monoclonal antibody to a gene product encoded by a Pup nucleic acid sequence (e.g., SEQ ID NO: 1), and (b) a set of four framework regions, in which said set of framework regions differs from the set of framework regions in the Pup monoclonal antibody, and in which said antibody or derivative thereof immunospecifically binds to the gene product encoded by a Pup gene sequence. Preferably, the set of framework regions is from a human monoclonal antibody, e.g., a human monoclonal antibody that does not bind the gene product encoded by the Pup gene sequence.

Phage display technology can be used to increase the affinity of an antibody to Pup. This technique is useful for obtaining high affinity antibodies to Pup that could be used for the diagnosis and/or prognosis of a subject with, for example, tuberculosis. The technology, referred to as affinity maturation, employs mutagenesis or CDR walking and re-selection using Pup antigen to identify antibodies that bind with higher affinity to the antigen when compared with the initial or parental antibody (see, e.g., Glaser et al., 1992, J. Immunology 149:3903). Mutagenizing entire codons rather than single nucleotides results in a semi-randomized repertoire of amino acid mutations. Libraries can be constructed consisting of a pool of variant clones each of which differs by a single amino acid alteration in a single CDR and which contain variants representing each possible amino acid substitution for each CDR residue. Mutants with increased binding affinity for the antigen can be screened by contact with the immobilized mutants containing labeled antigen. Any screening method known in the art can be used to identify mutant antibodies with increased avidity to the antigen (e.g., ELISA) (See Wu et al., 1998, Proc Natl. Acad. Sci. USA 95:6037; Yelton et al., 1995, J. Immunology 155:1994). CDR walking which randomizes the light chain is also possible (See Schier et al., 1996, J. Mol. Bio. 263:551).

Alternatively, techniques described for the production of single chain antibodies (U.S. Pat. No. 4,946,778; Bird, 1988, Science 242:423; Huston et al., 1988, Proc. Natl. Acad. Sci. USA 85:5879; and Ward et al., 1989, Nature 334:544) can be adapted to produce single chain antibodies against Pup. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., 1988, Science 242:1038).

The methods of the invention include using an antibody to Pup, peptide or other derivative, or analog thereof that is a bispecific antibody (see generally, e.g., Fanger and Drakeman, 1995, Drug News and Perspectives 8:133-137). Such a bispecific antibody is genetically engineered to recognize both (1) an epitope and (2) one of a variety of “trigger” molecules, e.g., Fc receptors on myeloid cells, and CD3 and CD2 on T-cells, that have been identified as being able to cause a cytotoxic T-cell to destroy a particular target. Such bispecific antibodies can be prepared either by chemical conjugation, hybridoma, or recombinant molecular biology techniques known to the skilled artisan.

Antibody fragments that recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science 246:1275-1281) to allow rapid and easy identification of monoclonal Fab fragments with the desired specificity.

Antibodies, for example, or fragments of antibodies, such as those described herein, useful in the present invention may be used to quantitatively or qualitatively detect the presence of Pup or naturally occurring variants or peptide fragments thereof. The antibodies (or fragments thereof) useful in the present invention may, additionally, be employed histologically, as in immunofluorescence or immunoelectron microscopy, for in situ detection of Pup gene products or conserved variants or peptide fragments thereof. In situ detection may be accomplished by removing a histological specimen from a subject, such as paraffin embedded sections of tissue, e.g., lung tissues and applying thereto a labeled antibody of the present invention. The antibody (or fragment) is preferably applied by overlaying the labeled antibody (or fragment) onto a biological sample. Since the Pup gene product is expressed as an intracellular protein, it is desirable to introduce the antibody inside the cell, for example, by making the cell membrane permeable. Alternatively, or in addition, Pup may be released from the bacteria or even the cellular host wherein the bacteria resides, thereby creating a pool of Pup that antibodies can recognize and neutralize extra-bacterially or extracellularly.

Through the use of such a procedure, it is possible to determine not only the presence of a Pup gene product, or naturally occurring variants thereof or peptide fragments, but also its distribution in the examined tissue. Using the methods of the present invention, those of ordinary skill will readily perceive that any of a wide variety of histological methods (such as staining procedures) can be modified in order to achieve such in situ detection.

Immunoassays for Pup or conserved variants or peptide fragments thereof will typically comprise contacting a sample, such as a biological fluid, tissue or a tissue extract, freshly harvested cells, or lysates of cells which have been incubated in cell culture, in the presence of an antibody that specifically or selectively binds to a Pup gene product, e.g., a detectably labeled antibody capable of identifying Pup or conserved variants or peptide fragments thereof, and detecting the bound antibody by any of a number of techniques well-known in the art (e.g., Western blot, ELISA, FACS).

The biological sample may be brought in contact with and immobilized onto a solid phase support or carrier such as nitrocellulose, or other solid support that is capable of immobilizing cells, cell particles or soluble proteins. The support may then be washed with suitable buffers followed by treatment with the detectably labeled antibody that selectively or specifically binds to Pup. The solid phase support may then be washed with the buffer a second time to remove unbound antibody. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The anti-Pup antibody can be detectably labeled by linking the same to an enzyme and using the labeled antibody in an enzyme immunoassay (EIA) (Voller, A., “The Enzyme Linked Immunosorbent Assay (ELISA)”, 1978, Diagnostic Horizons 2:1, Microbiological Associates Quarterly Publication, Walkersville, Md.); Voller, A. et al., 1978, J. Clin. Pathol. 31: 507-520; Butler, J. E., 1981, Meth. Enzymol. 73:482; Maggio, E. (ed.), 1980, Enzyme Immunoassay, CRC Press, Boca Raton, Fla.; Ishikawa, E. et al., (eds.), 1981, Enzyme Immunoassay, Kgaku Shoin, Tokyo). The enzyme that is bound to the antibody reacts with an appropriate substrate, preferably a chromogenic substrate, in such a manner as to produce a chemical moiety detectable, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect Pup encoded polypeptides through the use of a radioimmunoassay (RIA). See, for example, Weintraub, B., Principles of Radioimmunoassays, Seventh Training Course on Radioligand Assay Techniques, The Endocrine Society, March, 1986. The radioactive isotope can be detected by such means as the use of a gamma counter or a scintillation counter or by autoradiography.

An antibody of the invention can also be labeled with a fluorescent compound. When the fluorescently labeled antibody is exposed to light of the proper wavelength, its presence can be detected due to fluorescence emission. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody can also be detectably labeled by coupling it to a chemiluminescent compound. The presence of the chemiluminescent-tagged antibody is determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

In various embodiments, the present invention provides methods for the measurement of Pup, and the uses of such measurements in clinical applications using antibodies immunologically specific for Pup.

Uses of Pup-Encoding Nucleic Acids Pup Polypeptides and Antibodies Thereto

As indicated herein, Pup nucleic acids, proteins and antibodies thereto, according to this invention, may be used as research tools to identify prokaryotic proteasome targets. Proteasomal targets so identified may prove to be suitable targets for therapeutics for use in the treatment of patients (e.g., humans) afflicted with a bacterial disease.

Accordingly, Pup nucleic acids, proteins and antibodies thereto may be used in a variety of methods described herein, such as a method for identifying proteasome substrates (Pup substrates); a method for identifying modulators of Pup activity (in a bacterial cell or in a test tube); a method for identifying an enzyme that covalently attaches Pup to a proteasome substrate in a bacterial cell (which may be referred to herein as activation and conjugation enzymes of Pup); a method for identifying de-pupylating enzymes.

Pup nucleic acids (and vectors comprising same), proteins and antibodies thereto may also be used in compositions.

Pup nucleic acids (and vectors comprising same), proteins and antibodies thereto may also be components in kits. An exemplary kit is described herein for expressing and purifying a fusion protein in a eukaryotic cell.

Moreover, the synthesis of Pup-traps, for example, is expected to lead to the development of therapeutics for the treatment of diseases caused by bacterial infections.

As indicated herein, the synthesis of Pup-traps involves the production of recombinant Pup in E. coli, which is chemically modified at the C-terminus with a moiety like vinyl sulfone or vinylmethyl ester.

Pup-Encoding Nucleic Acids: Pup-encoding nucleic acids may be used for a variety of purposes in accordance with the present invention. Pup-encoding DNA, RNA, or fragments thereof may be used as probes to detect the presence of and/or expression of genes encoding Pup-like proteins. Methods in which Pup-encoding nucleic acids may be utilized as probes for such assays include, but are not limited to: (1) in situ hybridization; (2) Southern hybridization (3) northern hybridization; and (4) assorted amplification reactions such as PCR.

Pup-encoding nucleic acids of the invention may also be utilized as probes to identify related genes from other bacterial, plant, or animal species. As is well known in the art, hybridization stringencies may be adjusted to allow hybridization of nucleic acid probes with complementary sequences of varying degrees of homology. Thus Pup-encoding nucleic acids may be used to advantage to identify and characterize other genes of varying degrees of relation to Pup, thereby enabling further characterization of the prokaryotic proteasomal pathway. Additionally, they may be used to identify genes encoding proteins that interact with Pup (e.g., by the “interaction trap” technique), which should further accelerate identification of the components involved in pathways involved in the prokaryotic proteasomal pathway, including molecules involved in the conjugation of Pup to substrates and removal thereof.

Nucleic acid molecules, or fragments thereof, encoding Pup may also be utilized to control the production of Pup, thereby regulating the amount of protein available to participate in pupylation. Alterations in the physiological amount of Pup protein may dramatically affect the activity of other protein factors involved in protein degradation.

Pup and Antibodies Thereto: As described herein, purified Pup proteins, or fragments thereof, produced via expression of Pup encoding nucleic acids of the present invention may be used to produce polyclonal or monoclonal antibodies which also may serve as sensitive detection reagents for the presence and accumulation of Pup (or complexes containing Pup) in human cells and potentially cells of related mammals and primates. Recombinant techniques enable expression of fusion proteins containing part or all of Pup. The full length protein or fragments of the protein may be used to advantage to generate an array of monoclonal antibodies specific for various epitopes of the protein, thereby providing a potentially even greater sensitivity for detection of the protein in cells.

Polyclonal or monoclonal antibodies immunologically specific for Pup may be used in a variety of assays designed to detect and quantitate the protein. Such assays include, but are not limited to: (1) flow cytometric analysis; (2) immunochemical localization of Pup in cells; and (3) immunoblot analysis (e.g., dot blot, Western blot) of extracts from various cells. Additionally, as described above, anti-Pup antibodies, for example, can be used for purification of Pup and orthologs thereof (e.g., affinity column purification and immunoprecipitation). Anti-Pup antibodies may also be used in affinity purification experiments designed to isolate pupylated polypeptides and complexes thereof.

Methods for visualizing Pup

Methods for generating antibodies immunologically specific for Pup are described in detail herein. Such methods include those used for generating monoclonal and polyclonal Pup antibodies. As described in detail herein, Pup antibodies can be generated in response to full length Pup, or any antigenic fragment thereof (e.g., an antigenic peptide), or a fusion protein comprising all or an antigenic fragment of Pup.

Anti-Pup antibodies may be labeled directly to facilitate detection of antibodies bound to Pup in a cell. Alternatively, secondary antibodies which recognize an epitope on the primary antibody (i.e., the anti-Pup antibody) may be labeled to enable detection of Pup-antibody complexes. Methods wherein labeled secondary or tertiary antibodies are used to visualize the binding of a primary antibody to its specific target are a matter of routine practice. Indeed, labeling of secondary or tertiary antibodies is generally considered to amplify the signal and, thereby, promote the level of detection to enable visualization of rare antibody-antigen complexes. Various labels that are useful for such purposes are known in the art and described herein.

Visualization of immunofluorescent complexes comprising Pup and associated antibodies may be visualized using any means available for detection of fluorescently labeled molecules at a level of resolution sufficient for detecting pupylated substrates and complexes comprising pupylated substrates. Such means include fluorescent microscopes equipped with high magnification lenses, and confocal microscopes. Skilled practitioners are familiar with the technical parameters required for detection at this level of resolution and are knowledgeable with regard to the instrumentation required for such determinations.

From the foregoing discussion, it can be seen that Pup-encoding nucleic acids, Pup expressing vectors, and anti-Pup antibodies of the invention can be used to produce large quantities of Pup protein, detect Pup gene expression and alter Pup accumulation for purposes of assessing the genetic and protein interactions involved in prokaryotic proteasome substrate degradation and virulence associated with certain prokaryotes.

General Methods for Identifying Compounds Capable of Modulating Pup Activity

A structure of the Pup can be determined by standard means familiar in the art. Generally, such means begin with polypeptide modeling that utilizes a selected protein structure derived by conventional means, e.g., X-ray crystallography, NMR, homology modeling, or the like. Such methods are known to those skilled in the art.

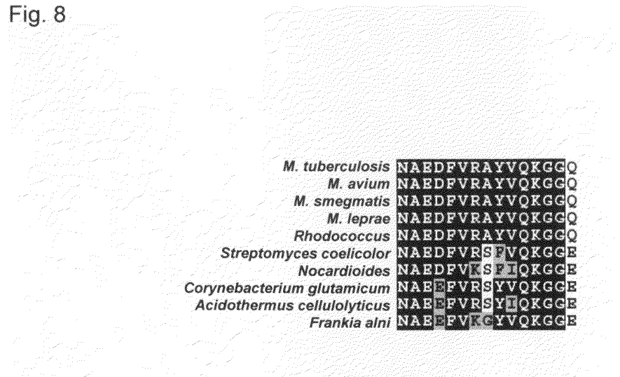

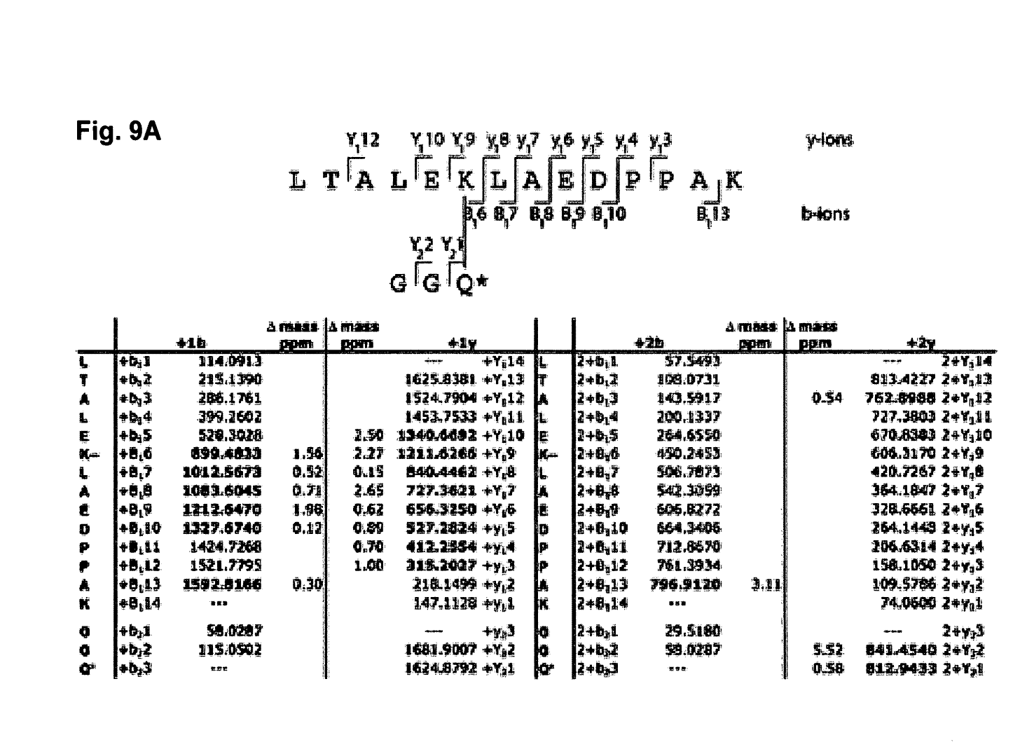

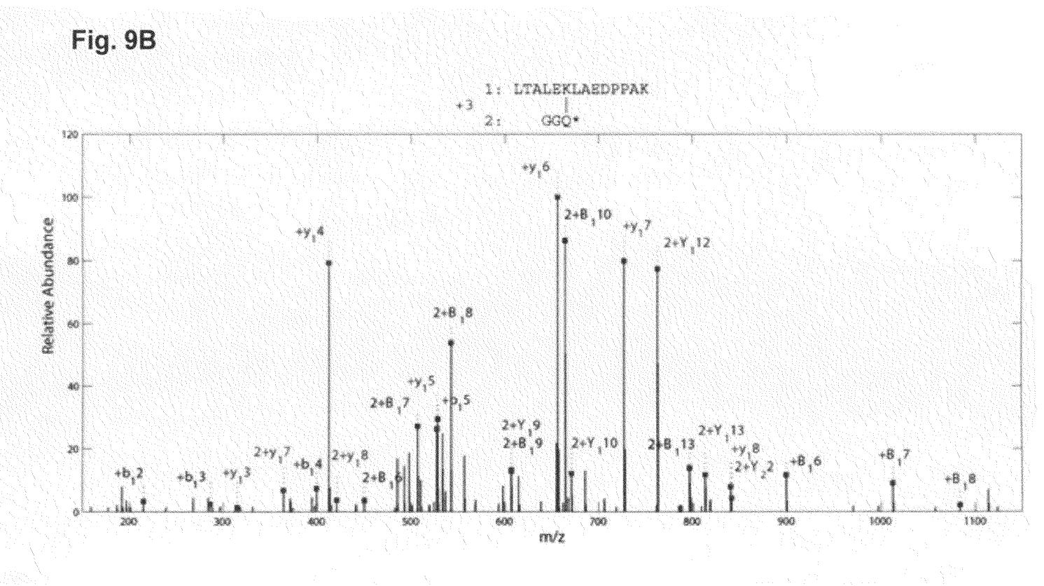

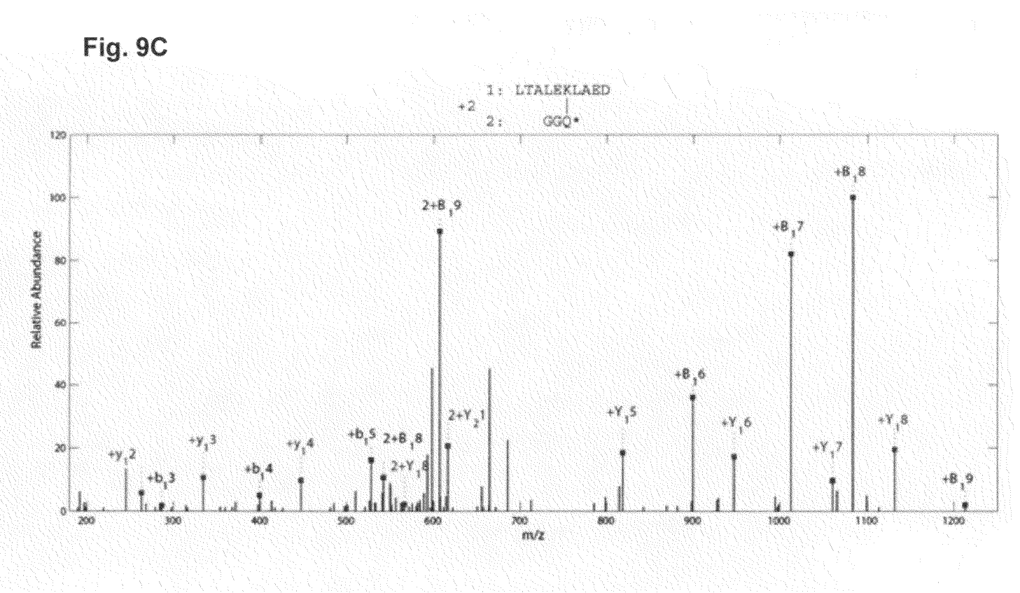

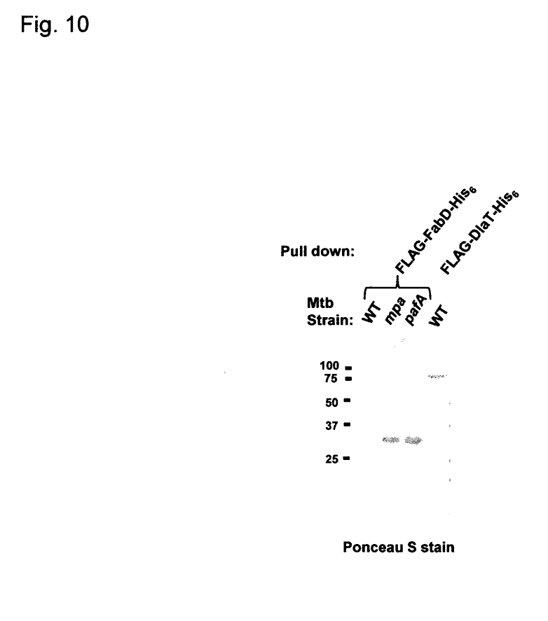

Based on information presented herein, suitable peptide targets in Pup include, but are not limited to, those residues and regions listed below. Suitable peptide targets in Pup include the C-terminus as shown in FIG. 8. The C-terminus is significant because it conjugates to substrates and interacts with Mpa (the ATPase) as demonstrated by two hybrid screens performed by the present inventors. Also included are critical residues and small peptides encompassing these critical residues (e.g. 5-10 residue peptides comprising these residues and flanking residues thereof) which are identified on the basis of high resolution information determined for Pup. Along these lines, the highly conserved penultimate di-glycine is maintained in all ubiquitin-like proteins characterized to date. Moreover, the present inventors have determined that if either di-glycine is mutated or moved, Pup is no longer active.

In one embodiment of the invention, a crystal structure of Pup is used as a target in a virtual ligand screening procedure that seeks to identify, via computer docking methods, candidate compounds from a vast compound library which bind with high affinity to the target site.

In another embodiment, structural information pertaining to Pup is used to design compounds predicted to bind to Pup and interfaces formed between molecules (e.g., polypeptides, nucleic acid sequences), and such compounds are tested for high affinity binding.

In specific embodiments, candidate compounds and “designed compounds” are selected which modulate binding of Pup, for example, to enzymes involved in Pup conjugation to substrates or removal therefrom. Such compounds may either enhance or inhibit binding of Pup to enzymes involved in conjugation or removal of Pup from substrates. Such compounds may, in turn, effectuate an increase or a decrease in Pup-mediated substrate degradation by the proteasome. Compounds derived or obtained from such an approach scoring the highest in the docking procedure can also be tested in cell-free assays, as well as cell-based assays (which are both described in detail herein below), to determine their efficacy in modulating Pup activity.

Any compounds which show efficacy in biological assays may then be co-crystallized with Pup to identify the binding site(s). In a further embodiment of the invention, candidate compounds able to bind Pup are modified by methods known in the art to further improve specific characteristics, e.g., to increase efficacy and/or specificity and/or solubility. Selected compounds exhibiting the most desired characteristics are designated lead compounds, and further tested in, for example, animal models of tuberculosis (such as, e.g., mice) to measure their efficacy.

Virtual Ligand Screening Via Flexible Docking Technology

Current docking and screening methodologies can select small sets of likely lead candidate ligands from large libraries of compounds using a specific protein structure. Such methods are described, for example, in Abagyan and Totrov (2001) Current Opinion Chemical Biology 5:375-382, herein specifically incorporated by reference in its entirety.

Virtual ligand screening (VLS) based on high-throughput flexible docking is useful for designing and identifying compounds able to bind to a specific protein structure. VLS can be used to virtually sample a large number of chemical molecules without synthesizing and experimentally testing each one. Generally, the methods start with polypeptide modeling which uses a selected protein structure derived by conventional means, e.g., X-ray crystallography, NMR, homology modeling. A set of compounds and/or molecular fragments are then docked into the selected binding site using any one of the existing docking programs, such as for example, MCDOCK (Liu et al. (1999) J. Comput. Aided Mol. Des. 13:435-451), SEED (Majeux et al. (1999) Proteins 37:88-105; DARWIN (Taylor et al. (2000) Proteins 41:173-191; MM (David et al. (2001) J. Comput. Aided Mol. Des. 15:157-171. Compounds are scored as ligands, and a list of candidate compounds predicted to possess the highest binding affinities generated for further in vitro and in vivo testing and/or chemical modification.

In one approach of VLS, molecules are “built” into a selected binding pocket prior to chemical generation. A large number of programs are designed to “grow” ligands atom-by-atom [see, for example, GENSTAR (Pearlman et al. L (1993) J. Comput. Chem. 14:1184), LEGEND (Nishibata et al. (1993) J. Med. Chem. 36:2921-2928), MCDNLG (Rotstein et al. (1993) J. Comput-Aided Mol. Des. 7:23-43), CONCEPTS (Gehlhaar et al. (1995) J. Med. Chem. 38:466-472] or fragment-by-fragment [see, for example, GROUPBUILD (Rotsein et al. (1993) J. Med. Chem. 36:1700-1710), SPROUT (Gillet et al. (1993) J. Comput. Aided Mol. Des. 7:127-153), LUDI (Bohm (1992) J. Comput. Aided Mol. Des. 6:61-78), BUILDER (Roe (1995) J. Comput. Aided Mol. Des. 9:269-282), and SMOG (DeWitte et al. (1996) J. Am. Chem. Soc. 118:11733-11744].

Methods for scoring ligands for a particular protein are known which allow discrimination between the small number of molecules able to bind the protein structure and the large number of non-binders. See, for example, Agagyan et al. (2001) supra, for a report on the growing number of ligands successfully identified via virtual ligand docking and screening methodologies.

For example, Nishibata et al. (1993) J. Med. Chem. 36:2921-2928, describe the ability of a structure construction program to generate inhibitory molecules based on the three-dimension structure of the active site of a molecule, dihydrofolate reductase. The program was able to predict molecules having a similar structure to four known inhibitors of the enzyme, providing strong support that new lead compounds can be obtained with knowledge of the target three-dimensional structure. Similarly, Gillet et al. (1993) J. Computer Aided Mol. Design 7:127-153 describe structure generation through artificial intelligence techniques based on steric constraints (SPROUT).

Agents Identified by the Screening Methods of the Invention

The invention provides methods for identifying agents (e.g., candidate compounds or test compounds) that bind with high affinity to Pup. Agents identified by the screening method of the invention are useful as candidate anti-tuberculosis therapeutics.

Examples of agents, candidate compounds or test compounds include, but are not limited to, nucleic acids (e.g., DNA and RNA), carbohydrates, lipids, proteins, peptides, peptidomimetics, small molecules and other drugs. Exemplary nucleic acids determined to be capable of modulating Pup activity include, but are not limited to: Pup siRNA molecules described herein. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233, each of which is incorporated herein in its entirety by reference.

Libraries of compounds may be presented, e.g., presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (19900 Science 249:386-390; Devlin (1990) Science 249:404-406; Cwirla et al. (1990) Proc. Natl. Acad. Sci. USA 87:6378-6382; and Felici (1991) J. Mol. Biol. 222:301-310), each of which is incorporated herein in its entirety by reference.

Screening Assays

Small molecules identified through the above described virtual ligand docking and screening methodologies are further tested in in vitro and in vivo assays. In one embodiment, agents that interact with (i.e., bind to) Pup are identified in a cell-based assay system.

In accordance with this embodiment, cells expressing Pup or a functional fragment thereof, are contacted with a candidate compound or a control compound and the ability of the candidate compound to interact with Pup is determined. If desired, this assay may be used to screen a plurality (e.g. a library) of candidate compounds. The cell, for example, can be of prokaryotic origin (e.g., E. coli) or eukaryotic origin (e.g., yeast or mammalian). Exemplary cells and cell lines that can be used in cell-based assays include, but are not limited to, bone marrow derived macrophages from mice, monocytic cell lines such as murine J774, and human Mono Mac 6 cell lines. Depending on the goal of the cell-based assay performed, a skilled artisan would know to select cells that can be infected by the Pup-containing bacteria in question.

With respect to bacteria, intact bacteria may be screened for sensitivity to acidified nitrite (which makes nitric oxide). This is a proven screening assay since this is the phenotype used to identify mpa and pafA. Potential hits could then be tested in a macrophage assay using, for example, bone marrow derived macrophages isolated from mice. The compound could be evaluated based on its ability to reduce the number of Mtb colony forming units (CFU) in Mtb infected macrophages, as compared to a control compound. Such an approach would also yield useful information with respect to the toxicity of the compound under evaluation to mammalian cells.

Further, the cells can express Pup or a fragment thereof endogenously or be genetically engineered to express Pup or a Pup fragment. In certain instances, Pup or a Pup fragment is labeled, for example with a radioactive label (such as ³²P, ³⁵S or ¹²⁵I) or a fluorescent label (such as fluorescein isothiocyanate, rhodamine, phycoerythrin, phycocyanin, allophycocyanin, o-phthaldehyde or fluorescamine) to enable detection of an interaction between Pup and a candidate compound. The ability of the candidate compound to bind to Pup can be determined by methods known to those of skill in the art. For example, the interaction between a candidate compound and Pup can be determined by flow cytometry, a scintillation assay, immunoprecipitation or western blot analysis.

In another embodiment, agents that interact with (i.e., bind to) Pup, or a functional fragment thereof, are identified in a cell-free assay system. In accordance with this embodiment, a native or recombinant Pup or fragment thereof is contacted with a candidate compound or a control compound and the ability of the candidate compound to interact with Pup is determined. If desired, this assay may be used to screen a plurality (e.g. a library) of candidate compounds. In one embodiment, Pup or a fragment thereof is first immobilized, by, for example, contacting with an immobilized antibody which specifically recognizes and binds to Pup, or by contacting a purified preparation of Pup or fragment thereof, with a surface designed to bind proteins. Pup or a fragment thereof may be partially or completely purified (e.g., partially or completely free of other polypeptides) or part of a cell lysate. Further, Pup or a fragment thereof may be part of a fusion protein comprising Pup or a biologically active portion thereof, and a domain such as glutathionine-S-transferase. Alternatively, Pup or a fragment thereof can be biotinylated using techniques well known to those of skill in the art (e.g., biotinylation kit, ThermoScientific; Rockford, Ill.). The ability of the candidate compound to interact with Pup can be determined by detection methods known to those of skill in the art.

In another embodiment, agents that modulate the Pup activity are identified or tested in an animal model. Examples of suitable animals include, but are not limited to, mice, rats, rabbits, monkeys, guinea pigs, dogs and cats. Preferably, the animal used represents a model of a disease caused by a bacterial infection (e.g., tuberculosis). In accordance with this embodiment, the test compound or a control compound is administered (e.g., orally, rectally or parenterally such as intraperitoneally or intravenously) to a suitable animal and the effect on the level of activity is determined.

A skilled practitioner would appreciate that Pup RNAi (siRNA) molecules are exemplary Pup modulatory agents. Such RNAi molecules may be used to advantage to significantly reduce and/or eliminate Pup expression levels in cells into which the molecules have been introduced. The potential of this technology will become fully realized when a bacterial equivalent of Dicer is identified.

The basic molecular biology techniques used to practice the methods of the invention are well known in the art, and are described for example in Sambrook et al., 1989, Molecular Cloning: A Laboratory Manual, Cold Spring Harbor Laboratory, New York; Ausubel et al., 1988, Current Protocols in Molecular Biology, John Wiley & Sons, New York; and Ausubel et al., 2002, Short Protocols in Molecular Biology, John Wiley & Sons, New York).

Therapeutic Uses of Agents Able to Bind and/or Modulate Pup Activity and Pupylation

The invention provides for treatment of a disease caused by a bacterial infection (e.g., tuberculosis) by administration of a therapeutic compound identified using the above-described methods. Such compounds include, but are not limited to proteins, peptides, protein or peptide derivatives or analogs, antibodies, nucleic acids, and small molecules.

The invention provides methods for treating patients afflicted with a bacterial infection (e.g., tuberculosis) comprising administering to a subject an effective amount of a compound identified by the method of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In a specific embodiment, a non-human mammal is the subject.

Formulations and methods of administration that can be employed when the compound comprises a nucleic acid are described above; additional appropriate formulations and routes of administration are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu (1987) J. Biol. Chem. 262:4429-4432), and construction of a nucleic acid as part of a retroviral or other vector. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally, e.g., by local infusion during surgery, topical application, e.g., by injection, by means of a catheter, or by means of an implant, said implant being of a porous, non-porous, or gelatinous material, including membranes, such as silastic membranes, or fibers.

In another embodiment, the compound can be delivered in a vesicle, in particular a liposome (see Langer (1990) Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton (1987) CRC Crit. Ref. Biomed. Eng. 14:201; Buchwald et al. (1980) Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, i.e., a target tissue or tumor, thus requiring only a fraction of the systemic dose (see, e.g., Goodson, in Medical Applications of Controlled Release, supra, vol. 2, pp. 115-138 (1984)). Other controlled release systems are discussed in the review by Langer (1990, Science 249:1527-1533).

Pharmaceutical Compositions

The present invention also provides pharmaceutical compositions. Such compositions comprise a therapeutically effective amount of an agent, and a pharmaceutically acceptable carrier. In a particular embodiment, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin, incorporated in its entirety by reference herein. Such compositions will contain a therapeutically effective amount of the compound, preferably in purified form, together with a suitable amount of carrier so as to provide the form for proper administration to the subject. The formulation should suit the mode of administration.

In a particular embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment of a disease caused by a bacterial infection (e.g., tuberculosis) can be determined by standard clinical techniques based on the present description. In addition, in vitro assays may optionally be employed to help identify optimal dosage ranges. The precise dose to be employed in the formulation will also depend on the route of administration, and the seriousness of the disease, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Suppositories generally contain active ingredient in the range of 0.5% to 10% by weight; oral formulations preferably contain 10% to 95% active ingredient. Effective doses may be extrapolated from dose-response curves derived from in vitro or animal model test systems.

Nucleic Acids

The invention provides methods of identifying agents capable of binding and/or modulating Pup. Accordingly, the invention encompasses administration of a nucleic acid encoding a peptide or protein capable of modulating an activity of Pup, as well as antisense sequences or catalytic RNAs capable of interfering with the expression and/or activity of Pup.

In one embodiment, a nucleic acid comprising a sequence encoding a peptide or protein capable of competitively binding to Pup is administered. Any suitable methods for administering a nucleic acid sequence available in the art can be used according to the present invention.

Methods for administering and expressing a nucleic acid sequence are generally known in the area of gene therapy. For general reviews of the methods of gene therapy, see Goldspiel et al. (1993) Clinical Pharmacy 12:488-505; Wu and Wu (1991) Biotherapy 3:87-95; Tolstoshev (1993) Ann. Rev. Pharmacol. Toxicol. 32:573-596; Mulligan (1993) Science 260:926-932; and Morgan and Anderson (1993) Ann. Rev. Biochem. 62:191-217; May (1993) TIBTECH 11(5): 155-215. Methods commonly known in the art of recombinant DNA technology which can be used in the present invention are described in Ausubel et al. (eds.), 1993, Current Protocols in Molecular Biology, John Wiley & Sons, NY; and Kriegler (1990) Gene Transfer and Expression, A Laboratory Manual, Stockton Press, NY.

In a particular aspect, the compound comprises a nucleic acid encoding a peptide or protein capable of binding to and/or modulating an activity of Pup (such as the ability to be conjugated to a proteasome substrate or be recognized by a enzyme involved in pupylation or de-pupylation of a substrate), such nucleic acid being part of an expression vector that expresses the peptide or protein in a suitable host. In particular, such a nucleic acid has a promoter operably linked to the coding region, said promoter being inducible or constitutive (and, optionally, tissue-specific). In another particular embodiment, a nucleic acid molecule is used in which the coding sequences and any other desired sequences are flanked by regions that promote homologous recombination at a desired site in the genome, thus providing for intrachromosomal expression of the nucleic acid (Koller and Smithies (1989) Proc. Natl. Acad. Sci. USA 86:8932-8935; Zijlstra et al. (1989) Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject, known as “ex vivo gene therapy”.

In another embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors.

In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al. (1989) Nature 342:435-438).

In a further embodiment, a retroviral vector can be used (see Miller et al. (1993) Meth. Enzymol. 217:581-599). These retroviral vectors have been modified to delete retroviral sequences that are not necessary for packaging of the viral genome and integration into host cell DNA. The nucleic acid encoding a desired polypeptide to be used in gene therapy is cloned into the vector, which facilitates delivery of the gene into a subject. More detail about retroviral vectors can be found in Boesen et al. (1994) Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al. (1994) J. Clin. Invest. 93:644-651; Kiem et al. (1994) Blood 83:1467-1473; Salmons and Gunzberg (1993) Human Gene Therapy 4:129-141; and Grossman and Wilson (1993) Curr. Opin. in Genetics and Devel. 3:110-114.

Adenoviruses may also be used effectively in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson (1993) Current Opinion in Genetics and Development 3:499-503 present a review of adenovirus-based gene therapy. Bout et al. (1994) Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al. (1991) Science 252:431-434; Rosenfeld et al. (1992) Cell 68:143-155; Mastrangeli et al. (1993) J. Clin. Invest. 91:225-234; PCT Publication WO94/12649; and Wang, et al. (1995) Gene Therapy 2:775-783. Adeno-associated virus (AAV) has also been proposed for use in gene therapy (Walsh et al. (1993) Proc. Soc. Exp. Biol. Med. 204:289-300; U.S. Pat. No. 5,436,146).

Another suitable approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr (1993) Meth. Enzymol. 217:599-618; Cohen et al. (1993) Meth. Enzymol. 217:618-644; Cline (1985) Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a preferred embodiment, epithelial cells are injected, e.g., subcutaneously. In another embodiment, recombinant skin cells may be applied as a skin graft onto the subject; recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver. In a preferred embodiment, the cell used for gene therapy is autologous to the subject that is treated.

In another embodiment, the nucleic acid to be introduced for purposes of gene therapy may comprise an inducible promoter operably linked to the coding region, such that expression of the nucleic acid is controllable by adjusting the concentration of an appropriate inducer of transcription.

Direct injection of a DNA coding for a peptide or protein capable of binding to and/or modulating an activity of Pup may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection.

Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

With respect to kits, small ubiquitin-related modifier (SUMO) has been used to develop a protein expression and purification system wherein eukaryotic proteins are expressed and affinity purified from bacterial lysates (Invitrogen) by virtue of a hexahistidine tag and processed with a protease that de-sumoylates the fusion protein, leaving the purified protein of interest. Because eukaryotes make SUMO or highly related molecules, the use of Pup would likely be more favorable if it was required that protein expression is performed in a eukaryotic cell (e.g., yeast or a cell line). Indeed, SUMO has the potential to bind to endogenous proteins, including the protease that cleaves it.

It is, therefore, envisioned that an expression kit comprising a Pup fusion protein vector may be useful for synthesis of recombinant proteins that must be expressed in a eukaryotic expression system. Accordingly, such Pup fusion protein vectors may encode an epitope tag for affinity purification, such as a histidine tag. Alternatively, the prokaryotic (bacterial) Pup protein portion of the expressed fusion protein may be used to advantage as a tag which facilitates affinity purification of recombinant proteins that are expressed in a eukaryotic cell. As indicated above, expression and purification in eukaryotic cells is desired, if not essential, under circumstances wherein protein modification systems (e.g. glycosylation and myristoylation) are required for expression of fully functional proteins. The protease used to remove Pup from a recombinant protein is not likely to be found in eukaryotic cells, thus enabling high recovery of Pup-fusion proteins from a eukaryotic expression system. The Pup fusion protein vector of the present invention may further comprise a linker that operably links the polypeptide sequence to the Pup sequence and such a linker may comprise a specific cleavage site incorporated therein to facilitate release of the polypeptide of interest from the Pup sequence.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and described the methods and/or materials in connection with which the publications are cited.

### Example I

### Methods and Materials

Bacterial strains and culture conditions. Bacterial strains used in this study are listed in Table 1. E. coli strains used for cloning and expression were grown in LB Miller broth (Difco) at 37° C. with aeration on an orbital shaker or on LB agar. E. coli strains were chemically transformed as previously described (Sambrook et al., Molecular Cloning: A Laboratory Manual (Cold Spring Harbor Laboratory Press, Cold Spring Harbor, 1989)). Minimal medium using E-salts (Maloy et al., Genetic Analysis of Pathogenic Bacteria (Cold Spring Harbor Laboratory Press, Cold Spring Harbor, 1996), pp. 603) was supplemented with 1% lactose, maltose or glucose as needed. Mtb strains were grown in Middlebrook 7H9 broth (Difco) supplemented with 0.2% glycerol, 0.05% Tween-80, 0.5% bovine serum albumin, 0.2% dextrose and 0.085% sodium chloride. Mtb cultures were grown without shaking in 75 cm² vented flasks (Corning) in humidified incubators with 5% CO₂ at 37° C. 7H11 agar (Difco) supplemented with oleic acid, albumin, dextrose and catalase (BBL) was used for growth on solid medium. Msm strains were grown at 37° C. with shaking in 7H9 broth supplemented with 0.2% glycerol and 0.05% Tween-80 or on 7H11 agar with 0.2% glycerol. Mycobacteria were transformed as described elsewhere (Hatfull et al., Molecular Genetics of Mycobacteria (ASM Press, Washington, D.C., 2000).

The final concentrations of antibiotics used for E. coli were: ampicillin, 200 μg/ml; hygromycin, 150 μg/ml; and kanamycin, 100 μg/ml. For Mtb and Msm, hygromycin and kanamycin were each used at 50 μg/ml. Trimethoprim (Trim) was used at 50 μg/ml for Msm.

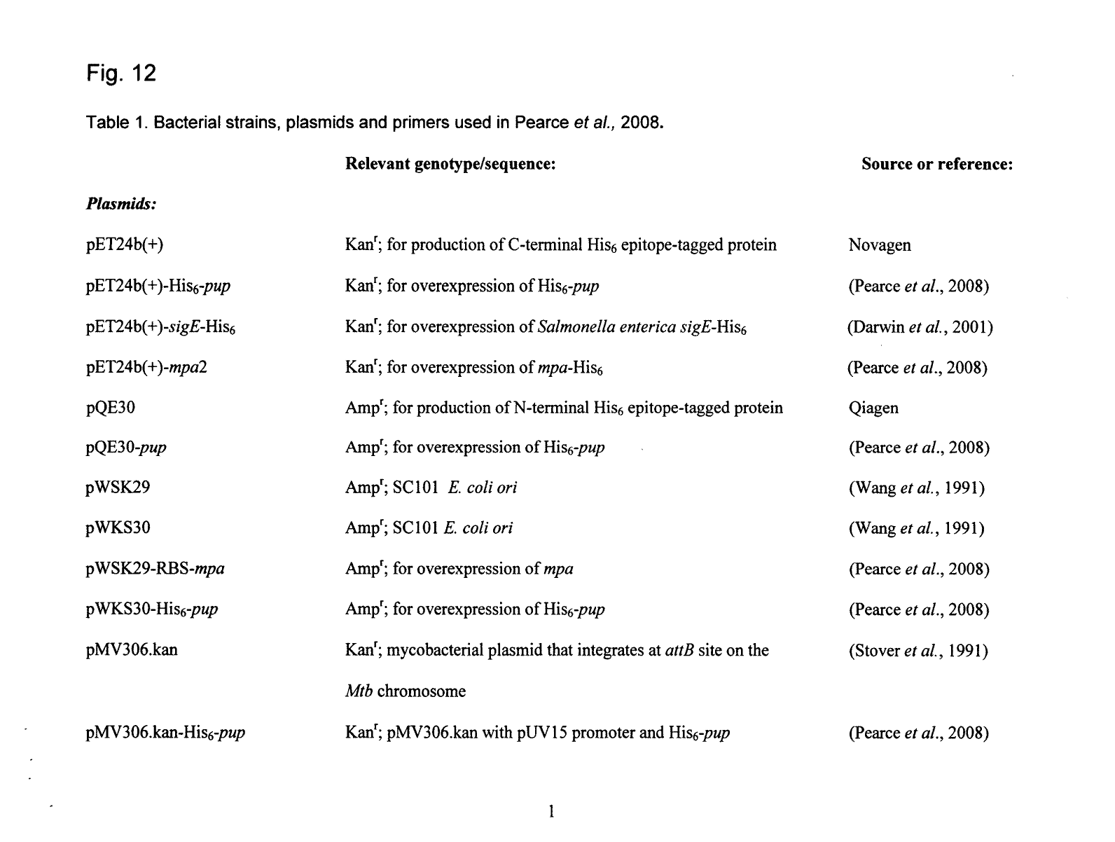

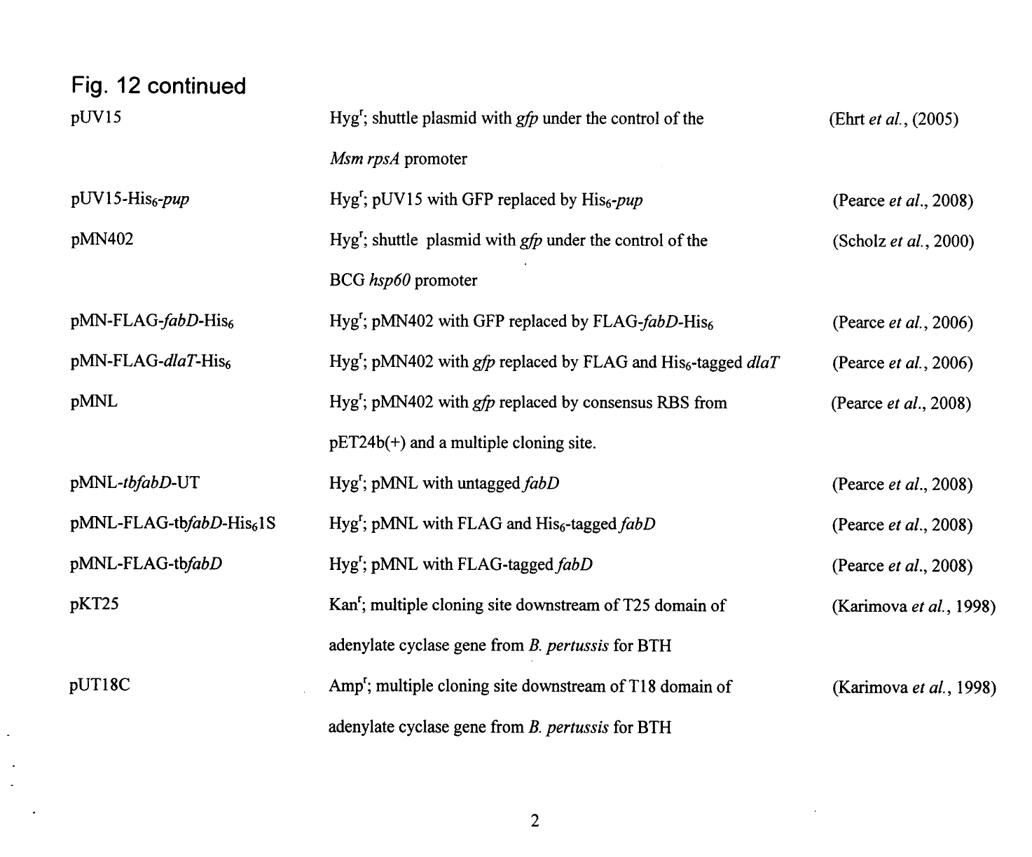

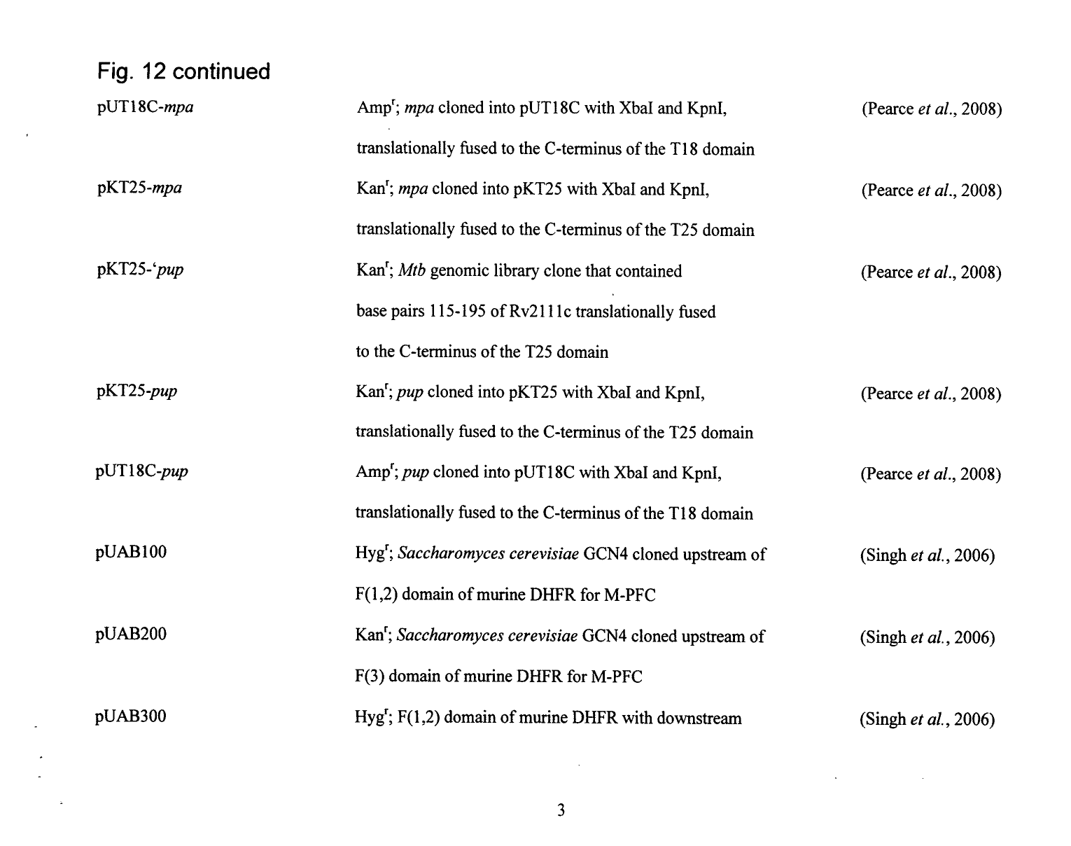

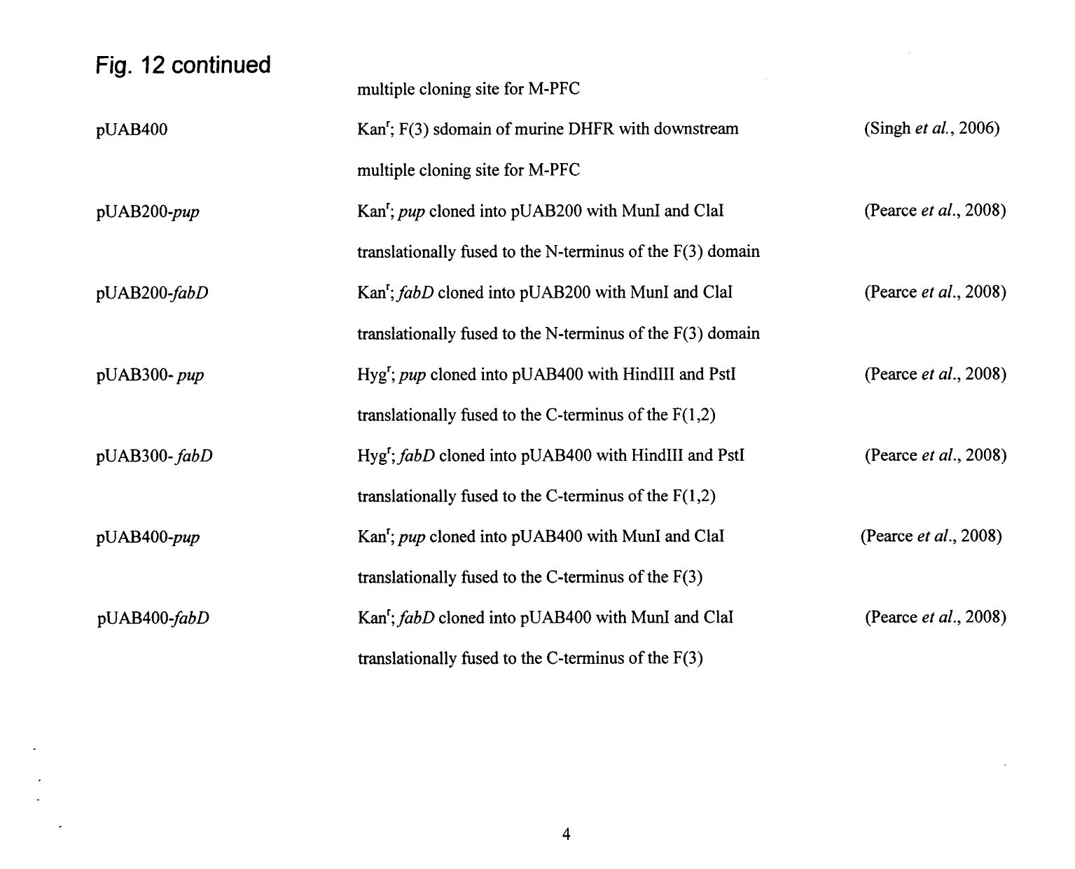

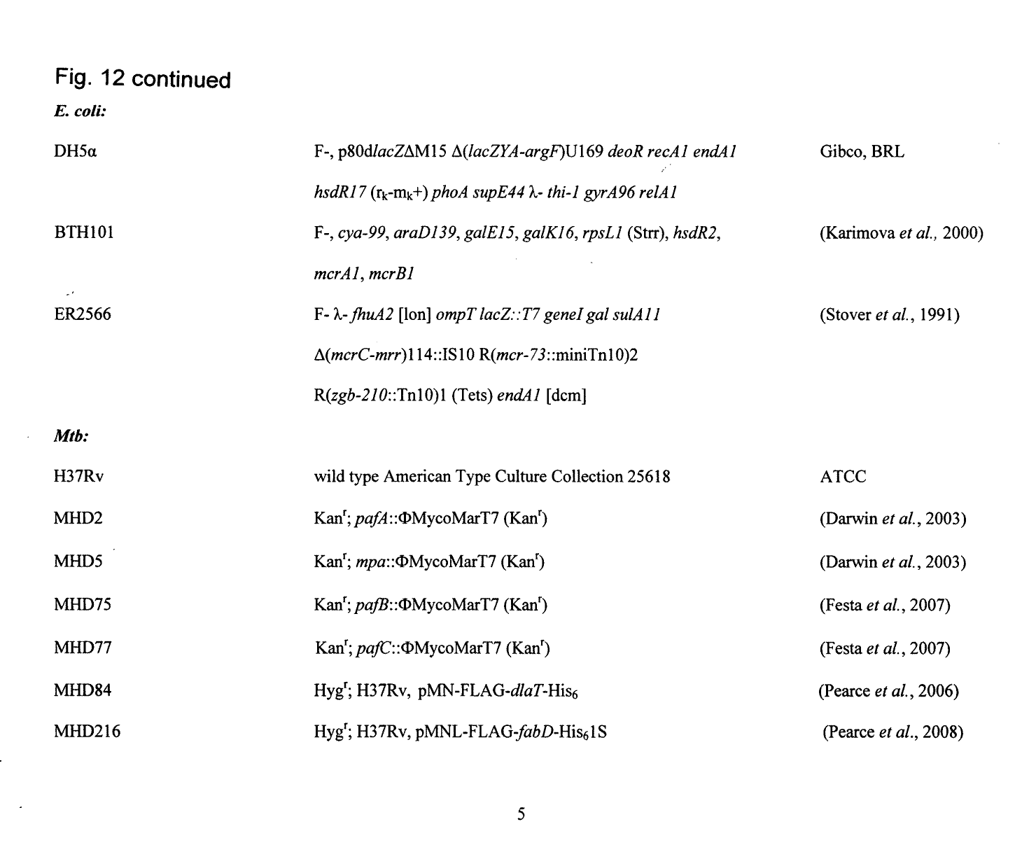

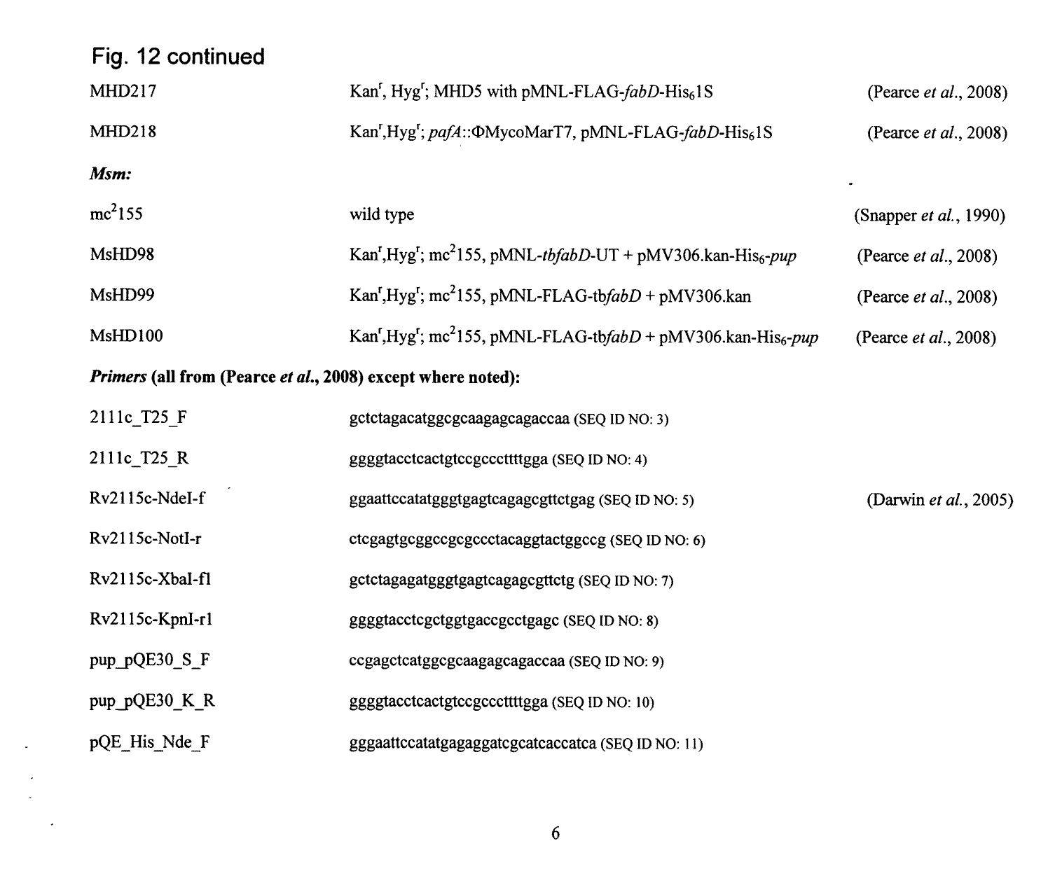

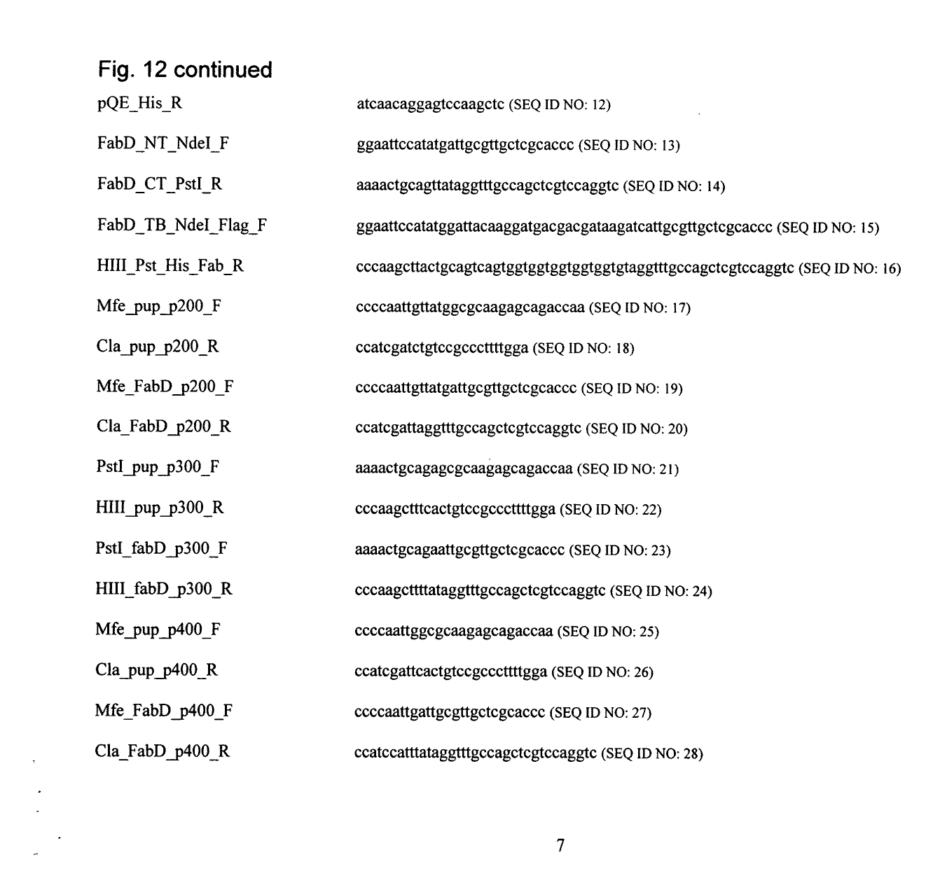

Antibodies, plasmids and primers. Plasmids and primers used in experiments described in Example I are listed in FIG. 12 (Table 1).

For overexpression of native mpa in E. coli, PCR products were cloned into the NdeI and NotI sites of plasmid pET24b(+), maintaining the stop codon. For over expression from a plasmid with a different origin of replication, pET24b(+)-mpa was digested with XbaI and NotI and the fragment, including the consensus ribosome binding site (RBS) from pET24b(+), was sub-cloned into pWSK29.

pET24b(+) was used to express His₆-pup in E. coli for purification for in vitro interaction studies and for antibody production. pup was first cloned into the SacI and KpnI sites in pQE30 (which adds a His₆ tag to the 5′ end of pup) via restriction sites included in the primers used to PCR amplify pup. Using pQE30-pup as a template, a second set of primers was used to amplify His₆-pup and clone it into the NdeI and HindIII sites of pET24b(+). This strategy included the nucleotides AGAGGATCG (RGS epitope; SEQ ID NO: 100) before the His₆ sequence and the nucleotides GGATCCGCATGCGAG; SEQ ID NO: 101) between His₆ and pup.

The previously reported pMN-FLAG-fabD-His₆ (Pearce et al., EMBO J. 25, 5423 (2006)) was used as a template for making additional fabD constructs. fabD was PCR amplified using Fab_NT_NdeI_F and Fab_CT_PstI_R, and cloned into the NdeI and PstI restriction sites of pMNL, which has the Mtb hsp60 promoter for expression of the cloned gene (creating pMNL-tbfabD-UT). We made a new FLAG/His₆ epitope-tagged fabD because we found that two forms of FabD were being synthesized: one that had both FLAG and His₆ epitopes and one that had only the His₆ epitope. This was likely due to the presence of two recognizable RBS in the transcript expressed from the original pMN-FLAG-fabD-His₆. To make an epitope-tagged fabD with only one start of translation, we used PCR using primers FabTBNdeIFlafF and HIII-PStI-HisFabR and cloned PCR products into the NdeI and the HindIII sites of pMNL, creating pMNL-FLAG-tbfabD-His₆1S. To make FabD with only the FLAG epitope tag, we subcloned the N-terminal fragment of pMNL-FLAG-tbfabD-His₆1S containing the FLAG epitope into the NdeI and PvuII restriction sites of pMNL-tbfabD-UT, resulting in pMNL-FLAG-tbfabD.

pMV306-kan-His₆-pup was made as follows: His₆-pup was sub-cloned from pET24b(+)-His₆-pup into the NdeI and HindIII restriction sites of the pUV15 mycobacterial expression vector creating pUV15-His₆-pup. The pUV15 vector contains the strong mycobacterial promoter from the Msm rpsA gene (Ehrt et al., Nucleic Acids Res 33, e21 (2005)). This plasmid was digested with SpeI and ClaI to subclone the UV15 promoter with His₆-pup into the XbaI and ClaI restriction sites of pMV306-kan, which integrates into the attB site on the chromosome. To make pWKS30-His₆-pup, His₆-pup was sub-cloned from pET24b(+)-His₆-pup into the XbaI and HindIII restriction sites of pWKS30 downstream of the T7 promoter.

Pfu Polymerase (Stratagene) was used for all PCR. All primers were purchased from Invitrogen. Restriction enzymes were purchased from New England Biolabs. All clones were sequenced by GENEWIZ, Inc. to confirm the correct DNA sequence.

For antibody production, His₆-Pup was purified from E. coli ER2566 with pET24b(+)His₆-Pup under native conditions as described in the QIAexpressionist manual (Qiagen). Polyclonal rabbit antibodies were produced by Covance. Antibodies to His₆-Pup were affinity purified as described elsewhere (Darwin et al., Mol Microbiol 55, 561 (2005)). FLAG antibodies were purchased from Sigma; antibodies to penta-His were purchased from Qiagen. Polyclonal antibodies to DlaT-His₆ were a gift from Ruslana Bryk and Carl Nathan and used as described elsewhere (Darwin et al., Mol Microbiol 55, 561 (2005); Lin et al., Mol. Microbiol. 59, 1405 (2006); Tian et al., Mol Microbiol 57, 859 (August, 2005)). Horseradish peroxidase (HRP) coupled anti-rabbit secondary antibodies were used according to manufacturer's instructions (GE Healthcare).

E. coli BTH screen. A genomic Mtb DNA library was constructed by cloning 300-800 bp DNA fragments from a limited Sau3AI digestion of Mtb chromosomal DNA into the BamHI site of pKT25. 100 ligations were used to transform DH5α. Plasmid DNA was purified from all 100 pools, each representing ˜1,000 clones. The BTH screen was performed using methods described elsewhere (Karimova et al., Proc Natl Acad Sci USA 95, 5752 (1998)). Briefly, mpa was cloned into in the KpnI and XbaI sites of pUT18C and used to transform BTH101. This bait strain was then transformed with 1 μl of each library pool, totaling 100 transformations. Bacteria were plated on minimal media containing 1% lactose as the carbon source. Plates were incubated at 30° C. for four days and then for an additional four days at 25° C. Growth indicated putative interactions between Mpa and another protein. Colonies were patched onto MacConkey indicator agar containing 1% maltose as the carbon source and incubated at 30° C. for two days and then for an additional two days at 25° C. Red colonies indicated a positive interaction. We recovered the pKT25 library plasmids from these clones and transformed BTH101 with the bait plasmid or empty vector to either confirm interactions or eliminate false positives, respectively. Plasmids that conferred growth on minimal media with 1% lactose were sequenced. Full-length pup was cloned into the XbaI and KpnI sites of pKT25 and pUT18C to confirm interaction with the Mpa bait.

Msm protein fragment complementation assay. M-PFC takes advantage of the fact that murine dihydrofolate reductase (mDHFR) has a 12,000-fold lower affinity for the antibiotic trimethoprim (Trim) than the bacterial DHFR (Singh et al., Proc Natl Acad Sci USA 103, 11346 (Jul. 25, 2006)). Like the E. coli BTH system, the interaction of two Mtb proteins fused to complimentary domains of mDHFR can restore mDHFR activity, allowing Msm to grow on selective (Trim-containing) media. Plasmids used in the assay are listed in Table 1. pUAB100 and pUAB200 containing the Saccharomyces cerevisiae GCN4 leucine zipper domain were used as a positive control. In some cases, we replaced GCN4 in pUAB200 with an Mtb gene. Pairs of plasmids were used to transform WT Msm and bacteria were grown on 7H11 agar for five days at 37° C. Single colonies were inoculated into 150 μl 7H9 broth and incubated for five days at 37° C. 10 μl of each culture was then inoculated onto 7H11 agar with or without Trim, and incubated at 37° C. for three days.

Affinity chromatography. For in vitro validation of the Pup/Mpa interaction, 1 L cultures of E. coli ER2566 containing either pET24b(+)-His₆-pup or pET24b(+)-sigE-His₆ were induced with 1 mM IPTG at an OD₆₀₀ of ˜0.5 for 5 h at 37° C. Cell lysates were made and proteins were purified at 4° C. exactly as described in the QIAexpressionist manual. Purified proteins were dialyzed overnight in 4 l of 50 mM NaH₂PO₄/300 mM NaCl then quantified using a NanoDrop spectrophotometer. For untagged Mpa, a 50 ml culture of E. coli strain ER2566 containing pWKS30-RBS-mpa was induced with 1 mM IPTG at an OD₆₀₀ of ˜0.5 for 5 h at 37° C. (“Mpa lysate”). A cell lysate (“empty cell lysate”) from an E. coli strain containing pET24b(+) was also prepared. To examine protein-protein interactions, 60 μg of purified His₆-tagged proteins or 900 μl of empty cell lysate were incubated with 25 μl of Ni-NTA agarose for 1.5 h at 4° C. with agitation. The agarose was collected by centrifugation and the supernatant was discarded. 50 μl of Mpa lysate in 450 μl of lysis buffer was incubated with the agarose for 1.5 h at 4° C. with agitation. The agarose was collected, the supernatant was saved (“flow through”), and the agarose was washed with 750 μl wash buffer (50 mM NaH₂PO₄/300 mM NaCl/30 mM imidazole) three times. The agarose was resuspended in 200 μl of elution buffer (50 mM NaH₂PO₄/300 mM NaCl/250 mM imidazole) and collected by centrifugation. The supernatant was saved (“elution”).

For analysis of the Pup˜FabD interaction in Msm, 50 ml cultures of WT Msm containing either pMNL-FLAG-fabD+pMV306-kan-His₆-pup, pMNL-fabDUT+pMV306-kan-His₆-pup or pMNL-FLAG-fabD+pMV306-kan were grown to an OD₅₈₀ of ˜1.0. Bacteria were collected by centrifugation and resuspended in 4 ml of FLAG buffer (50 mM Tris HCl, 150 mM NaCl, pH 7.4). 1 ml aliquots of resuspended cells were transferred to bead beating tubes each with 250 μl of zirconia silica beads (BioSpec Products). Cells were lysed by bead beating four times for 1 m each in a BioSpec Mini Bead Beater. Samples were clarified by centrifugation and were then passed through a 0.2μ filter. The soluble lysates were quantified using a NanoDrop spectrophotometer and 7.8 mg were incubated with 250 μl of ANTI-FLAG M2 Affinity Gel (Sigma) for 2 h at 4° C. with agitation. The matrix was washed two times 5 ml with FLAG buffer in a Poly-Prep column (Bio-Rad). Proteins were eluted with two 300 μl fractions using 100 μg/ml FLAG peptide (Sigma) and then concentrated using a Microcon YM-30 concentrator (Millipore).

To test for the Pup˜FabD interaction in E. coli, 50 ml cultures of WT E. coli containing either the combination pMNL-FLAG-fabD+pWKS30-His₆-pup, or pMNL-FLAG-fabD+pWKS30 were grown to an OD₅₈₀ of ˜1.0. Bacteria were processed and purified as described above except cells were lysed by bead beating three times, 1 m each.

To purify His₆-tagged proteins from Mtb, 30 ml cultures were grown to an OD₅₈₀ of ˜0.8-1.0. Bacteria were collected by centrifugation and resuspended in 3 ml of Ni-NTA lysis buffer. Cells were lysed and quantified as described for Msm and 4.65 mg were incubated with 40 μl of Ni-NTA agarose for 2 hours at 4° C. with agitation. The agarose was collected by centrifugation, the supernatant was saved (flow through), and the agarose was washed three times with 750 μl Ni-NTA wash buffer. The agarose was resuspended in 100 μl of Ni-NTA elution buffer and collected by centrifugation, and the supernatant was saved (elution). Samples were boiled for 10 min, and proteins were detected by immunoblotting.

MS analysis. To purify FLAG-FabD˜His-Pup for MS analysis, 2 L of WT Msm containing pMNL-FLAG-fabD+pMV306(kan)-His₆-pup were grown to an OD₅₈₀ of ˜1.0. Bacteria were collected by centrifugation and resuspended in 66 ml of lysis buffer (see above) and lysed by sonication. Cellular debris was removed by centrifugation, the soluble lysate was filtered using a 0.2μ syringe filter and the lysate was incubated with 1.6 ml of Ni-NTA agarose for 2 h at 4° C. with agitation. The agarose was collected in a polypropylene column and washed once with 10 ml and twice with 5 ml of Ni-NTA wash buffer. Proteins were eluted twice with 1 ml Ni-NTA elution buffer. 500 μl of ANTI-FLAG M2 Affinity Gel was added directly to the elution and incubated for 2 h at 4° C. with agitation. The matrix was washed twice with 5 ml with FLAG buffer in a Poly-Prep column. Two 350 μl and one 250 μl elutions were collected using 100 μg/ml FLAG peptide, combined and concentrated using a Microcon YM-30 concentrator. The 45 kD band was excised from a 12% SDS-PAGE gel stained with CBB, de-stained and in-gel digested with trypsin. The resulting peptide mixture was subjected to LC-MS/MS in an LTQ-OrbitrapXL hybrid mass spectrometer (ThermoFisher, San Jose, Calif.). The instrument was operated in data-dependent mode using a setup similar to one described previously (Haas et al., Mol Cell Proteomics 5, 1326 (July, 2006)). Here, the six most abundant ions detected in the survey MS scan were selected for MS/MS in the Orbitrap. The resolution was set to 1.5×10⁴ and AGC to 2×10⁴ for the MS/MS scans. MS/MS spectra were assigned by searching them with the SEQUEST algorithm (Eng et al., J. Am. Soc. Mass. Spectrom. 5, 976 (1994)) against the M. tuberculosis sequence database.

Immunoblotting. Total protein lysates were prepared from equivalent cell numbers. We harvested 11 OD₅₈₀ equivalent cell numbers by centrifugation and washed in 5 ml of 0.05% Tween-80 in phosphate buffered saline, resuspended in 500 μl of lysis buffer (100 mM Tris-Cl, 100 mM KCl, 1 mM EDTA, 5 mM MgCl₂, pH 8) and transferred to bead beating tubes with 250 μl of zirconia silica beads. Cells were lysed by bead beating three times for 1 min each time. Lysates were clarified by centrifugation and 270 μl of soluble cell lysate was mixed with 90 μl of 4× protein sample buffer. The samples were boiled at 100° C. for 10 min and equal volumes representing equivalent cell numbers were separated by 12% SDS-PAGE. Proteins were transferred to nitrocellulose and blocked in either 5% milk or 1% BSA. For immunoblots, experimental membranes were stripped and incubated with anti-DlaT to check equivalent loading of samples. Detection of HRP was performed using either SuperSignal West Pico or West Femto Chemiluminescent Substrate (ThermoScientific).

Results

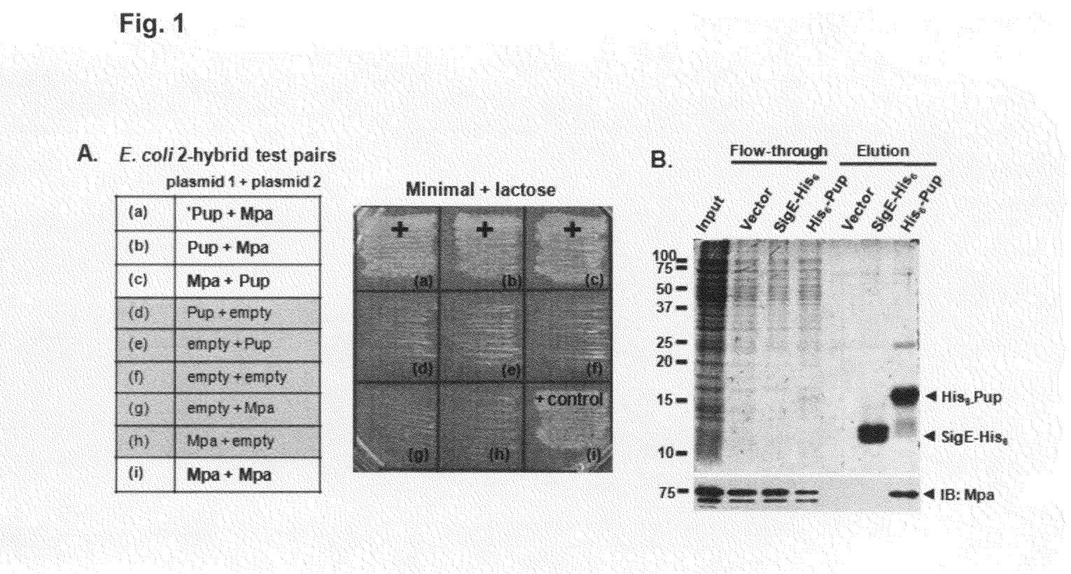

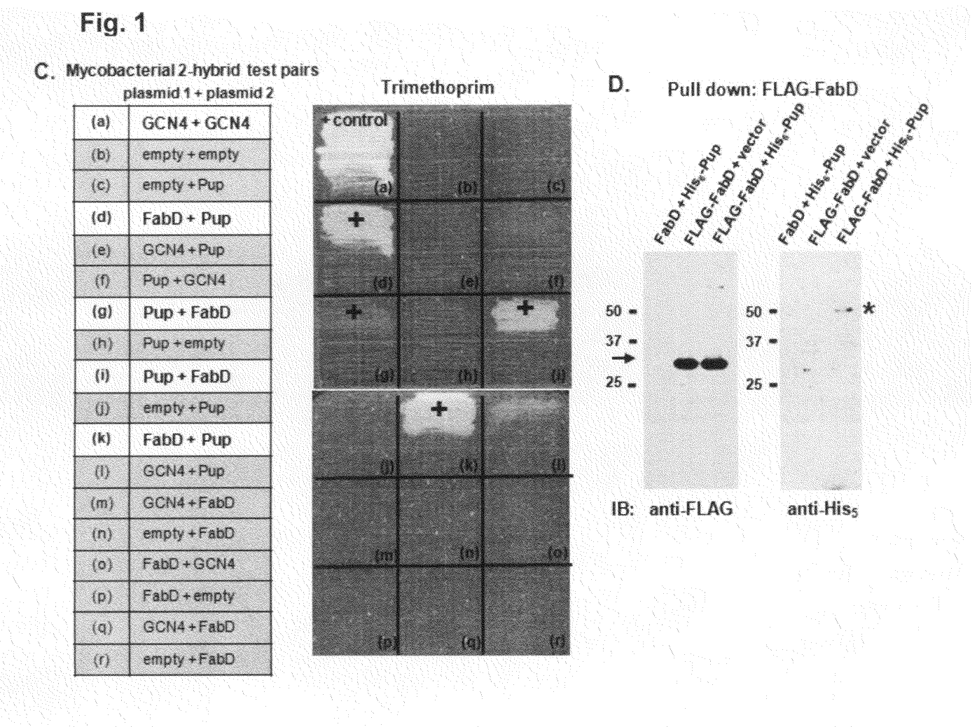

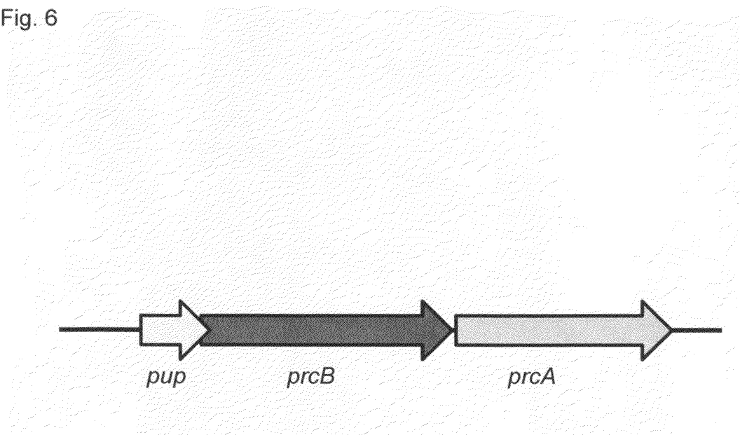

As indicated above, ubiquitin-like genes have not been identified in the Mtb genome, suggesting that substrate targeting to the Mtb proteasome occurs via an ubiquitin-independent mechanism. To further define the Mtb proteasome system, the present inventors looked for proteins that interacted with Mpa using an E. coli bacterial two-hybrid (BTH) system (Karimova et al, Proc Natl Acad Sci USA 95, 5752 (1998); Pearce et al, Science 322, 1104 (2008; Epub Oct. 2, 2008). A fusion protein that encoded the last 26 amino acids of Rv2111c (henceforth referred to as Pup) interacted with the Mpa bait fusion [FIG. 1A(a) (Pearce et al, Science 322, 1104 (2008; Epub Oct. 2, 2008)]. Full length Pup also specifically interacted with Mpa [FIGS. 1A(b,c)]. The pup gene has been acknowledged in the literature (Knipfer et al, Mol Microbiol 25, 375 (1997); Tamura et al., Curr Biol 5, 766 (Jul. 1, 1995)) but the function of Pup was unknown. pup homologues have so far only been identified in Actinobacteria. In Mtb, pup is part of a putative operon with the proteasome core genes prcB and prcA (FIG. 6). The stop codon of pup overlaps with the start codon of prcB, suggesting that these genes are co-transcribed and translationally coupled. pup is predicted to encode a 64 amino acid protein with a molecular weight of 6.9 kD. However, recombinant Pup purified from E. coli migrates to a position around 15 kD in a denaturing polyacrylamide gel (FIG. 1B).

The present inventors then tested the Pup/Mpa interaction in vitro using nickel-nitrilotriacetic acid agarose (NiNTA) bound with purified His₆-Pup, and Pup was able to bind Mpa [(FIG. 1B) (Pearce et al, Science 322, 1104 (2008; Epub Oct. 2, 2008)]. Mpa was not retained by agarose that had been pre-incubated with E. coli lysate or with SigE-His₆, a Salmonella typhimurium protein that is similar in size and charge to Pup (Darwin et al, J Bacteriol 183, 1452 (February, 2001)). Therefore, Pup can specifically and non-covalently interact with Mpa in an E. coli lysate, under native conditions.

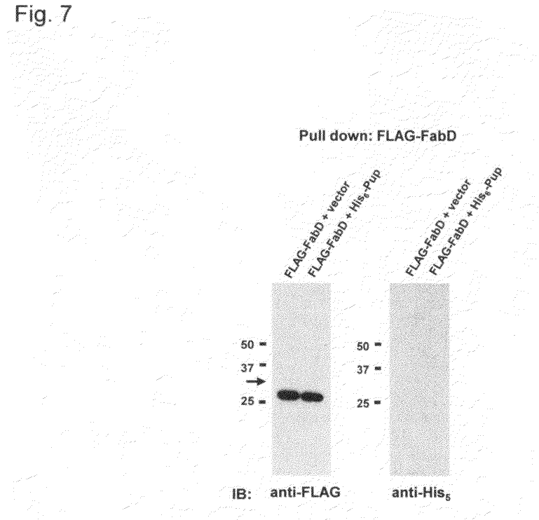

Genetic and biochemical experiments using E. coli to test for interactions between Pup and other Mtb proteasome components were unsuccessful. Based on these negative data, the present inventors hypothesized that E. coli was missing cofactors that were necessary to promote certain Mtb protein-protein interactions. A mycobacterial protein fragment complementation (M-PFC) assay that tests for interactions in Mycobacterium smegmatis (Msm) was chosen to address this potentiality (Singh et al, Proc Natl Acad Sci USA 103, 11346 (Jul. 25, 2006)). The present inventors tested various pairings of proteasome components and substrates and, surprisingly, observed a strong positive interaction between Pup and the proteasome substrate FabD [FIG. 1C (a, d, g, k)]. To further test the Pup interaction with FabD, constructs encoding FLAG-FabD and His₆-Pup in Msm were expressed. Antibodies to FLAG detected FLAG-FabD at the predicted size of ˜30 kD when purified from Msm (FIG. 1D, left panel). Unexpectedly, when the present inventors used antibodies to detect His₆-Pup, a ˜45 kD species was observed when FLAG-FabD and His₆-Pup were co-produced in mycobacteria (FIG. 1D, right panel). This ˜45 kD species likely represented a complex between FabD and Pup. The complex was highly stable as it was maintained under reducing and denaturing conditions, unlike the Mpa/Pup interaction observed in E. coli (FIG. 1B). When FLAG-FabD was purified from an E. coli strain making His₆-Pup, the ˜45 kD species could not be detected (FIG. 7). Taken together, these results show that Pup interacts with an Mtb proteasome substrate in a manner that is not supported in E. coli, and requires Mycobacterium-specific factors.

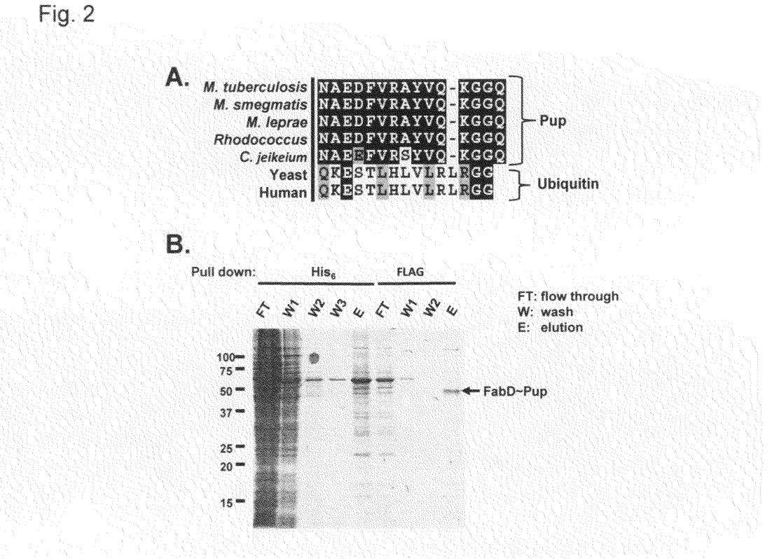

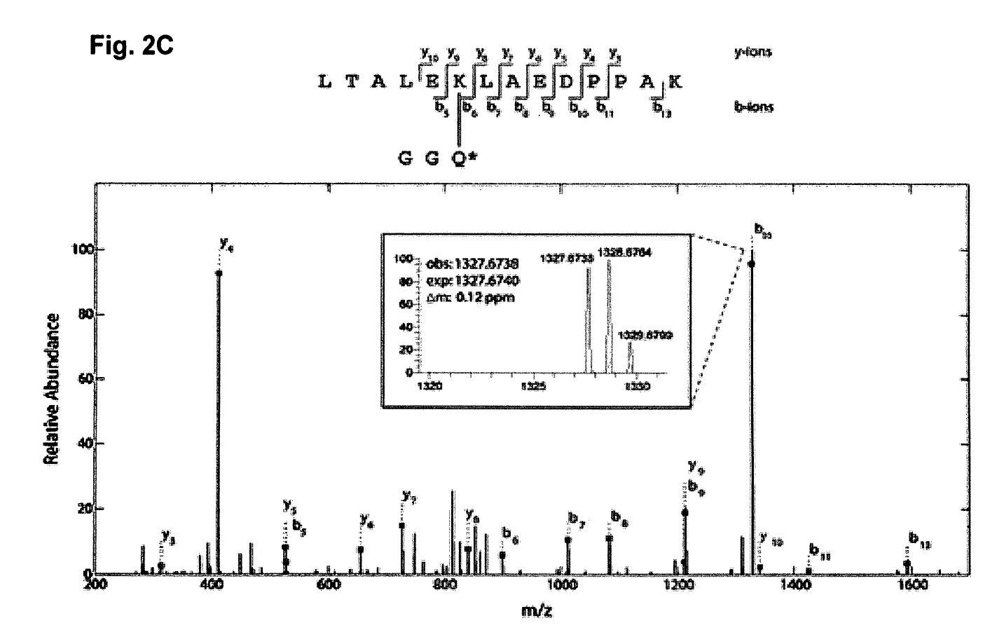

The formation of a stable complex between our model substrate FabD and Pup was reminiscent of the covalent attachment of ubiquitin to proteasome substrates in eukaryotes. Sequence and structural prediction comparisons between Pup and ubiquitin showed no overall homology. Conservation of either of the basic amino acids arginine (Arg) or Lys, followed by two glycines (Gly) at the C-termini (FIG. 2A) was, however, observed. This di-Gly motif is conserved in most members of the ubiquitin-like protein (Ubl) family, and is usually followed by one or more amino acids, [reviewed in Hochstrasser, Nat Cell Biol 2, E153 (August, 2000)]. The C-termini of Ubls are generally processed to expose the di-Gly, and then activated for conjugation to substrate proteins through a series of enzyme-catalyzed reactions [reviewed in Kerscher et al, Annu Rev Cell Dev Biol 22, 159 (2006)]. The terminal Gly of ubiquitin is essential for the formation of an isopeptide bond with the Lys of a substrate [reviewed in Hershko et al, Annu Rev Biochem 67, 425 (1998)].

The presence of a di-Gly motif in Pup prompted the present inventors to characterize the Pup˜FabD interaction using mass spectrometry (MS) (Kirkpatrick et al, Nat Cell Biol 7, 750 (August, 2005)). The stability of the FLAG-FabD˜His₆-Pup facilitated use of a tandem affinity purification to recover the ˜45 kD protein to high purity (FIG. 2B). The ˜45 kD band was excised from a denaturing polyacrylamide gel, in-gel digested with trypsin and analyzed by nano-LC/MS. MS analysis confirmed the presence of both FLAG-FabD and His₆-Pup. MS analysis of ubiquitylated substrates typically identifies substrate peptides with the tryptic Gly-Gly fragment covalently attached to a lysine (21). Given the Pup C-terminal sequence [Gly-Gly-glutamine (Gln)], a tandem MS/MS search was performed allowing for either a Gly-Gly or Gly-Gly-Gln modification. The latter revealed several spectral matches to a FabD tryptic peptide with the Pup C-terminal sequence attached through an isopeptide bond to Lys 173 of FabD, as is seen with ubiquitin and related modifiers [reviewed in Schwartz et al, Trends Biochem Sci 28, 321 (June, 2003)]. The precursor mass deviation, however, suggested a de-amidation event (ΔM=+0.984), pointing to a likely C-terminal Gln→Glu conversion. This hypothesis was confirmed with high-resolution tandem MS/MS (FIG. 2C). This result proves that the Gln following the di-Gly of Pup was not removed, which stands in marked contrast to expectations based on canonical Ubl protein activation. Instead, the Gln was either converted into Glu, possibly via a de-amidation reaction, or it was exchanged for Glu. In light of these results, the Glu appears to be biologically significant as Pup homologues in a number of Actinobacteria naturally terminate in Glu (FIG. 8), and a Gln-containing form of this peptide was never detected. Taken together, these data establish that Pup is covalently bound to a specific Lys residue of an Mtb proteasome substrate in a manner analogous to the conjugation of ubiquitin to eukaryotic proteasome substrates. Additionally, findings presented herein allude to a novel mechanism for Pup conjugation where the C-terminus is not removed, indicating that the Gly is not directly conjugated to Lys in FabD.

The present inventors previously showed that FabD and other Mtb proteasome substrates accumulate in mpa and pafA mutants (Pearce et al., EMBO J. 25, 5423 (2006)). If Pup, like ubiquitin, targets proteins for degradation, it was postulated that pupylated FabD would also accumulate in these mutants. An immunoblot revealed the accumulation of epitope-tagged FabD when affinity purified from the mpa and pafA strains compared to WT Mtb (FIG. 3A, left). Upon closer inspection, a ˜45 kD band was observed in WT bacteria (FIG. 3A, left, asterisk). To investigate the identity of this band, Pup antibodies were used to detect pupylated FabD in the same samples. The ˜45 kD protein was observed in WT Mtb, and an accumulation of this species was also noted in the mpa mutant (FIG. 3A, right). Unexpectedly, there was no pupylated FabD detected in the pafA strain (FIG. 3A, right), despite the accumulation of unpupylated FabD. Taken together this result suggests that PafA is involved in the pupylation of Mtb proteasome substrates, and in the absence of proteasome activity, pupylated protein accumulates. When the present inventors purified epitope-tagged dihydrolipoamide acyltransferase (DlaT), which is not an Mtb proteasome substrate (Pearce et al., EMBO J. 25, 5423 (2006)), a pupylated species was not detected, demonstrating that pupylation is specific for Mtb proteasome substrates (FIG. 3B).

The accumulation of pupylated FabD in the mpa strain supports the notion that Pup is involved in targeting substrates to the Mtb proteasome. Furthermore, FIG. 3A illustrates that the pupylated FabD exists at extremely low steady state levels. This might indicate that the transition from an unpupylated to a pupylated state is a tightly regulated process, much like that of Ubl conjugation (Kerscher et al, Annu Rev Cell Dev Biol 22, 159 (2006); Geiss-Friedlander et al, Nat Rev Mol Cell Biol 8, 947 (December, 2007)).

If Pup acts like ubiquitin, then multiple pupylated proteins could exist in Mtb. Immunoblot analysis using the anti-Pup antibody against soluble proteins from WT and mpa Mtb strains revealed a ladder of proteins (FIG. 3C). A number of pupylated proteins appeared more abundant in the mpa lysate compared to the WT sample (FIG. 3C, arrows). Once again, no anti-Pup reactive bands were observed in the pafA sample (FIG. 3C), implying that this phenomenon extends to all targets of pupylation within the limits of detection. The unconjugated form of Pup was not detectable, suggesting that the bulk of Pup is conjugated to proteins at steady state, or is unstable in an unconjugated form. Because pafA is in an operon with pafBC, the present inventors also tested mutants of those genes for substrate pupylation (FIG. 3C). The level of pupylation did not differ between the WT strain and the pafB and pafC mutants, which agrees with previous work demonstrating that PafB and PafC do not seem to be involved in substrate degradation (Festa et al, J Bacteriol 189, 3044 (April, 2007)).

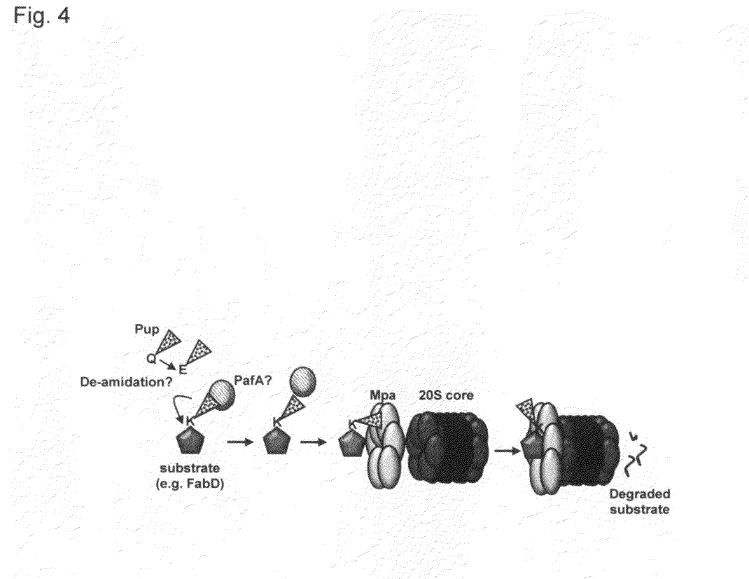

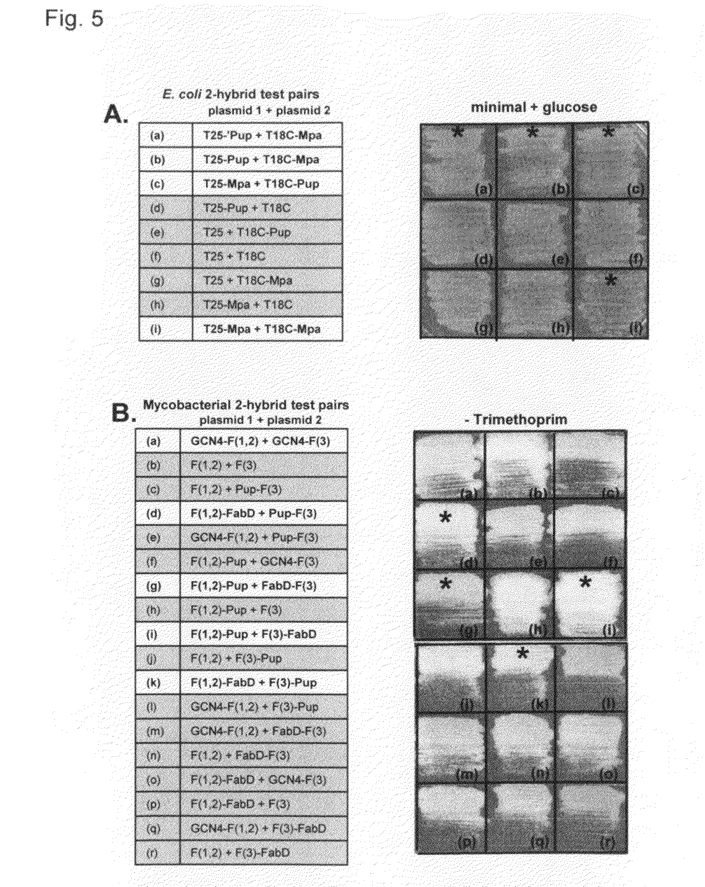

Pup, therefore, appears to be serving an ubiquitin-like function in a prokaryotic proteasome pathway (FIG. 4). The present inventors have demonstrated herein that Pup covalently conjugates to a specific Lys of an Mtb proteasome substrate. The present inventors have also shown that a lack of pupylated proteins in the Mtb pafA mutant correlates with substrate accumulation, suggesting that pupylation plays a role in substrate degradation. The proposed proteasomal ATPase Mpa is, therefore, likely to mediate the recognition of pupylated substrates into the proteasome via the non-covalent interaction with Pup detected in E. coli (FIG. 1B).

Although there are similarities between the ubiquitin and Pup systems, there are also striking differences. Unique aspects of pupylation may include the mechanism of Pup activation and conjugation to substrates, the chemistry involved in the linkage of Pup to Lys, the lack of polypupylated chains attached to substrates, and the involvement of PafA, a protein that shows no homology to any functionally defined protein. These findings suggest that PafA may play a part in conjugating Pup to substrates.

In summary, Pup is the first post-translational protein modifier to be identified in bacteria. Aside from degradation, the presence of small protein modifiers has important implications for other cellular processes in prokaryotes, especially when one considers the multitude of activities coordinated by ubiquitylation or SUMOylation in eukaryotes (Kerscher et al, Annu Rev Cell Dev Biol 22, 159 (2006); Geiss-Friedlander et al, Nat Rev Mol Cell Biol 8, 947 (December, 2007)). In prokaryotes, these activities could vary from subcellular protein sorting to the secretion of proteins out of the cell. The elucidation of novel ubiquitin-like pathways, like pupylation, provides new insight into biological processes in bacteria and may lead to the discovery of unique drug targets in deadly pathogens.

### Example II

In order to investigate and identify additional components that participate in the pupylation pathway and/or are acted upon by the pupylation pathway, a Pup affinity purification protocol was developed by the present inventors to identify polypeptides that interact with Pup. The Pup affinity reagent used in the purification protocol had 2 tags (His5-Strep-Pup) and was purified from Mtb under denaturing conditions. Tandem affinity purification (TAP) with His₆ and Strep II tags was used to purify pupylated proteins from wildtype Mtb. Similar TAP techniques have been used to identify the target of ubiquitylation and SUMOylation in eukaryotic cells (see Xu et al. Biochimica et biophysica acta (2006) 1764, 1940, the entire contents of which is incorporated herein in its entirety). His6-Strep-Pup (TAP-Pup) was first purified via the His6-tag under denaturing conditions to minimize secondary and non-specific interactions. To this end, soluble Mtb lysates from WT Mtb were incubated first with Ni-NTA agarose for enrichment of either His6-Pup or His6-Strep-Pup. His6-enriched proteins were then incubated with Strep-Tactin Superflow beads for enrichment of His6-Strep-Pup. This Strep-tag purification step was performed under native conditions. A mock TAP procedure using only a His6-tagged Pup was performed as a control. Purified proteins were separated on a denaturing polyacrylamide gel (10% SDS-PAGE) and the gel was sliced into pieces for in-gel digestion and MS analysis.

As indicated above, peptide fragments of proteins purified using this protocol were analyzed by mass spectroscopy (MS). Table 2 presents a list of all the peptides identified by the aforementioned Pup affinity purification protocol developed by the present inventors. Of note, this list includes PafA and PafD, and thus validates the use of Pup to find novel enzymes in the pupylation pathway that could be targeted for drug development.

Proteins previously reported to be required for growth in mice and/or macrophages (e.g. Icl, PknG) were also identified among the proteins pulled down by Pup.

In an independent study, a BLASTP analysis was performed in order to search for PafA domains involved in pupylation. The analysis showed that PafA is highly similar (38% identity) to the predicted amino acid sequence of the protein encoded by the Rv2112c gene (named pafD). The analysis also revealed that PafA and PafD share the same homology domains (DUF275 and DUF245), indicating that these proteins may have similar functions. In addition, pafD is located upstream of pup suggesting a role in pupylation. A localization analysis using PSORT predicted that PafD has no N-terminus signal sequence, indicating that it is likely to be localized in the cytoplasm.

Additional structure analysis using HHpred prediction software (Soding et al. Nucleic Acids Res. 33 (Web Server issue), W244 (2005)) revealed that PafA is structurally similar to glutamine synthases from several organisms.

In order to elucidate the function of PafD, an anti-Pup immunoblot of M. tuberculosis whole cell lysates from WT and pafD mutant strains was performed. Samples containing equivalent cell numbers were resolved by 10% SDS-PAGE. Proteins were transferred to nitrocellulose membranes and probed individually with anti-PafD and anti-Pup polyclonal antibodies. Reactive bands were visualized with SuperSignal West Pico Chemiluminescent Substrate (Thermo Scientific). The results show that there is a significant reduction of pupylated proteins in the pafD mutant strain compared to WT. Moreover, pupylation is recovered in the complemented pafD mutant strain (either with the WT or PafD-His₆ version). This result demonstrates that PafD has an important role in the pupylation pathway.

Table 3 presents a list of peptides to which Pup is thought to be attached based on changes in the mass of the tryptic fragment. In that the present inventors have demonstrated that Pup is covalently attached to these peptides, they are strong candidates for bona fide pupylation targets.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An isolated polypeptide comprising SEQ ID NO: 2, wherein said polypeptide is conjugated to proteasome substrates and conjugation thereof is associated with substrate degradation.

2. An isolated nucleic acid sequence encoding the isolated polypeptide of claim 1.

3. A vector comprising the isolated nucleic acid sequence of claim 2, wherein said isolated nucleic acid sequence is operably linked to regulatory elements.

4. The vector of claim 3, wherein said vector is an expression vector.

5. The isolated nucleic acid sequence of claim 2, wherein said isolated nucleic acid is SEQ ID NO: 1.

6. An antibody immunologically specific for the isolated polypeptide of claim 1.

7. The antibody of claim 6, wherein the antibody is a polyclonal antibody.

8. The antibody of claim 6, wherein the antibody is a monoclonal antibody.

9. A method for identifying a proteasome substrate in a bacterial cell, the method comprising:
a) isolating polypeptides covalently conjugated to Pup from a bacterial cell; and
b) characterizing the polypeptides covalently conjugated to Pup to identify each of the polypeptides conjugated to Pup, wherein identifying each of the polypeptides conjugated to Pup identifies a proteasome substrate in the bacterial cell.

10. The method of claim 9, wherein the bacterial cell is a Mycobacterium.

11. The method of claim 9, wherein polypeptides covalently conjugated to Pup are isolated by affinity purification of the covalently conjugated Pup.

12. The method of claim 11, wherein polypeptides covalently conjugated to Pup are isolated by affinity purification using antibodies immunologically specific for Pup.

13. The method of claim 9, wherein the bacterial cell expresses a tagged form of Pup comprising Pup and a tag and polypeptides covalently conjugated to the tagged form of Pup are isolated by affinity purification of the tag.

14. The method of claim 9, wherein polypeptides covalently conjugated to Pup are characterized by sequencing at least part of each of the polypeptides conjugated to Pup.

15. A method for identifying modulators of Pup activity in a bacterial cell, the method comprising:
(a) providing at least two bacterial cells, wherein each bacterial cell expresses Pup;
(b) incubating at least one bacterial cell expressing Pup in the presence of an agent and at least one cell expressing Pup in the absence of the agent;
c) isolating and characterizing polypeptides covalently conjugated to Pup from the at least one bacterial cell expressing Pup in the presence of the agent and the at least one cell expressing Pup in the absence of the agent; and
d) comparing the polypeptides covalently conjugated to Pup from the at least one bacterial cell expressing Pup in the presence of the agent to the polypeptides covalently conjugated to Pup from the at least one cell expressing Pup in the absence of the agent, wherein a change in the population of polypeptides covalently conjugated to Pup in the presence and absence of the agent identifies an agent that is a modulator of Pup activity in the bacterial cell.

16. The method of claim 15, wherein the change in the population of polypeptides covalently conjugated to Pup is a change in an amount of a particular polypeptide covalently conjugated to Pup or a change in types of polypeptides conjugated to Pup.

17. The method of claim 15, wherein the bacterial cell is a Mycobacterium.

18. The method of claim 15, wherein the at least two bacterial cells express a tagged form of Pup comprising Pup and a tag and polypeptides covalently conjugated to the tagged form of Pup are isolated by affinity purification of the tag.

19. The method of claim 15, wherein polypeptides covalently conjugated to Pup are characterized by sequencing at least part of each of the polypeptides conjugated to Pup.

20. A method for identifying an enzyme that covalently attaches Pup to a proteasome substrate in a bacterial cell, the method comprising:
(a) providing a population of bacterial cells;
(b) introducing an expression library of nucleic acid sequences isolated from a Pup expressing bacterial cell into the population of bacterial cells to generate a population of bacterial cells expressing exogenous nucleic acids;
(c) characterizing polypeptides covalently conjugated to Pup in the population of bacterial cells expressing exogenous nucleic acids to identify an exogenous nucleic acid that increases covalent conjugation of Pup polypeptides when expressed.

21. The method of claim 20, wherein the population of bacterial cells comprises proteasomes.

22. The method of claim 20, wherein each bacterial cell of the population of bacterial cells expresses Pup.

23. The method of claim 20, wherein the bacterial cells are Mycobacterium.

24. The method of claim 20, wherein each bacterial cell of the population of bacterial cells expresses a tagged form of Pup comprising Pup and a tag.

25. A kit for expressing and purifying a fusion protein in a eukaryotic cell, the method comprising:
(a) expressing the fusion protein in the eukaryotic cell, wherein the fusion protein comprises a polypeptide sequence fused in frame to a Pup sequence; and
(b) purifying the fusion protein by affinity purification.

26. The kit of claim 25, wherein the fusion protein further comprises an epitope tag and is purified by affinity purification of the epitope tag.

27. The kit of claim 26, wherein the epitope tag is a histidine tag

28. The kit of claim 25, wherein the fusion protein is purified by affinity purification of the Pup sequence.

29. The kit of claim 25, wherein the polypeptide sequence is fused to the Pup sequence via a linker.

30. The kit of claim 29, wherein the linker comprises a cleavage site for a protease.
